(19) United States
(12) Patent Application Publication
(10) Pub. No.: US 2024/0067742 A1
(43) Pub. Date: Feb. 29, 2024

(54) ENHANCED ANTI-HVEM ANTIBODIES AND USE THEREOF

(71) Applicants: 4C BIOMED LIMITED, London (GB); SHEBA IMPACT LTD., Ramat Gan (IL)

(72) Inventors: Eyal GREENBERG, Even Yehuda (IL); Gilli GALORE-HASKEL, Tel Mond (IL); Efrat MERHAVI-SHOHAM, Rehovot (IL); Gal MARKEL, Ramat Gan (IL)

(21) Appl. No.: 18/375,657

(22) Filed: Oct. 2, 2023

Related U.S. Application Data
(63) Continuation of application No. PCT/IL2022/050348, filed on Mar. 31, 2022.
(60) Provisional application No. 63/169,335, filed on Apr. 1, 2021.

(51) Int. Cl. C07K 16/28; A61P 35/00; G01N 33/68

(52) U.S. Cl. CPC C07K 16/2878; A61P 35/00; G01N 33/6863; C07K 2317/565; G01N 2800/52

(57) ABSTRACT

Antibodies or antigen binding fragments thereof that bind HVEM, inhibit HVEM-BTLA interaction and allow binding to LIGHT are provided. Methods of treating disease with these antibodies or antigen binding fragments thereof, nucleic acid molecules encoding these antibodies or antigen binding fragments thereof and kits comprising these antibodies or antigen binding fragments thereof are also provided.

### CROSS REFERENCE TO RELATED APPLICATIONS

This application is a Bypass Continuation of PCT Patent Application No. PCT/IL2022/050348 having International filing date of Mar. 31, 2022, which claims the benefit of priority of U.S. Provisional Patent Application No. 63/169,335, filed Apr. 1, 2021, all of which is incorporated herein by reference in their entirety.

### FIELD OF INVENTION

The contents of the electronic sequence listing (4CB-P-007-US.xml; Size: 199,580 bytes; and Date of Creation: Oct. 29, 2023) is herein incorporated by reference in its entirety.

### FIELD OF INVENTION

The present invention is in the field of immunotherapy.

### BACKGROUND OF THE INVENTION

Immunotherapies designed to enhance an immune response are considered activating immunotherapies and are at the forefront of cancer treatment. Currently, immune checkpoint blockade therapy is successfully being used for treatment of advanced non-small cell lung cancers (NSCLC), metastatic melanoma, advanced renal cell carcinoma (RCC) metastatic urothelial carcinoma, head and neck squamous cell carcinoma (HNSCC), MSI-high tumors, Merkel cell carcinoma and many others. A few such drugs, developed and manufactured by different companies, have shown great promise in different cancer types and have been FDA approved, including antibodies to the immune checkpoints programmed cell death protein 1 receptor (PD1), programmed cell death protein 1 ligand (PDL1) and cytotoxic T lymphocyte-associated protein 4 (CTLA-4). Patient response rate, however, is still not optimal, as only 10%-40% of treated patients usually benefit, and at the same time patients may suffer from Post Immunotherapy Treatments Side-Effects. In addition, treatment with these antibodies or antigen binding fragments thereof, may induce resistance through upregulation of additional immune checkpoints. Combination of anti-PD1 and anti-CTLA-4 therapy in melanoma patients has demonstrated higher response rate (60%) as compared to single antibody or antigen binding fragment thereof, however this combination therapy involves also severe treatment-related adverse effects. Thus, there is a clear need for new antitumor immune activating antibodies or antigen binding fragments thereof.

Herpesvirus entry mediator (HVEM) is a protein found on the surface of various cell types, including hematopoietic and non-hematopoietic cells. HVEM acts as a receptor for canonical TNF-related ligands such as LIGHT and LTα, thus acting as a signaling receptor. However, it also acts as a ligand for immunoglobulin (Ig) superfamily molecules such as inhibitory receptors BTLA and CD160. Therefore, bidirectional signaling is possible for the HVEM-mediated signaling network, which can be involved in positive or negative immunological reactions under different contexts. Dysregulation of this network is involved in the pathogenesis of autoimmune diseases, inflammatory diseases as well as cancer, making HVEM a target for immunotherapy. Antibodies or antigen binding fragments thereof that can block HVEM inhibitory signaling, particularly through BTLA interaction, while preserving activating signaling are greatly needed.

### SUMMARY OF THE INVENTION

The present invention provides antibodies or antigen binding fragments thereof that bind HVEM, inhibit HVEM-BTLA interaction, and inhibit downstream BTLA signaling. Methods of treating disease with these antibodies or antigen binding fragments thereof, nucleic acid molecules encoding these antibodies or antigen binding fragments thereof and kits comprising these antibodies or antigen binding fragments thereof are also provided.

According to a first aspect, there is provided an antibody or antigen binding fragment thereof, comprises three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 49 (X₁IX₂X₃X₄X₅X₁₈X₇X₁₉YYADSVX₂₀G) wherein X₁ is A, G or N, X₂ is S, N, G or Y, X₃ is G or S, X₄ is S, N or P, X₅ is G or P, X₁₈ is any amino acid other than C, X₇ is S, Y, G or R, X₁₉ is any amino acid other than S or C, and X₂₀ is any amino acid, and CDR-H3 comprises the amino acid sequence as set forth in SEQ ID NO: 18 (AX₉X₁₀X₁₁X₁₂X₁₃X₁₄YX₁₅DY) wherein X₉ is P or S, X₁₀ is G or Y, X₁₁ is D or R, X₁₂ is Y, N, P or S, X₁₃ is T or Y, X₁₄ is A or N and X₁₅ is F, G or Y, CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 19 (QQYGSX₁₆PPX₁₇T) wherein X₁₆ is S or Y and X₁₇ is Y or L; and wherein the antibody or antigen binding fragment thereof does not comprise all of a CDR-H2 comprising the amino acid sequence as set forth in SEQ ID NO: 2 (AISGSGGSTYYADSVKG), a CDR-H3 comprising the amino acid sequence as set forth in SEQ ID NO: 3 (APGDYTAYFDY) and a CDR-L3 comprising the amino acid sequence as set forth in SEQ ID NO: 6 (QQYGSSPPYT).

According to another aspect, there is provided a pharmaceutical composition comprising an antibody or antigen binding fragment of the invention and a pharmaceutically acceptable carrier, excipient or adjuvant.

According to another aspect, there is provided a method of treating a disease or condition characterized by HVEM positive cells in a subject in need thereof, the method comprising administering to the subject the pharmaceutical composition of the invention or an antibody or antigen binding fragment of the invention, thereby treating the disease or condition.

According to another aspect, there is provided a method of determining suitability of a subject to be treated by a method of the invention, comprising obtaining a disease sample from the subject and determining HVEM levels in the sample, wherein positive expression of HVEM indicates the subject is suitable for a method of treatment of the invention.

According to another aspect, there is provided a method of detecting HVEM in a sample, the method comprising contacting the sample with an antibody or antigen binding fragment of the invention, thereby detecting HVEM.

According to another aspect, there is provided a nucleic acid molecule encoding an antibody or antigen binding fragment of the invention.

According to another aspect, there is provided a kit comprising, a pharmaceutical composition of the invention and at least one of:
a. an anti-PD-1/PD-L1 based immunotherapy;
b. a label stating the pharmaceutical composition of the invention is for use with an anti-PD-1/PD-L1 based immunotherapy; and
c. a secondary detection molecule for detecting at least one antibody or antigen binding fragment thereof of the invention.

According to some embodiments, CDR-H2 comprises the amino acid sequence as set forth in SEQ ID NO: 17 (X₁IX₂X₃X₄X₅X₆X₇X₂1YYADSVX₈G) wherein X₁ is A, G or N, X₂ is S, N, G or Y, X₃ is G or S, X₄ is S, N or P, X₅ is G or P, X₆ is G, D, S, E, Q or N, X₇ is S, Y, G or R, X₂₁ is T, A, E, G or N and X₈ is E or K.

According to some embodiments, X₂₀ is any amino acid other than C or S.

According to some embodiments, X₂₀ is any non-positively charged amino acid.

According to some embodiments, X₂₀ is K or E.

According to some embodiments, CDR-H2 comprises an amino acid sequence selected from: SEQ ID NO: 2, SEQ ID NO: 20 (GINGNGDYTYYADSVKG), SEQ ID NO: 21 (AIGGSGSGTYYADSVKG), SEQ ID NO: 22 (NIYSNPNRTYYADSVEG), SEQ ID NO: 23 (NINGPGNGTYYADSVEG), SEQ ID NO: 47 (NIYSNPNRTYYADSVKG), SEQ ID NO: 48: (AISGSGGSTYYADSVEG), SEQ ID NO: 57 (NIYSNPDRTYYADSVEG), SEQ ID NO: 58 (NIYSNPERTYYADSVEG), SEQ ID NO: 59 (NIYSNPGRTYYADSVEG), SEQ ID NO: 60 (NIYSNPQRTYYADSVEG), SEQ ID NO: 61 (NIYSNPSRTYYADSVEG), SEQ ID NO: 62 (NIYSNPNRAYYADSVEG), SEQ ID NO: 63 (NIYSNPNREYYADSVEG), SEQ ID NO: 64 (NIYSNPNRGYYADSVEG) and SEQ ID NO: 65 (NIYSNPNRNYYADSVEG).

According to some embodiments, CDR-H3 comprises an amino acid sequence selected from: SEQ ID NO: 3, SEQ ID NO: 24 (ASYRNYNYGDY), SEQ ID NO: 25 (ASYDPTNYYDY) and SEQ ID NO: 26 (ASYRSTNYFDY).

According to some embodiments, CDR-L3 comprises an amino acid sequence selected from: SEQ ID NO: 6 and SEQ ID NO: 27 (QQYGSYPPLT).

According to some embodiments, CDR-H1 comprises the amino acid sequence set forth in SEQ ID NO: 1, CDR-H2 comprises an amino acid sequence selected from SEQ ID NO: 20, SEQ ID NO: 21, SEQ ID NO: 22, SEQ ID NO: 23, SEQ ID NO: 47, SEQ ID NO: 48, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64 and SEQ ID NO: 65, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6.

According to some embodiments, CDR-H1 comprises the amino acid sequence set forth in SEQ ID NO: 1, CDR-H2 comprises the amino acid sequence set forth in SEQ ID NO: 2, CDR-H3 comprises an amino acid sequence selected from SEQ ID NO: 24, SEQ ID NO: 25 and SEQ ID NO: 26, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6.

According to some embodiments, CDR-H1 comprises the amino acid sequence set forth in SEQ ID NO: 1, CDR-H2 comprises the amino acid sequence set forth in SEQ ID NO: 2, CDR-H3 comprises the amino acid sequence set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 27.

According to some embodiments, the antibody or antigen binding fragment of the invention comprises a heavy chain comprising a sequence selected from

(SEQ ID NO: 7)
QVQLVQSGGGLVQPGGSLRLSCAASGFTFSSYAMSWVRQAPGKGLEWVSA
ISGSGGSTYYADSVKGRFTISRDNSKNTLYLQMNSLRAEDTAVYYCAKAP
GDYTAYFDYWGQGTLVTVSS,
(SEQ ID NO: 28)
QVQLVQSGGGLVQPGGSLRLSCAASGFTFSSYAMSWVRQAPGKGLEWVSG
INGNGDYTYYADSVKGRFTISRDNSKNTLYLQMNSLRAEDTAVYYCAKAP
GDYTAYFDYWGQGTLVTVSS,
(SEQ ID NO: 29)
QVQLVQSGGGLVQPGGSLRLSCAASGFTFSSYAMSWVRQAPGKGLEWVSA
IGGSGSGTYYADSVKGRFTISRDNSKNTLYLQMNSLRAEDTAVYYCAKAP
GDYTAYFDYWGQGTLVTVSS,
(SEQ ID NO: 30)
QVQLVQSGGGLVQPGGSLRLSCAASGFTFSSYAMSWVRQAPGKGLEWVSN
IYSNPNRTYYADSVEGRFTISRDNSKNTLYLQMNSLRAEDTAVYYCAKAP
GDYTAYFDYWGQGTLVTVSS,
(SEQ ID NO: 31)
QVQLVQSGGGLVQPGGSLRLSCAASGFTFSSYAMSWVRQAPGKGLEWVSN
INGPGNGTYYADSVEGRFTISRDNSKNTLYLQMNSLRAEDTAVYYCAKAP
GDYTAYFDYWGQGTLVTVSS,
(SEQ ID NO: 50)
QVQLVQSGGGLVQPGGSLRLSCAASGFTFSSYAMSWVRQAPGKGLEWVSN
IYSNPNRTYYADSVKGRFTISRDNSKNTLYLQMNSLRAEDTAVYYCAKAP
GDYTAYFDYWGQGTLVTVSS,
(SEQ ID NO: 51)
QVQLVQSGGGLVQPGGSLRLSCAASGFTFSSYAMSWVRQAPGKGLEWVSA
ISGSGGSTYYADSVEGRFTISRDNSKNTLYLQMNSLRAEDTAVYYCAKAP
GDYTAYFDYWGQGTLVTVSS,
(SEQ ID NO: 66)
QVQLVQSGGGLVQPGGSLRLSCAASGFTFSSYAMSWVRQAPGKGLEWVSN
IYSNPDRTYYADSVEGRFTISRDNSKNTLYLQMNSLRAEDTAVYYCAKAP
GDYTAYFDYWGQGTLVTVSS,
(SEQ ID NO: 67)
QVQLVQSGGGLVQPGGSLRLSCAASGFTFSSYAMSWVRQAPGKGLEWVSN
IYSNPERTYYADSVEGRFTISRDNSKNTLYLQMNSLRAEDTAVYYCAKAP
GDYTAYFDYWGQGTLVTVSS,
(SEQ ID NO: 68)
QVQLVQSGGGLVQPGGSLRLSCAASGFTFSSYAMSWVRQAPGKGLEWVSN
IYSNPGRTYYADSVEGRFTISRDNSKNTLYLQMNSLRAEDTAVYYCAKAP
GDYTAYFDYWGQGTLVTVSS,
(SEQ ID NO: 69)
QVQLVQSGGGLVQPGGSLRLSCAASGFTFSSYAMSWVRQAPGKGLEWVSN
IYSNPQRTYYADSVEGRFTISRDNSKNTLYLQMNSLRAEDTAVYYCAKAP
GDYTAYFDYWGQGTLVTVSS,
(SEQ ID NO: 70)
QVQLVQSGGGLVQPGGSLRLSCAASGFTFSSYAMSWVRQAPGKGLEWVSN
IYSNPSRTYYADSVEGRFTISRDNSKNTLYLQMNSLRAEDTAVYYCAKAP
GDYTAYFDYWGQGTLVTVSS,
(SEQ ID NO: 71)
QVQLVQSGGGLVQPGGSLRLSCAASGFTFSSYAMSWVRQAPGKGLEWVSN
IYSNPNRAYYADSVEGRFTISRDNSKNTLYLQMNSLRAEDTAVYYCAKAP
GDYTAYFDYWGQGTLVTVSS,
(SEQ ID NO: 72)
QVQLVQSGGGLVQPGGSLRLSCAASGFTFSSYAMSWVRQAPGKGLEWVSN
IYSNPNREYYADSVEGRFTISRDNSKNTLYLQMNSLRAEDTAVYYCAKAP
GDYTAYFDYWGQGTLVTVSS,
(SEQ ID NO: 73)
QVQLVQSGGGLVQPGGSLRLSCAASGFTFSSYAMSWVRQAPGKGLEWVSN
IYSNPNRGYYADSVEGRFTISRDNSKNTLYLQMNSLRAEDTAVYYCAKAP
GDYTAYFDYWGQGTLVTVSS,
(SEQ ID NO: 74)
QVQLVQSGGGLVQPGGSLRLSCAASGFTFSSYAMSWVRQAPGKGLEWVSN
IYSNPNRNYYADSVEGRFTISRDNSKNTLYLQMNSLRAEDTAVYYCAKAP
GDYTAYFDYWGQGTLVTVSS,
(SEQ ID NO: 32)
QVQLVQSGGGLVQPGGSLRLSCAASGFTFSSYAMSWVRQAPGKGLEWVSA
ISGSGGSTYYADSVKGRFTISRDNSKNTLYLQMNSLRAEDTAVYYCAKAS
YRNYNYGDYWGQGTLVTVSS,
(SEQ ID NO: 33)
QVQLVQSGGGLVQPGGSLRLSCAASGFTFSSYAMSWVRQAPGKGLEWVSA
ISGSGGSTYYADSVKGRFTISRDNSKNTLYLQMNSLRAEDTAVYYCAKAS
YDPTNYYDYWGQGTLVTVSS,
and
(SEQ ID NO: 34)
QVQLVQSGGGLVQPGGSLRLSCAASGFTFSSYAMSWVRQAPGKGLEWVSA
ISGSGGSTYYADSVKGRFTISRDNSKNTLYLQMNSLRAEDTAVYYCAKAS
YRSTNYFDYWGQGTLVTVSS.

According to some embodiments, the antibody or antigen binding fragment of the invention comprises a light chain comprising a sequence selected from:

(SEQ ID NO: 8)
ELVLTQSPATLSLSPGERATLSCRASQSVSSYLAWYQQKPGQAPRLLIYG
ASSRATGIPDRFSGSGSGTDFTLTISRLEPEDFAVYYCQQYGSSPPYTFG
QGTKVEIK
and
(SEQ ID NO: 35)
ELVLTQSPATLSLSPGERATLSCRASQSVSSYLAWYQQKPGQAPRLLIYG
ASSRATGIPDRFSGSGSGTDFTLTISRLEPEDFAVYYCQQYGSYPPLTFG
QGTKVEIK.

According to some embodiments, the antibody or antigen binding fragment of the invention comprises a heavy chain comprising an amino acid sequence selected from SEQ ID NO: 85-103.

According to some embodiments, the antibody or antigen binding fragment of the invention comprises a light chain comprising an amino acid sequence selected from SEQ ID NO: 104 and SEQ ID NO: 105.

According to some embodiments, the antibody or antigen binding fragment thereof of the invention, comprises a heavy chain comprising SEQ ID NO: 97 and a light chain comprising SEQ ID NO: 104.

According to some embodiments, the method of the invention further comprises inhibition or blockade of a non-HVEM immune checkpoint protein.

According to some embodiments, the method of the invention further comprises administering an anti-PD-1/PD-L1 based immunotherapy.

According to some embodiments, the method of the invention further comprises administering adoptive cell therapy.

According to some embodiments, the adoptive cell therapy comprises adoptive TIL therapy.

According to some embodiments, the adoptive cell therapy comprises administering a chimeric antigen receptor (CAR) expressing immune cell, wherein the CAR targets a non-HVEM protein on a surface of the HVEM expressing cells.

According to some embodiments, the disease or condition is an HVEM positive cancer or precancerous lesion.

According to some embodiments, the disease or condition is an infectious disease and wherein the infected cells comprise HVEM expression.

According to some embodiments, positive expression of HVEM comprises an elevated HVEM level as compared to a healthy sample or predetermined threshold.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Photograph of SDS-PAGE of various purified antibodies in non-reducing and reducing conditions. The heavy chain band of the H4 antibody runs high due to glycosylation, which is lost in the three variant antibodies in which the NXS/T glycosylation site is removed.

FIG. 2: Bar graph of binding to hHVEM coated plates by two antibodies of the invention (H4 and Par-K64E), as well as isotype control as the negative control and Parental as the positive control.

FIGS. 3A-3D: (3A-3B) Biacore (3A) multi-cycle kinetic and (3B) single-cycle kinetic raw sensorgrams and fitted curves with a 1:1 model for the binding to hHVEM of (3A) SEC purified antibodies H4, H4 T57A, H4 T57G, H4 T57N and parent antibody and (3B) supernatants of H4 N55G, H4 N55S, H4 N55D, H4 N55Q, H4N55E, H4T57E and parent antibody. (3C) A histogram overlay of binding of the 4 antibodies to hHVEM of the surface of CHO cells. (3D) Bar graph of hHVEM expression on CHO cells as detected by flow cytometry using the parental or H4 T57A antibodies at various concentrations.

FIGS. 4A-4H: Bar charts of absorbance read at 450 nm and 570 nm of ELISA on (4A-4E) recombinant hHVEM coated plates in the presence of (4A) parental, H4 T57A, H4 T57G and H4 T57N anti-HVEM antibodies, (4B) parental, H4 T57A, H4 T57G and H4 T57N anti-HVEM antibodies and recombinant hBTLA, (4C) parental, H4 T57A, H4 T57G and H4 T57N anti-HVEM antibodies and recombinant hLIGHT, (4D) parental, H4 and Par-K64E anti-HVEM antibodies and recombinant hBTLA, (4E) parental, H4 and Par-K64E anti-HVEM antibodies and recombinant hLIGHT, or (4F-4G) recombinant cHVEM coated plates in the presence of (4F) parental, H4 T57A, H4 T57G and H4 T57N anti-HVEM antibodies and (4G) parental, H4 T57A, H4 T57G and H4 T57N anti-HVEM antibodies and recombinant cBTLA. Detection of antibodies in 4A and 4F. Detection of BTLA in 4B, 4D and 4G. Detection of LIGHT in 4C and 4E. (4H) Line graph of absorbance at increasing antibody concentration in the presence of recombinant hBTLA.

FIGS. 5A-5B: Line graphs of specific cell killing of melanoma cells cocultured with TILs after a preincubation with (5A) the anti-HVEM antibodies or (5B) the anti-HVEM antibodies in combination with an anti-PD-1 antibody. **=p<0.01, ***=p<0.001.

FIGS. 6A-6B: Line graphs of CD107a positive TILs in coculture with (6A) a first HVEM-positive melanoma cell line, M-001 and (6B) a second HVEM-positive melanoma cell line, M-004. *=p<0.05, **=p<0.01, ***=p<0.001.

FIGS. 7A-7B: Line graphs of specific cell killing of primary ovary cancer cells cocultured with autologous PBMCs in the presence of (7A) the anti-HVEM antibodies or anti-PD1 antibody, or (7B) the anti-HVEM antibodies in combination with an anti-PD-1 antibody.

FIG. 8: Micrographs (Magnification×63, Zoom 1.5.) of FFPE CHO cells overexpressing hHVEM stained fluorescently with the parental antibody (left) or the H4 T57A antibody (right) and a secondary Cy3 antibody (red fluorescence). Nuclei are counterstained blue with DAPI.

### DETAILED DESCRIPTION OF THE INVENTION

The present invention, in some embodiments, provides antibodies or antigen binding fragments thereof that bind HVEM, inhibit HVEM-BTLA interaction, and do not significantly inhibit HVEM-LIGHT interaction. The present invention further concerns methods of treating HVEM positive disease in a subject in need thereof by administering these antibodies or antigen binding fragments thereof, nucleic acid molecules encoding these antibodies or antigen binding fragments thereof and kits comprising these antibodies or antigen binding fragments thereof.

It is well known that HVEM positive cancers can avoid immune surveillance by binding BTLA on the surface of immune cells. Engagement of BTLA produces an inhibitory signal within the immune cell, which reduces T cell activation and increases cancer survival. Inhibiting this signaling with antibodies that bind HVEM or antibodies that bind BTLA is known. International Patent Publication WO2020222235, herein incorporated by reference in its entirety, provides antibodies that specifically bind HVEM and do not inhibit the binding of other HVEM ligands such as LIGHT. HVEM is also expressed on the surface of immune cells where it plays both an inhibitory and activating role, depending on the context. Those antibodies not only block the HVEM-BTLA interaction by binding HVEM on pathogenic cells thus freeing immune cells from inhibition, but also allow HVEM to bind activating ligands (e.g., LIGHT). The instant invention is based on the finding of antibodies that are superior to those presented in WO2020222235. The antibodies of the invention bind to HVEM with greater strength (lower K_{D}) than do the already known anti-HVEM antibodies.

By a first aspect, there is provided an antibody or antigen binding fragment thereof, comprising three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 17 (X₁IX₂X₃X₄X₅X₆X₇X₂1YYADSVX₈G) wherein X₁ is A, G or N, X₂ is S, N, G or Y, X₃ is G or S, X₄ is S, N or P, X₅ is G or P, X₆ is G, D, S, E, Q or N, X₇ is S, Y, G or R, X₂₁ is T, A, E, G or N and X₈ is E or K, CDR-H3 comprises the amino acid sequence as set forth in SEQ ID NO: 18 (AX₉X₁₀X₁₁X₁₂X₁₃X₁₄YX₁₅DY) wherein X₉ is P or S, X₁₀ is G or Y, X₁₁ is D or R, X₁₂ is Y, N, P or S, X₁₃ is T or Y, X₁₄ is A or N and X₁₅ is F, G or Y, CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 19 (QQYGSX₁₆PPX₁₇T) wherein X₁₆ is S or Y and X₁₇ is Y or L.

By another aspect, there is provided an antibody or antigen binding fragment thereof, comprising three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 49 (X₁IX₂X₃X₄X₅X₁₈X₇X₁₉YYADSVX₂₀G) wherein X₁ is A, G or N, X₂ is S, N, G or Y, X₃ is G or S, X₄ is S, N or P, X₅ is G or P, X₁₈ is any amino acid other than C, X₇ is S, Y, G or R, X₁₉ is any amino acid other than S or C, and X₂₀ is any amino acid, and CDR-H3 comprises the amino acid sequence as set forth in SEQ ID NO: 18 (AX₉X₁₀X₁₁X₁₂X₁₃X₁₄YX₁₅DY) wherein X₉ is P or S, X₁₀ is G or Y, X₁₁ is D or R, X₁₂ is Y, N, P or S, X₁₃ is T or Y, X₁₄ is A or N and X₁₅ is F, G or Y, CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 19 (QQYGSX₁₆PPX₁₇T) wherein X₁₆ is S or Y and X₁₇ is Y or L.

In some embodiments, the antibody or antigen binding fragment thereof binds to HVEM. In some embodiments, the antibody or antigen binding fragment thereof has superior HVEM binding as compared to an antibody known in the art. In some embodiments, the antibody known in the art is the parental antibody. In some embodiments, the antibody known in the art is an antibody comprises three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 2 (AISGSGGSTYYADSVKG), CDR-H3 comprises the amino acid sequence as set forth in SEQ ID NO: 3 (APGDYTAYFDY), CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 6 (QQYGSSPPYT). It will be understood by a skilled artisan that there is more than one way to calculate CDRs. Heavy chain CDRS of SEQ ID NO: 1-3 and light chain CDRs of SEQ ID NO: 4-6 are according to the KABAT numbering system. In some embodiments, the antibody known in the art comprises the following CDRS: CDR-H1 comprises the amino acid sequence set forth in SEQ ID NO: 11 (GFTFSSYA), CDR-H2 comprises the amino acid sequence as set forth in SEQ ID NO: 12 (ISGSGGST), CDR-H3 comprises the amino acid sequence as set forth in SEQ ID NO: 13 (AKAPGDYTAYFDY), CDR-L1 comprises the amino acid sequence as set forth in SEQ ID NO: 14 (QSVSSY), CDR-L2 comprises the amino acid sequence as set forth in SEQ ID NO: 15 (GAS), and CDR-L3 comprises the amino acid sequence as set forth in SEQ ID NO: 16 (QQYGSSPPYT). Heavy chain CDRS of SEQ ID NO: 11-13 and light chain CDRs of SEQ ID NO: 14-16 are according to the IMGT numbering system. In some embodiments, the antibody known in the art is an antibody comprises a heavy chain variable region comprising or consisting of SEQ ID NO: 7 and a light chain variable region comprising or consisting of SEQ ID NO: 8. In some embodiments, the antibody known in the art is an antibody with a heavy chain of SEQ ID NO: 9 and a light chain of SEQ ID NO: 10.

In some embodiments, superior binding is stronger binding. In some embodiments, superior binding is binding with greater affinity. In some embodiments, superior binding is binding with a lower K_{D}. In some embodiments, the antibody is an anti-HVEM antibody. In some embodiments, the antibody or antigen binding fragment thereof is an HVEM blocking antibody. In some embodiments, the antibody or antigen binding fragment thereof blocks interaction between HVEM and BTLA. In some embodiments, the antibody or antigen binding fragment thereof, has superior blocking as compared to an antibody known in the art. In some embodiments, superior is stronger. In some embodiments, superior is longer lasting. In some embodiments, superior is more specific. In some embodiments, the antibody or antigen binding fragment thereof activates downstream signaling through HVEM. In some embodiments, the antibody or antigen binding fragment thereof, has superior activating as compared to an antibody known in the art. In some embodiments, the antibody or antigen binding fragment thereof inhibits downstream signaling through BTLA. In some embodiments, the antibody or antigen binding fragment thereof, has superior inhibition as compared to an antibody known in the art. In some embodiments, the antibody or antigen binding fragment thereof activates downstream signaling through the HVEM and inhibits downstream signaling through the BTLA. In some embodiments, inhibiting interaction between the HVEM and BTLA inhibits downstream signaling through the BTLA.

In some embodiments, HVEM is mammalian HVEM. In some embodiments, HVEM is rodent HVEM. In some embodiments, HVEM is monkey HVEM. In some embodiments, HVEM is human HVEM. In some embodiments, HVEM is any one of mouse, monkey and human HVEM. In some embodiments, HVEM is membrane bound HVEM. In some embodiments, HVEM is HVEM on a cell. In some embodiments, HVEM is HVEM on a cell surface. In some embodiments, HVEM is soluble HVEM.

In some embodiments, the cell is a pathogenic cell. In some embodiments, the cell is a cancerous cell. In some embodiments, the cell is a cell of a pathogen. In some embodiments, the cell is a bacterial cell. In some embodiments, the cell is a fungal cell. In some embodiments, the cell is a eukaryotic cell infected by a pathogen. In some embodiments, the cell is a cell infected by a bacterium. In some embodiments, the cell is a cell infected by a virus. In some embodiments, a pathogen is selected from a bacterium, a virus and a fungus.

In some embodiments, the cell is an immune cell. In some embodiments, the cell is a hematopoietic cell. In some embodiments, the immune cell is a T-cell. In some embodiments, the T-cell is a CD8 positive T-cell. In some embodiments, the T-cell is a cytotoxic CD8 positive T-cell. In some embodiments, the T-cell is a CD4 positive T-cell. In some embodiments, the T-cell is a CD4 positive helper T-cell. In some embodiments, the T-cell is selected from a CD8 positive and a CD4 positive T-cell. In some embodiments, the T-cell is a CD8 positive T-cell, a CD4 positive T-cell or both. In some embodiments, the immune cell is a gamma/delta T cell. In some embodiments, the immune cell is a tumor infiltrating lymphocyte (TIL). In some embodiments, the immune cell is not a peripheral blood immune cell. In some embodiments, the immune cell is a B-cell. In some embodiments, the immune cell is a natural killer (NK) cell. In some embodiments, the immune cell is a neutrophil. In some embodiments, the immune cell is a dendritic cell. In some embodiments, the immune cell is a macrophage. In some embodiments, the immune cell is a myeloid derived suppressor cell (MDSC). In some embodiments, the cell is selected from a T-cell, a B-cell, an NK cell, a neutrophil, a dendritic cell, an MDSC and a macrophage. In some embodiments, the cell is selected from a T-cell, a B-cell, an NK cell, a neutrophil, a dendritic cell, and a macrophage.

In some embodiments, the immune cell is a chimeric antigen receptor (CAR) expressing immune cell. In some embodiments, the CAR is a CAR-T cell. In some embodiments, the CAR is a CAR-NK cell. As used herein, the terms “CAR” refers to an engineered receptor which has specificity for at least one protein of interest (for example a protein expressed by an HVEM expressing cell) and is grafted onto an immune effector cell (such as a T cell or NK cell). In some embodiments, the CAR-T cell has the specificity of a monoclonal antibody grafted onto a T-cell. In some embodiments, the CAR-NK cell has the specificity of a monoclonal antibody grafted onto a NK-cell. In some embodiments, the T cell is selected from a cytotoxic T lymphocyte and a regulatory T cell. MART 1 is an example of a target protein co-expressed with HVEM on target cells. In some embodiments, the CAR targets a protein expressed by the HVEM expressing cells. In some embodiments, the protein is not HVEM. In some embodiments, the CAR targets a protein on the surface of the HVEM expressing cells. In some embodiments, the protein is an antibody. In some embodiments, the CAR targets an antibody. In some embodiments, the CAR targets an antibody of the invention. In some embodiments, the CAR targets a cytotoxic antibody. In some embodiments, the CAR targets an antibody constant domain. In some embodiments, the CAR targets an Fc domain. In some embodiments, the CAR therapy further comprises administering an antibody that targets the HVEM expressing cells. In some embodiments, embodiments, the antibody targeted by the CAR is not the antibody of the invention.

CAR-T and CAR-NK cells and their vectors are well known in the art. Such cells target and are cytotoxic to the protein for which the receptor binds. In some embodiments, a CAR-T or CAR-NK cell targets at least one cancer protein. In some embodiments, a CAR-T or CAR-NK cell targets a plurality of cancer proteins.

Construction of CAR-T cells is well known in the art. In one non-limiting example, a monoclonal antibody to a cancer protein can be made and then a vector coding for the antibody will be constructed. The vector will also comprise a costimulatory signal region. In some embodiments, the costimulatory signal region comprises the intracellular domain of a known T cell or NK cell stimulatory molecule. In some embodiments, the intracellular domain is selected from at least one of the following: CD3Z, CD27, CD28, 4-1BB, OX40, CD30, CD40, PD-1, ICOS, lymphocyte function-associated antigen-1 (LFA-1), CD2, CD 7, LIGHT, NKG2C, B7-H3, and a ligand that specifically binds with CD83. In some embodiments, the vector also comprises a CD3Z signaling domain. This vector is then transfected, for example by lentiviral infection, into a T-cell.

In some embodiments, HVEM is Tumor necrosis factor receptor superfamily member 14 (TNFRSF14). In some embodiments, HVEM is CD270. In some embodiments, HVEM is a receptor of BTLA. In some embodiments, HVEM is a ligand of BTLA. In some embodiments, BTLA is CD272. In some embodiments, HVEM is a receptor of Tumor necrosis factor superfamily member 14 (TNFSF14). In some embodiments, HVEM is a ligand of TNFSF14. In some embodiments, the TNFSF14 is LIGHT. In some embodiments, TNFSF14 is CD258. In some embodiments, HVEM is a receptor of CD160. In some embodiments, HVEM is a ligand of CD160. In some embodiments, HVEM is a receptor of lymphotoxin alpha (LTα). In some embodiments, HVEM is a ligand of LTα. In some embodiments, LTα is TNF-β. In some embodiments, HVEM is a receptor of SALM5. In some embodiments, HVEM is a ligand of SALM5.

In some embodiments, the antibody or antigen binding fragment thereof specifically binds to HVEM. In some embodiments, the antibody or antigen binding fragment thereof binds no other protein other than HVEM. In some embodiments, the antibody or antigen binding fragment thereof binds an extracellular domain of HVEM. In some embodiments, the antibody or antigen binding fragment thereof binds in a ligand binding domain of HVEM. In some embodiments, the antibody or antigen binding fragment thereof binds in a BTLA binding domain of HVEM. In some embodiments, the antibody or antigen binding fragment thereof occludes a BTLA binding domain of HVEM. In some embodiments, the antibody or antigen binding fragment thereof inhibits interaction between HVEM and BTLA. In some embodiments, the antibody or antigen binding fragment thereof blocks interaction between HVEM and BTLA. In some embodiments, the antibody or antigen binding fragment thereof inhibits HVEM mediated, BTLA induced immune suppression. In some embodiments, the antibody or antigen binding fragment thereof binds HVEM and prohibits the bound HVEM from further binding BTLA. In some embodiments, the antibody or antigen binding fragment thereof inhibits downstream signaling through BTLA. In some embodiments, the antibody or antigen binding fragment thereof reduced downstream signaling through BTLA.

In some embodiments, the antibody or antigen binding fragment thereof does not inhibit interaction between HVEM and TNFSF14. In some embodiments, the antibody or antigen binding fragment thereof inhibits interaction between HVEM and TNFSF14. In some embodiments, the TNFSF14 is membranal TNFSF14 (mTNFS14). In some embodiments, the TNFSF14 is soluble TNFSF14 (sTNFS14). In some embodiments, the antibody or antigen binding fragment thereof does not inhibit interaction between HVEM and one of mTNFS14 and sTNFS14 but does inhibit interaction with the other. In some embodiments, the antibody or antigen binding fragment thereof does not inhibit interaction between both of mTNFS14 and sTNFS14 and HVEM. In some embodiments, inhibition is substantial inhibition. In some embodiments, inhibition is at least a 5, 10, 15, 20, 25, 30, 40, 50, 60, 70, 80, 90, 95, 97, 99 or 100% reduction. Each possibility represents a separate embodiment of the invention. In some embodiments, the antibody or antigen binding fragment thereof does not block interaction between HVEM and TNFSF14. In some embodiments, the antibody or antigen binding fragment thereof does not substantially block interaction between HVEM and TNFSF14. In some embodiments, the antibody or antigen binding fragment thereof does not block/inhibit TNFSF14 mediated signaling. In some embodiments, the antibody or antigen binding fragment thereof does not block/inhibit TNFSF14 mediated cell survival.

In some embodiments, the antibody or antigen binding fragment thereof activates signaling through HVEM. In some embodiments, the antibody or antigen binding fragment thereof, induces superior activation as compared to an antibody known in the art. In some embodiments, the antibody or antigen binding fragment thereof is an HVEM agonist. In some embodiments, the antibody or antigen binding fragment thereof, is a superior agonist as compared to an antibody known in the art. In some embodiments, the antibody or antigen binding fragment thereof induces signaling though HVEM. In some embodiments, signaling through HVEM is HVEM downstream signaling. In some embodiments, the antibody or antigen binding fragment thereof binds HVEM on a cell and activates/induces HVEM signaling in the cell. In some embodiments, HVEM signaling comprises activation of nuclear factor kappa-light-chain-enhancer of activated B cells (NF-kB) signaling. In some embodiments, NF-kB signaling comprises control of genes with an NF-kB response element in their promoter. In some embodiments, the signaling comprises increased cytotoxicity of a cell. In some embodiments, the signaling comprises increased cytotoxicity of a cell expressing HVEM contacted by the antibody or antigen binding fragment thereof. In some embodiments, increased cytotoxicity comprises increased secretion of a proinflammatory cytokine. In some embodiments, the proinflammatory cytokine is selected from IL-1, IL-1B, IL-4, IL-6, TNFα, IFNγ, MCP1, IL-12, IL-18, IL-23 and CM-CSF. In some embodiments, the proinflammatory cytokine is IFNγ. In some embodiments, the increase is as compared to a cell not contacted by the antibody or antigen binding fragment thereof of the invention. In some embodiments, the increase is at least a 10, 20, 25, 30, 40, 50, 60, 70, 75, 80, 90, 95, 100, 110, 120, 130, 140, 150, 200, 250, 300, 350, 400, 450, or 500% increase. Each possibility represents a separate embodiment of the invention.

In some embodiments, the cell is an immune cell and the signaling comprises immune activation. In some embodiments, the immune cell is a lymphocyte. In some embodiments, the immune cell is a T cell. In some embodiments, the immune cell is a CD8+ T cell. In some embodiments, the immune cell is a CD4+ T cell. In some embodiments, the immune cell is not a gamma/delta T cell. In some embodiments, the immune cell is a gamma/delta T cell. In some embodiments, the immune cell is an NK cell. In some embodiments, activating HVEM signaling comprises activating the immune cell. In some embodiments, immune activation comprises increased proliferation. In some embodiments, immune activation comprises increased cytotoxicity. In some embodiments, immune activation comprises increased migration. In some embodiments, immune activation comprises increased homing. In some embodiments, immune activation comprises increased cell clustering. In some embodiments, immune activation is T cell activation. In some embodiments, immune activation comprises an increase in the number of Th1 T cells. In some embodiments, the increase is a relative increase as compared to Th2 T cells. In some embodiments, immune activation comprises an increase in CD8+ T cells. In some embodiments, immune activation comprises a decrease in T regulatory cells. In some embodiments, immune activation comprises an increase in the expression of a marker selected from: 41BB, CD69, CD25, CD107a, HLA-DR and secretion of a cytokine. In some embodiments, the cytokine is a proinflammatory cytokine. In some embodiments, T cell activation comprises increased T cell clustering.

In some embodiments, cell is a cancerous cell and the signaling comprises an anti-tumor effect. In some embodiments, the anti-tumor effect comprises increased apoptosis. In some embodiments, the anti-tumor effect comprises decreased proliferation. In some embodiments, the anti-tumor effect comprises increased chemotherapeutic sensitivity. In some embodiments, the anti-tumor effect comprises decreases motility. In some embodiments, the anti-tumor effect comprises decreases invasion. In some embodiments, the anti-tumor effect comprises decreased metastasis. In some embodiments, the anti-tumor effect comprises decreased self-renewal. In some embodiments, the effect is as compared to a cancer cell than was not contacted by the antibody or antigen binding fragment thereof of the invention. In some embodiments, the decrease is at least a 10, 20, 25, 30, 40, 50, 60, 70, 75, 80, 90, 95, 97, 99 or 100% decrease. Each possibility represents a separate embodiment of the invention.

In some embodiments, the antibody or antigen binding fragment thereof does not induce apoptosis. In some embodiments, inducing apoptosis is directly inducing apoptosis. In some embodiments, the antibody or antigen binding fragment thereof does not directly induce apoptosis. As used herein, “direct induction” refers to a result that occurs as an immediate result of binding of the antibody or antigen binding fragment thereof and does not feature downstream signaling within the bound cell. In some embodiments, the antibody or antigen binding fragment thereof is not cytotoxic. In some embodiments, the antibody or antigen binding fragment thereof is not cytotoxic in and of itself. In some embodiments, the antibody or antigen binding fragment thereof does not induce antibody-directed cell cytotoxicity (ADCC). In some embodiments, the antibody or antigen binding fragment thereof does not induce complement dependent cytotoxicity (CDC). In some embodiments, the antibody or antigen binding fragment thereof does not comprise a cytotoxic moiety. In some embodiments, the antibody or antigen binding fragment thereof does not induce apoptosis of a cell expressing HVEM upon binding of the HVEM expressed by the cell. In some embodiments, the antibody or antigen binding fragment thereof does not induce apoptosis via interaction with another cell. In some embodiments, the antibody or antigen binding fragment thereof does not directly induce killing of a cell expressing HVEM. In some embodiments, the antibody or antigen binding fragment thereof does not target a cell for killing. In some embodiments, the antibody or antigen binding fragment thereof does induce indirect apoptosis. In some embodiments, indirect apoptosis is apoptosis induced by downstream signaling through the HVEM receptor that leads to apoptosis. In some embodiments, indirect apoptosis is apoptosis that requires signal transduction. In some embodiments, indirect apoptosis is apoptosis that does not require the involvement of a second cell. In some embodiments, the antibody or antigen binding fragment thereof does not induce apoptosis in an immune cell. In some embodiments, the antibody or antigen binding fragment thereof induces apoptosis in a cancer cell.

In some embodiments, the antibody or antigen binding fragment thereof inhibits interaction between HVEM and CD160. In some embodiments, the antibody or antigen binding fragment thereof, induces superior inhibition as compared to an antibody known in the art. In some embodiments, the antibody or antigen binding fragment thereof inhibits interaction between HVEM and LTα. In some embodiments, the antibody or antigen binding fragment thereof inhibits interaction between HVEM and herpes simplex virus type 1 glycoprotein D (HSV1-gD). In some embodiments, the antibody or antigen binding fragment thereof inhibits interaction between HVEM and at least two of CD160, HSV1-gD and LTα. In some embodiments, the antibody or antigen binding fragment thereof blocks interaction between HVEM and CD160. In some embodiments, the antibody or antigen binding fragment thereof blocks interaction between HVEM and LTα. In some embodiments, the antibody or antigen binding fragment thereof blocks interaction between HVEM and HSV1-gD. In some embodiments, the antibody or antigen binding fragment thereof blocks interaction between HVEM and at least two of CD160, HSV1-gD and LTα. In some embodiments, the antibody or antigen binding fragment thereof inhibits/blocks interaction between HVEM and all of CD160, HSV1-gD and LTα. In some embodiments, the antibody or antigen binding fragment thereof does not inhibit or block interaction between HVEM and at least one of CD160, HSV1-gD and LTα.

In some embodiments, the antibody or fragment thereof is a fab fragment. In some embodiments, the antibody or fragment thereof is a single chain antibody (scFv). In some embodiments, the antibody or fragment thereof is a single domain antibody. In some embodiments, the antibody is a human antibody. In some embodiments, the antibody is a humanized antibody. In some embodiments, the antibody is a monoclonal antibody. In some embodiments, the antibody is a blocking antibody. In some embodiments, the antibody is an affinity matured antibody.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgA1 and IgA2) or subclass. In some embodiments, the antibody comprises IgG2 or IgG4. In some embodiments, the antibody comprises IgG2. In some embodiments, the antibody comprises IgG4. In some embodiments, the antibody comprises IgG1. In some embodiments, the antibody comprises IgG3. In some embodiments, the antibody comprises a modified IgG1 or IgG3 with reduced toxicity.

The basic unit of the naturally occurring antibody structure is a heterotetrameric glycoprotein complex of about 150,000 Daltons, composed of two identical light (L) chains and two identical heavy (H) chains, linked together by both noncovalent associations and by disulfide bonds. Each heavy and light chain also has regularly spaced intra-chain disulfide bridges. Five human antibody classes (IgG, IgA, IgM, IgD and IgE) exist, and within these classes, various subclasses, are recognized based on structural differences, such as the number of immunoglobulin units in a single antibody molecule, the disulfide bridge structure of the individual units, and differences in chain length and sequence. The class and subclass of an antibody is its isotype.

The amino terminal regions of the heavy and light chains are more diverse in sequence than the carboxy terminal regions, and hence are termed the variable domains. This part of the antibody structure confers the antigen-binding specificity of the antibody. A heavy variable (VH) domain and a light variable (VL) domain together form a single antigen-binding site, thus, the basic immunoglobulin unit has two antigen-binding sites. Particular amino acid residues are believed to form an interface between the light and heavy chain variable domains (Chothia et al., J. Mol. Biol. 186, 651-63 (1985); Novotny and Haber, (1985) Proc. Natl. Acad. Sci. USA 82 4592-4596).

The carboxy terminal portion of the heavy and light chains form the constant domains i.e., CH1, CH2, CH3, CL. While there is much less diversity in these domains, there are differences from one animal species to another, and further, within the same individual there are several different isotypes of antibody, each having a different function.

The term “framework region” or “FR” refers to the amino acid residues in the variable domain of an antibody, which are other than the hypervariable region amino acid residues as herein defined. The term “hypervariable region” as used herein refers to the amino acid residues in the variable domain of an antibody, which are responsible for antigen binding. The hypervariable region comprises amino acid residues from a “complementarity determining region” or “CDR”. The CDRs are primarily responsible for binding to an epitope of an antigen. The extent of FRs and CDRs has been precisely defined (see, Kabat et al.). In some embodiments, CDR positions are determined by the Kabat numbering system. In some embodiments, CDR positions are determined by the EU numbering system.

Immunoglobulin variable domains can also be analyzed using the IMGT information system (www://imgt.cines.fr/) (IMGT®/V-Quest) to identify variable region segments, including CDRs. See, e.g., Brochet, X. et al, Nucl. Acids Res. J6:W503-508 (2008).

As used herein, the term “humanized antibody” refers to an antibody from a non-human species whose protein sequences have been modified to increase similarity to human antibodies. A humanized antibody may be produced by production of recombinant DNA coding for the CDRs of the non-human antibody surrounded by sequences that resemble a human antibody. In some embodiments, the humanized antibody is a chimeric antibody. In some embodiments, humanizing comprises insertion of the CDRs of the invention into a human antibody scaffold or backbone. Humanized antibodies are well known in the art and any method of producing them that retains the CDRs of the invention may be employed.

The term “monoclonal antibody” or “mAb” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The modifier “monoclonal” indicates the character of the antibody as being obtained from a substantially homogeneous population of antibodies and is not to be construed as produced by any specific preparation method. Monoclonal antibodies to be used in accordance with the methods provided herein, may be made by the hybridoma method first described by Kohler et al, Nature 256:495 (1975), or may be made by recombinant DNA methods (see, e.g., U.S. Pat. No. 4,816,567). The “monoclonal antibodies” may also be isolated from phage antibody libraries using the techniques described in Clackson et al, Nature 352:624-628 (1991) and Marks et al, J. Mol. Biol. 222:581-597 (1991), for example.

The mAb of the present invention may be of any immunoglobulin class including IgG, IgM, IgD, IgE or IgA. A hybridoma producing a mAb may be cultivated in vitro or in vivo. High titers of mAbs can be obtained in vivo production where cells from the individual hybridomas are injected intraperitoneally into pristine-primed Balb/c mice to produce ascites fluid containing high concentrations of the desired mAbs. mAbs of isotype IgM or IgG may be purified from such ascites fluids, or from culture supernatants, using column chromatography methods well known to those of skill in the art.

“Antibody fragments” comprise a portion of an intact antibody, preferably comprising the antigen binding region thereof. Examples of antibody fragments include Fab, Fab′, F(ab′)2, and Fv fragments; diabodies; tandem diabodies (taDb), linear antibodies (e.g., U.S. Pat. No. 5,641,870, Example 2; Zapata et al, Protein Eng. 8(10): 1057-1062 (1995)); one-armed antibodies, single variable domain antibodies, minibodies, single-chain antibody molecules; multispecific antibodies formed from antibody fragments (e.g., including but not limited to, db-Fc, taDb-Fc, taDb-CH3, (scFV)4-Fc, di-scFv, bi-scFv, or tandem (di,tri)-scFv); and Bi-specific T-cell engagers (BiTEs).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-binding sites and is still capable of cross-linking antigen.

“Fv” is the minimum antibody fragment that contains a complete antigen-recognition and antigen-binding site. This region consists of a dimer of one heavy chain and one light chain variable domain in tight, non-covalent association. It is in this configuration that the three surfaces of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

The Fab fragment also contains the constant domain of the light chain and the first constant domain (CH1) of the heavy chain. Fab′ fragments differ from Fab fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear at least one free thiol group. F(ab′)2 antibody fragments originally were produced as pairs of Fab′ fragments that have hinge cysteines between them. Other chemical couplings of antibody fragments are also known.

The “light chains” of antibodies (immunoglobulins) from any vertebrate species can be assigned to one of two clearly distinct types, called kappa and lambda, based on the amino acid sequences of their constant domains.

Depending on the amino acid sequence of the constant domain of their heavy chains, antibodies can be assigned to different classes. There are five major classes of intact antibodies: IgA, IgD, IgE, IgG, and IgM, and several of these may be further divided into subclasses (isotypes), e.g., IgG1, IgG2, IgG3, IgG4, IgA, and IgA2. The heavy chain constant domains that correspond to the different classes of antibodies are called a, delta, e, gamma, and micro, respectively. The subunit structures and three-dimensional configurations of different classes of immunoglobulins are well known.

“Single-chain Fv” or “scFv” antibody fragments comprise the VH and VL domains of antibody, wherein these domains are present in a single polypeptide chain. In some embodiments, the Fv polypeptide further comprises a polypeptide linker between the VH and VL domains that enables the scFv to form the desired structure for antigen binding. For a review of scFv see Pluckthun in The Pharmacology of Monoclonal Antibodies, vol. 113, Rosenburg and Moore eds., Springer-Verlag, New York, pp. 269-315 (1994).

The term “diabodies” refers to small antibody fragments with two antigen-binding sites, which fragments comprise a heavy chain variable domain (VH) connected to a light chain variable domain (VL) in the same polypeptide chain (VH-VL). By using a linker that is too short to allow pairing between the two domains on the same chain, the domains are forced to pair with the complementary domains of another chain and create two antigen-binding sites. Diabodies production is known in the art and is described in Natl. Acad. Sci. USA, 90:6444-6448 (1993).

The monoclonal antibodies of the invention may be prepared using methods well known in the art. Examples include various techniques, such as those in Kohler, G. and Milstein, C, Nature 256: 495-497 (1975); Kozbor et al, Immunology Today 4: 72 (1983); Cole et al, pg. 77-96 in MONOCLONAL ANTIBODIES AND CANCER THERAPY, Alan R. Liss, Inc. (1985).

Besides the conventional method of raising antibodies in vivo, antibodies can be generated in vitro using phage display technology. Such a production of recombinant antibodies is much faster compared to conventional antibody production and they can be generated against an enormous number of antigens. Furthermore, when using the conventional method, many antigens prove to be non-immunogenic or extremely toxic, and therefore cannot be used to generate antibodies in animals. Moreover, affinity maturation (i.e., increasing the affinity and specificity) of recombinant antibodies is very simple and relatively fast. Finally, large numbers of different antibodies against a specific antigen can be generated in one selection procedure. To generate recombinant monoclonal antibodies, one can use various methods all based on display libraries to generate a large pool of antibodies with different antigen recognition sites. Such a library can be made in several ways: One can generate a synthetic repertoire by cloning synthetic CDR3 regions in a pool of heavy chain germline genes and thus generating a large antibody repertoire, from which recombinant antibody fragments with various specificities can be selected. One can use the lymphocyte pool of humans as starting material for the construction of an antibody library. It is possible to construct naive repertoires of human IgM antibodies and thus create a human library of large diversity. This method has been widely used successfully to select a large number of antibodies against different antigens. Protocols for bacteriophage library construction and selection of recombinant antibodies are provided in the well-known reference text Current Protocols in Immunology, Colligan et al (Eds.), John Wiley & Sons, Inc. (1992-2000), Chapter 17, Section 17.1.

In some embodiments, antibodies and portions thereof include but are not limited to: antibodies, fragments of antibodies, Fab and F(ab′)2, single-domain antigen-binding recombinant fragments and natural nanobodies. In some embodiments, the antigen binding fragment is selected from the group consisting of a Fv, Fab, F(ab′)₂, scFv, scFv₂ or a scFv₄ fragment.

In some embodiments, the present invention provides nucleic acid sequences encoding the antibodies or antigen binding portions of the present invention.

For example, the polynucleotide may encode an entire immunoglobulin molecule chain, such as a light chain or a heavy chain. A complete heavy chain includes not only a heavy chain variable region (VH) but also a heavy chain constant region (CH), which typically will comprise three constant domains: CH1, CH2 and CH3; and a “hinge” region. In some situations, the presence of a constant region is desirable.

Other polypeptides which may be encoded by the polynucleotide include antigen-binding antibody fragments such as single domain antibodies (“dAbs”), Fv, scFv, Fab′ and CH1 and CK or CL domain has been excised. As minibodies are smaller than conventional antibodies they should achieve better tissue penetration in clinical/diagnostic use but being bivalent they should retain higher binding affinity than monovalent antibody fragments, such as dAbs. Accordingly, unless the context dictates otherwise, the term “antibody” as used herein encompasses not only whole antibody molecules, but also antigen-binding antibody fragments of the type discussed above. Each framework region present in the encoded polypeptide may comprise at least one amino acid substitution relative to the corresponding human acceptor framework. Thus, for example, the framework regions may comprise, in total, three, four, five, six, seven, eight, nine, ten, eleven, twelve, thirteen, fourteen, or fifteen amino acid substitutions relative to the acceptor framework regions. Given the properties of the individual amino acids comprising the disclosed protein products, some rational substitutions will be recognized by the skilled worker. Amino acid substitutions, i.e., “conservative substitutions,” may be made, for instance, on the basis of similarity in polarity, charge, solubility, hydrophobicity, hydrophilicity, and/or the amphipathic nature of the residues involved.

Suitably, the polynucleotides described herein may be isolated and/or purified. In some embodiments, the polynucleotides are isolated polynucleotides.

As used herein, the term “non-naturally occurring” substance, composition, entity, and/or any combination of substances, compositions, or entities, or any grammatical variants thereof, is a conditional term that explicitly excludes, but only excludes, those forms of the substance, composition, entity, and/or any combination of substances, compositions, or entities that are well-understood by persons of ordinary skill in the art as being “naturally-occurring,” or that are, or might be at any time, determined or interpreted by a judge or an administrative or judicial body to be, “naturally-occurring”.

In some embodiments, the antibody comprises an IgG4. In some embodiments, the antibody comprises an IgG2. In some embodiments, the antibody comprises an IgG2 or an IgG4. In some embodiments, the antibody comprises an IgG1, IgG2, IgG3 or IgG4. In some embodiments, the antibody does not comprise an IgG1. In some embodiments, the antibody does not comprise an IgG3. In some embodiments, the antibody does not comprise an IgG1 or IgG3. In some embodiments, the antibody comprises a mutated IgG1 and/or IgG3, wherein the mutation inhibits induction of ADCC, CDC or both. In some embodiments, the mutation is in the FcRgamma binding motif.

In some embodiments, the antibody or antigen binding fragment thereof is not an antibody present in WO2020222235. In some embodiments, the antibody or antigen binding fragment thereof does not comprise all of a CDR-H1 comprising the amino acid sequence set forth in SEQ ID NO: 1 (SYAMS), a CDR-H2 comprising the amino acid sequence as set forth in SEQ ID NO: 2 (AISGSGGSTYYADSVKG), a CDR-H3 comprising the amino acid sequence as set forth in SEQ ID NO: 3 (APGDYTAYFDY), a CDR-L1 comprising the amino acid sequence as set forth in SEQ ID NO: 4 (RASQSVSSYLA), a CDR-L2 comprising the amino acid sequence as set forth in SEQ ID NO: 5 (GASSRAT), and a CDR-L3 comprising the amino acid sequence as set forth in SEQ ID NO: 6 (QQYGSSPPYT). In some embodiments, the antibody or antigen binding fragment thereof does not comprise all of a CDR-H2 comprising the amino acid sequence as set forth in SEQ ID NO: 2, a CDR-H3 comprising the amino acid sequence as set forth in SEQ ID NO: 3 and a CDR-L3 comprising the amino acid sequence as set forth in SEQ ID NO: 6. In some embodiments, the antibody or antigen binding fragment thereof does not comprise both of a CDR-H2 comprising the amino acid sequence as set forth in SEQ ID NO: 2, and a CDR-H3 comprising the amino acid sequence as set forth in SEQ ID NO: 3. In some embodiments, the antibody or antigen binding fragment thereof does not comprise a CDR-H2 comprising the amino acid sequence as set forth in SEQ ID NO: 2. In some embodiments, the antibody or antigen binding fragment thereof does not comprise a CDR-H3 comprising the amino acid sequence as set forth in SEQ ID NO: 3. In some embodiments, the antibody or antigen binding fragment thereof does not comprise a CDR-L3 comprising the amino acid sequence as set forth in SEQ ID NO: 6. In some embodiments, the antibody or antigen binding fragment thereof does not comprise all of a CDR-H1 consisting of the amino acid sequence set forth in SEQ ID NO: 1 (SYAMS), a CDR-H2 consisting of the amino acid sequence as set forth in SEQ ID NO: 2 (AISGSGGSTYYADSVKG), a CDR-H3 consisting of the amino acid sequence as set forth in SEQ ID NO: 3 (APGDYTAYFDY), a CDR-L1 consisting of the amino acid sequence as set forth in SEQ ID NO: 4 (RASQSVSSYLA), a CDR-L2 consisting of the amino acid sequence as set forth in SEQ ID NO: 5 (GASSRAT), and a CDR-L3 consisting of the amino acid sequence as set forth in SEQ ID NO: 6 (QQYGSSPPYT). In some embodiments, the antibody or antigen binding fragment thereof does not comprise all of a CDR-H2 consisting of the amino acid sequence as set forth in SEQ ID NO: 2, a CDR-H3 consisting of the amino acid sequence as set forth in SEQ ID NO: 3 and a CDR-L3 consisting of the amino acid sequence as set forth in SEQ ID NO: 6. In some embodiments, the antibody or antigen binding fragment thereof does not comprise both of a CDR-H2 consisting of the amino acid sequence as set forth in SEQ ID NO: 2, and a CDR-H3 consisting of the amino acid sequence as set forth in SEQ ID NO: 3. In some embodiments, the antibody or antigen binding fragment thereof does not comprise a CDR-H2 consisting of the amino acid sequence as set forth in SEQ ID NO: 2. In some embodiments, the antibody or antigen binding fragment thereof does not comprise a CDR-H3 consisting of the amino acid sequence as set forth in SEQ ID NO: 3. In some embodiments, the antibody or antigen binding fragment thereof does not comprise a CDR-L3 consisting of the amino acid sequence as set forth in SEQ ID NO: 6.

In some embodiments, CDR-H2 comprises the amino acid sequence as set forth in SEQ ID NO: 17 (X₁IX₂X₃X₄X₅X₆X₇X₂1YYADSVX₈G) wherein X₁ is A, G or N, X₂ is S, N, G or Y, X₃ is G or S, X₄ is S, N or P, X₅ is G or P, X₆ is G, D, S, E, Q or N, X₇ is S, Y, G or R, X₂₁ is T, A, E, G or N and X₈ is E or K. In some embodiments, CDR-H2 comprises the amino acid sequence as set forth in SEQ ID NO: 49 (X₁IX₂X₃X₄X₅X₁₈X₇X₁₉YYADSVX₂₀G) wherein X₁ is A, G or N, X₂ is S, N, G or Y, X₃ is G or S, X₄ is S, N or P, X₅ is G or P, X₁₈ is any amino acid other than C, X₇ is S, Y, G or R, X₁₉ is any amino acid other than S or C, and X₂₀ is any amino acid.

In some embodiments, X₁₈ is any amino acid other than C. In some embodiments, X₁ₛ is selected from any amino acid other than C. In some embodiments, X₁₈ is X₆. In some embodiments, X₁₈ is G, D, S, or N. In some embodiments, X₁₈ is any amino acid other than G. In some embodiments, X₁₈ is selected from any amino acid other than G.

In some embodiments, X₁₉ is any amino acid other than C. In some embodiments, X₁₉ is selected from any amino acid other than C. In some embodiments, X₁₉ is any amino acid other than S. In some embodiments, X₁₉ is selected from any amino acid other than S. In some embodiments, X₁₉ is any amino acid other than C or S. In some embodiments, X₁₉ is selected from any amino acid other than C and S. In some embodiments, X₁₉ is any amino acid other than T. In some embodiments, X₁₉ is selected from any amino acid other than T. In some embodiments, X₁₉ is T.

In some embodiments, X₂₀ is any amino acid. In some embodiments, X₂₀ is selected from any amino acid. In some embodiments, X₂₀ is an amino acid other than K. In some embodiments, X₂₀ is any amino acid other than C or S. In some embodiments, X₂₀ is an amino acid other than K, C or S. In some embodiments, X₂₀ is a non-positively charged amino acid. In some embodiments, a positively charged amino acid is K, R or H. In some embodiments, a positively charged amino acid is any one of K, R and H. In some embodiments, charged is charged at pH 7.0. In some embodiments, charged is charged at neutral pH. In some embodiments, charged is comprising a charged side chain. In some embodiments, X₂₀ is a negatively charged amino acid. In some embodiments, a negatively charged amino acid is E or D. In some embodiments, a negatively charged amino acid is selected from E and D. In some embodiments, X₂₀ is K or E. In some embodiments, X₂₀ is selected from K and E. In some embodiments, X₂₀ is a negatively charged amino acid or a polar amino acid. In some embodiments, X₂₀ is selected from a negatively charged amino acid and a polar amino acid. In some embodiments, a polar amino acid is Y, S, T, N or Q. In some embodiments, a polar amino acid is selected from Y, S, T, N and Q. In some embodiments, X₂₀ is K. In some embodiments, X₂₀ is E.

In some embodiments, X₁ is A, G or N. In some embodiments, X₁ is selected from A, G and N. In some embodiments, X₂ is S, N, G or Y. In some embodiments, X₂ is selected from S, N, G and Y. In some embodiments, X₃ is G or S. In some embodiments, X₃ is selected from G and S. In some embodiments, X₄ is S, N or P. In some embodiments, X₄ is selected from S, N and P. In some embodiments, X₅ is G or P. In some embodiments, X₅ is selected from G and P. In some embodiments, X₆ is G, D, S, or N. In some embodiments, X₆ is selected from G, D, S, and N. In some embodiments, X₇ is S, Y, G or R. In some embodiments, X₇ is selected from S, Y, G and R. In some embodiments, X₈ is E or K. In some embodiments, X₈ is selected from E and K.

In some embodiments, CDR-H2 comprises an amino acid sequence selected from:

SEQ ID NO: 2, SEQ ID NO: 20
(GINGNGDYTYYADSVKG),
SEQ ID NO: 21
(AIGGSGSGTYYADSVKG),
SEQ ID NO: 22
(NIYSNPNRTYYADSVEG)
SEQ ID NO: 23
(NINGPGNGTYYADSVEG),
SEQ ID NO: 47
(NIYSNPNRTYYADSVKG),
SEQ ID NO: 48
(AISGSGGSTYYADSVEG),
SEQ ID NO: 57
(NIYSNPDRTYYADSVEG),
SEQ ID NO: 58
(NIYSNPERTYYADSVEG),
SEQ ID NO: 59
(NIYSNPGRTYYADSVEG),
SEQ ID NO: 60
(NIYSNPQRTYYADSVEG),
SEQ ID NO: 61
(NIYSNPSRTYYADSVEG),
SEQ ID NO: 62
(NIYSNPNRAYYADSVEG),
SEQ ID NO: 63
(NIYSNPNREYYADSVEG),
SEQ ID NO: 64
(NIYSNPNRGYYADSVEG)
and
SEQ ID NO: 65
(NIYSNPNRNYYADSVEG).

In some embodiments, CDR-H2 comprises SEQ ID NO: 2. In some embodiments, CDR-H2 consists of SEQ ID NO: 2. In some embodiments, CDR-H2 comprises SEQ ID NO: 20. In some embodiments, CDR-H2 consists of SEQ ID NO: 20. In some embodiments, CDR-H2 comprises SEQ ID NO: 21. In some embodiments, CDR-H2 consists of SEQ ID NO: 21. In some embodiments, CDR-H2 comprises SEQ ID NO: 22. In some embodiments, CDR-H2 consists of SEQ ID NO: 22. In some embodiments, CDR-H2 comprises SEQ ID NO: 23. In some embodiments, CDR-H2 consists of SEQ ID NO: 23. In some embodiments, CDR-H2 is SEQ ID NO: 17. In some embodiments, CDR-H2 comprises SEQ ID NO: 17. In some embodiments, CDR-H2 consists of SEQ ID NO: 17. In some embodiments, CDR-H2 comprises SEQ ID NO: 47. In some embodiments, CDR-H2 consists of SEQ ID NO: 47. In some embodiments, CDR-H2 comprises SEQ ID NO: 48. In some embodiments, CDR-H2 consists of SEQ ID NO: 48. In some embodiments, CDR-H2 comprises SEQ ID NO: 57. In some embodiments, CDR-H2 consists of SEQ ID NO: 57. In some embodiments, CDR-H2 comprises SEQ ID NO: 58. In some embodiments, CDR-H2 consists of SEQ ID NO: 58. In some embodiments, CDR-H2 comprises SEQ ID NO: 59. In some embodiments, CDR-H2 consists of SEQ ID NO: 59. In some embodiments, CDR-H2 comprises SEQ ID NO: 60. In some embodiments, CDR-H2 consists of SEQ ID NO: 60. In some embodiments, CDR-H2 comprises SEQ ID NO: 61. In some embodiments, CDR-H2 consists of SEQ ID NO: 61. In some embodiments, CDR-H2 comprises SEQ ID NO: 62. In some embodiments, CDR-H2 consists of SEQ ID NO: 62. In some embodiments, CDR-H2 comprises SEQ ID NO: 63. In some embodiments, CDR-H2 consists of SEQ ID NO: 63. In some embodiments, CDR-H2 comprises SEQ ID NO: 64. In some embodiments, CDR-H2 consists of SEQ ID NO: 64. In some embodiments, CDR-H2 comprises SEQ ID NO: 65. In some embodiments, CDR-H2 consists of SEQ ID NO: 65.

In some embodiments, CDR-H3 comprises an amino acid sequence selected from: SEQ ID NO: 3, SEQ ID NO: 24 (ASYRNYNYGDY), SEQ ID NO: 25 (ASYDPTNYYDY) and SEQ ID NO: 26 (ASYRSTNYFDY). In some embodiments, CDR-H3 comprises SEQ ID NO: 3. In some embodiments, CDR-H3 consists of SEQ ID NO: 3. In some embodiments, CDR-H3 comprises SEQ ID NO: 24. In some embodiments, CDR-H3 consists of SEQ ID NO: 24. In some embodiments, CDR-H3 comprises SEQ ID NO: 25. In some embodiments, CDR-H3 consists of SEQ ID NO: 25. In some embodiments, CDR-H3 comprises SEQ ID NO: 26. In some embodiments, CDR-H3 consists of SEQ ID NO: 26. In some embodiments, CDR-H3 is SEQ ID NO: 18. In some embodiments, CDR-H2 comprises SEQ ID NO: 18. In some embodiments, CDR-H2 consists of SEQ ID NO: 18.

In some embodiments, CDR-L3 comprises an amino acid sequence selected from: SEQ ID NO: 6 and SEQ ID NO: 27 (QQYGSYPPLT). In some embodiments, CDR-L3 comprises SEQ ID NO: 6. In some embodiments, CDR-L3 consists of SEQ ID NO: 6. In some embodiments, CDR-L3 comprises SEQ ID NO: 27. In some embodiments, CDR-L3 consists of SEQ ID NO: 27. In some embodiments, CDR-L3 is SEQ ID NO: 19. In some embodiments, CDR-H2 comprises SEQ ID NO: 19. In some embodiments, CDR-H2 consists of SEQ ID NO: 19.

In some embodiments, a CDR is devoid of a labile liability. In some embodiments, the CDRs are devoid of a labile liability. In some embodiments, the labile liability is an acid labile liability. In some embodiments, the liability is liability to degradation. In some embodiments, the liability is liability to cleavage. In some embodiments, the liability is a DP di-amino acid. In some embodiments, the X₁₁X₁₂ is not DP. In some embodiments, if X₁₁ is D, X₁₂ is not P, G, D, S, T or H. In some embodiments, if X₁₁ is D, X₁₂ is not P. In some embodiments, the liability is a liability to aspartate isomerization. In some embodiments, the liability is a DG di-amino acid. In some embodiments, the liability is a DT di-amino acid. In some embodiments, the liability is a DS di-amino acid. In some embodiments, the liability is a DD di-amino acid. In some embodiments, the liability is a DH di-amino acid. In some embodiments, the liability is a liability to deamination. In some embodiments, the liability is a NG di-amino acid. In some embodiments, the liability is a NS di-amino acid. In some embodiments, the liability is a NT di-amino acid. In some embodiments, the liability is a NH di-amino acid. In some embodiments, the liability is a NN di-amino acid. In some embodiments, the liability is a liability to N-linked glycosylation. In some embodiments, the liability is NXS/T (SEQ ID NO: 84). In some embodiments, the liability is NXS. In some embodiments, the liability is NXT. It will be understood that the X in these liability sequences may be any amino acid. In some embodiments, the liability is a free cysteine residue. In some embodiments, all CDRs are devoid of a free cysteine residue. In some embodiments, the liability is an oxidation liability. In some embodiments, the liability is a surface exposed methionine or tryptophan residue. In some embodiments, the liability is a surface exposed methionine residue. In some embodiments, the liability is a surface exposed tryptophan residue. In some embodiments, the CDR comprises a single amino acid change to abolish the liability. In some embodiments, the CDR comprises a single amino acid change to abolish the di-amino acid sequence.

In some embodiments, CDR-H2 is devoid of an N-glycosylation site. In some embodiments, an N-glycosylation site comprises NXS/T wherein X is any amino acid and wherein the third amino acid is S or T. In some embodiments, CDR-H2 comprises SEQ ID NO: 106 (NIYSNPNRX₂₂YYADSVEG). In some embodiments, X₂₂ is any amino acid other than S and T. In some embodiments, CDR-H2 comprises SEQ ID NO: 107 (NIYSNPNRX₂₃YYADSVEG). In some embodiments, X₂₃ is any amino acid other than S, T and C. In some embodiments, CDR-H2 consists of SEQ ID NO: 106. In some embodiments, CDR-H2 consists of SEQ ID NO: 107. In some embodiments, CDR-H2 comprises SEQ ID NO: 112 (NIYSNPX₂₄RTYYADSVEG). In some embodiments, X₂₄ is any amino acid other than N. In some embodiments, CDR-H2 comprises SEQ ID NO: 113 (NIYSNPX₂₅RTYYADSVEG). In some embodiments, X₂₅ is any amino acid other than N and C. In some embodiments, CDR-H2 comprises SEQ ID NO: 114 (NIYSNPX₂₆RTYYADSVEG). In some embodiments, X₂₆ is G, D, S, E, Q or N. In some embodiments, CDR-H2 consists of SEQ ID NO: 112. In some embodiments, CDR-H2 consists of SEQ ID NO: 113. In some embodiments, CDR-H2 consists of SEQ ID NO: 114.

In some embodiments, CDR-H1 comprises the amino acid sequence set forth in SEQ ID NO: 1, CDR-H2 comprises an amino acid sequence selected from SEQ ID NO: 20, SEQ ID NO: 21, SEQ ID NO: 22, SEQ ID NO: 23, SEQ ID NO: 47, SEQ ID NO: 48, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64 and SEQ ID NO: 65, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 20, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 21, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 22, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 23, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 47, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 48, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 57, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 58, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 59, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 60, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 61, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 62, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 63, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 64, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 65, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6.

In some embodiments, CDR-H1 comprises the amino acid sequence set forth in SEQ ID NO: 1, CDR-H2 comprises the amino acid sequence set forth in SEQ ID NO: 2, CDR-H3 comprises an amino acid sequence selected from SEQ ID NO: 24, SEQ ID NO: 25 and SEQ ID NO: 26, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 2, CDR-H3 comprises the amino acid sequence set forth in SEQ ID NO: 24, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 2, CDR-H3 comprises the amino acid sequence set forth in SEQ ID NO: 25, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6. In some embodiments, CDR-H1 comprises the amino acid sequence set forth in SEQ ID NO: 1, CDR-H2 comprises the amino acid sequence set forth in SEQ ID NO: 2, CDR-H3 comprises the amino acid sequence set forth in SEQ ID NO: 26, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6.

In some embodiments, CDR-H1 comprises the amino acid sequence set forth in SEQ ID NO: 1, CDR-H2 comprises the amino acid sequence set forth in SEQ ID NO: 2, CDR-H3 comprises the amino acid sequence set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 27.

In some embodiments, CDR-H1 comprises the amino acid sequence set forth in SEQ ID NO: 1, CDR-H2 comprises an amino acid sequence selected from SEQ ID NO: 2, SEQ ID NO: 20, SEQ ID NO: 21, SEQ ID NO: 22 and SEQ ID NO: 23, CDR-H3 comprises an amino acid sequence selected from SEQ ID NO: 3, SEQ ID NO: 24, SEQ ID NO: 25 and SEQ ID NO: 26, CDR-L1 comprises the amino acid sequence as set forth in SEQ ID NO: 4, CDR-L2 comprises the amino acid sequence as set forth in SEQ ID NO: 5, and CDR-L3 comprises an amino acid sequence selected from SEQ ID NO: 6 and SEQ ID NO: 27.

In some embodiments, the antibody or antigen binding fragment thereof does not comprise a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKAPGDYTAYFD YWGQGTLVTVSS (SEQ ID NO: 7). In some embodiments, the antibody or antigen binding fragment thereof does not comprise a heavy chain variable region comprising or consisting of SEQ ID NO: 7. In some embodiments, the antibody or antigen binding fragment thereof comprises a heavy chain comprising SEQ ID NO:7 and a light chain comprising a CDR-L3 comprising SEQ ID NO: 27.

In some embodiments, the antibody or antigen binding fragment thereof comprises a heavy chain comprising QVQLVQSGGGLVQPGGSLRLSCAASGFTFSSYAMSWVRQAPGKGLEWVSGINGN GDYTYYADSVKGRFTISRDNSKNTLYLQMNSLRAEDTAVYYCAKAPGDYTAYFD YWGQGTLVTVSS (SEQ ID NO: 28). In some embodiments, the antibody or antigen binding fragment thereof comprises a heavy chain comprising QVQLVQSGGGLVQPGGSLRLSCAASGFTFSSYAMSWVRQAPGKGLEWVSAIGGS GSGTYYADSVKGRFTISRDNSKNTLYLQMNSLRAEDTAVYYCAKAPGDYTAYFD YWGQGTLVTVSS (SEQ ID NO: 29). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRTYYADSVEGRFTISRDNSKNTLYLQMNSLRAEDTAVYYCAKAPGDYTAYFD YWGQGTLVTVSS (SEQ ID NO: 30). In some embodiments, the antibody or antigen binding fragment thereof comprises a heavy chain comprising QVQLVQSGGGLVQPGGSLRLSCAASGFTFSSYAMSWVRQAPGKGLEWVSNINGP GNGTYYADSVEGRFTISRDNSKNTLYLQMNSLRAEDTAVYYCAKAPGDYTAYFD YWGQGTLVTVSS (SEQ ID NO: 31). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRTYYADSVKGRFTISRDNSKNTLYLQMNSLRAEDTAVYYCAKAPGDYTAYFD YWGQGTLVTVSS (SEQ ID NO: 50). In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVEGRFTISRDNSKNTLYLQMNSLRAEDTAVYYCAKAPGDYTAYFD YWGQGTLVTVSS (SEQ ID NO: 51). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PDRTYYADSVEGRFTISRDNSKNTLYLQMNSLRAEDTAVYYCAKAPGDYTAYFD YWGQGTLVTVSS (SEQ ID NO: 66). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PERTYYADSVEGRFTISRDNSKNTLYLQMNSLRAEDTAVYYCAKAPGDYTAYFD YWGQGTLVTVSS (SEQ ID NO: 67). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PGRTYYADSVEGRFTISRDNSKNTLYLQMNSLRAEDTAVYYCAKAPGDYTAYFD YWGQGTLVTVSS (SEQ ID NO: 68). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PQRTYYADSVEGRFTISRDNSKNTLYLQMNSLRAEDTAVYYCAKAPGDYTAYFD YWGQGTLVTVSS (SEQ ID NO: 69). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PSRTYYADSVEGRFTISRDNSKNTLYLQMNSLRAEDTAVYYCAKAPGDYTAYFDY WGQGTLVTVSS (SEQ ID NO: 70). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRAYYADSVEGRFTISRDNSKNTLYLQMNSLRAEDTAVYYCAKAPGDYTAYFD YWGQGTLVTVSS (SEQ ID NO: 71). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNREYYADSVEGRFTISRDNSKNTLYLQMNSLRAEDTAVYYCAKAPGDYTAYFD YWGQGTLVTVSS (SEQ ID NO: 72). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRGYYADSVEGRFTISRDNSKNTLYLQMNSLRAEDTAVYYCAKAPGDYTAYFD YWGQGTLVTVSS (SEQ ID NO: 73). In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRNYYADSVEGRFTISRDNSKNTLYLQMNSLRAEDTAVYYCAKAPGDYTAYFD YWGQGTLVTVSS (SEQ ID NO: 74). In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKASYRNYNYGD YWGQGTLVTVSS (SEQ ID NO: 32). In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKASYDPTNYYD YWGQGTLVTVSS (SEQ ID NO: 33). In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKASYRSTNYFD YWGQGTLVTVSS (SEQ ID NO: 34). In some embodiments, the antibody or antigen binding fragment comprises a heavy chain comprising a sequence selected from SEQ ID NO: 28-34, 50-51 and 66-74. In some embodiments, the antibody or antigen binding fragment comprises a heavy chain comprising a sequence selected from SEQ ID NO: 7, 28-34, 50-51 and 66-74. In some embodiments, the antibody or antigen binding fragment comprises a heavy chain variable region comprising a sequence selected from SEQ ID NO: 28-34, 50-51 and 66-74. In some embodiments, the antibody or antigen binding fragment comprises a heavy chain variable region comprising a sequence selected from SEQ ID NO: 7, 28-34, 50-51 and 66-74. In some embodiments, the antibody or antigen binding fragment comprises a heavy chain variable region consisting of a sequence selected from SEQ ID NO: 28-34, 50-51 and 66-74. In some embodiments, the antibody or antigen binding fragment comprises a heavy chain variable region consisting of a sequence selected from SEQ ID NO: 7, 28-34, 50-51 and 66-74.

In some embodiments, the antibody or antigen binding fragment comprises a light chain comprising a sequence selected from ELVLTQSPATLSLSPGERATLSCRASQSVSSYLAWYQQKPGQAPRLLIYGASSRAT GIPDRFSGSGSGTDFTLTISRLEPEDFAVYYCQQYGSSPPYTFGQGTKVEIK (SEQ ID NO: 8) and ELVLTQSPATLSLSPGERATLSCRASQSVSSYLAWYQQKPGQAPRLLIYGASSRAT GIPDRFSGSGSGTDFTLTISRLEPEDFAVYYCQQYGSYPPLTFGQGTKVEIK (SEQ ID NO: 35). In some embodiments, the antibody or antigen binding fragment comprises a light chain variable region comprising or consisting of a sequence selected from SEQ ID NO: 8 and SEQ ID NO: 35. In some embodiments, the antibody or antigen binding fragment comprises a light chain comprising SEQ ID NO: 8. In some embodiments, the antibody or antigen binding fragment comprises a light chain comprising SEQ ID NO: 35. In some embodiments, the antibody or antigen binding fragment comprises a light chain comprising a sequence selected from SEQ ID NO: 8 and 35. In some embodiments, the antibody or antigen binding fragment comprises a light chain variable region comprising SEQ ID NO: 35. In some embodiments, the antibody or antigen binding fragment comprises a light chain variable region comprising a sequence selected from SEQ ID NO: 8 and 35. In some embodiments, the antibody or antigen binding fragment comprises a light chain variable region consisting of SEQ ID NO: 35. In some embodiments, the antibody or antigen binding fragment comprises a light chain variable region consisting of a sequence selected from SEQ ID NO: 8 and 35.

In some embodiments, the heavy chain comprises an N-terminal peptide comprising MGWSCIILFLVATATGVHS (SEQ ID NO: 36). In some embodiments, the heavy chain comprises an N-terminal peptide consisting of SEQ ID NO: 36. In some embodiments, the heavy chain comprises an amino acid sequence of SEQ ID NO: 36. In some embodiments, the heavy chain comprises an amino acid sequence of SEQ ID NO: 36 N-terminal to any one of SEQ ID NO: 7, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 50, SEQ ID NO: 51, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73 and SEQ ID NO: 74. In some embodiments, the N-terminal peptide is a signal peptide. In some embodiments, the heavy chain comprises a signal peptide. In some embodiments, the heavy chain is devoid of a signal peptide. In some embodiments, the heavy chain lacks a signal peptide. In some embodiments, the heavy chain comprises a signal peptide when first expressed in a cell and the signal peptide is cleaved and the secreted heavy chain lacks the signal peptide. Signal peptides are well known in the art and any signal peptide that is functional to lead to secretion of the antibody chains may be used.

In some embodiments, the heavy chain comprises a C-terminal peptide comprising ASTKGPSVFPLAPCSRSTSESTAALGCLVKDYFPEPVTVSWNSGALTSGVHTFPAV LQSSGLYSLSSVVTVPSSSLGTKTYTCNVDHKPSNTKVDKRVESKYGPPCPPCPAP EFLGGPSVFLFPPKPKDTLMISRTPEVTCVVVDVSQEDPEVQFNWYVDGVEVHNA KTKPREEQFNSTYRVVSVLTVLHQDWLNGKEYKCKVSNKGLPSSIEKTISKAKGQ PREPQVYTLPPSQEEMTKNQVSLTCLVKGFYPSDIAVEWESNGQPENNYKTTPPVL DSDGSFFLYSRLTVDKSRWQEGNVFSCSVMHEALHNHYTQKSLSLSLGK (SEQ ID NO: 37). In some embodiments, the heavy chain comprises a C-terminal peptide consisting of SEQ ID NO: 37. In some embodiments, the C-terminal peptide is a heavy chain constant region. In some embodiments, the heavy chain comprises an amino acid sequence of SEQ ID NO: 37. In some embodiments, the heavy chain comprises an amino acid sequence of SEQ ID NO: 37 C-terminal to any one of SEQ ID NO: 7, SEQ ID NO: 28, SEQ ID NO: 29, SEQ ID NO: 30, SEQ ID NO: 31, SEQ ID NO: 32, SEQ ID NO: 33, SEQ ID NO: 34, SEQ ID NO: 50, SEQ ID NO: 51, SEQ ID NO: 66, SEQ ID NO: 67, SEQ ID NO: 68, SEQ ID NO: 69, SEQ ID NO: 70, SEQ ID NO: 71, SEQ ID NO: 72, SEQ ID NO: 73 and SEQ ID NO: 74. In some embodiments, the C-terminal peptide is an IgG backbone. In some embodiments, the antigen binding fragment lacks a C-terminal peptide. In some embodiments, the antigen binding fragment is devoid of a C-terminal peptide. In some embodiments, the C-terminal peptide is not cytotoxic. In some embodiments, the C-terminal peptide is mutated to reduce cytotoxicity. In some embodiments, the C-terminal peptide comprises an antibody hinge domain. It will be understood by a skilled artisan that the disulfide bridges present in the hinge domain of the heavy chains are sufficient for heavy chain dimerization. The hinge region itself does not confer cytotoxicity. In some embodiments, the C-terminal peptide comprises a CH1 domain. It will be understood by a skilled artisan that the disulfide bridge that forms between the CH1 domain of the heavy chain and the CL domain of the light chain are sufficient for heavy chain-light chain dimerization and formation of a function antibody or antigen binding fragment. In some embodiments, the C-terminal peptide comprises a dimerization domain. In some embodiments, the dimerization domain is sufficient to induce dimerization between the two heavy chains. In some embodiments, the dimerization domain is sufficient to induce dimerization between the heavy chain and the light chain. In some embodiments, the C-terminal peptide comprises two dimerization domains, a first dimerization domain that is sufficient to induce dimerization between the two heavy chains and a second dimerization domain that is sufficient to induce dimerization between the heavy and light chain. In some embodiments, dimerizing is forming a disulfide bond.

In some embodiments, the antibody or antigen binding fragment thereof comprises a heavy chain comprising MGWSCIILFLVATATGVHSQVQLVQSGGGLVQPGGSLRLSCAASGFTFSSYAMSW VRQAPGKGLEWVSAISGSGGSTYYADSVKGRFTISRDNSKNTLYLQMNSLRAEDT AVYYCAKAPGDYTAYFDYWGQGTLVTVSSASTKGPSVFPLAPCSRSTSESTAALG CLVKDYFPEPVTVSWNSGALTSGVHTFPAVLQSSGLYSLSSVVTVPSSSLGTKTYT CNVDHKPSNTKVDKRVESKYGPPCPPCPAPEFLGGPSVFLFPPKPKDTLMISRTPEV TCVVVDVSQEDPEVQFNWYVDGVEVHNAKTKPREEQFNSTYRVVSVLTVLHQD WLNGKEYKCKVSNKGLPSSIEKTISKAKGQPREPQVYTLPPSQEEMTKNQVSLTCL VKGFYPSDIAVEWESNGQPENNYKTTPPVLDSDGSFFLYSRLTVDKSRWQEGNVF SCSVMHEALHNHYTQKSLSLSLGK (SEQ ID NO: 9). In some embodiments, the heavy chain consists of the sequence of SEQ ID NO: 9. It will be understood that SEQ ID NO: 9 can also lack the signal peptide (SEQ ID NO: 36) which produces QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 85) or can contain a different signal peptide.

In some embodiments, the antibody or antigen binding fragment thereof comprises a heavy chain comprising QVQLVQSGGGLVQPGGSLRLSCAASGFTFSSYAMSWVRQAPGKGLEWVSX₁IX₂X₃X₄X₅X₆X₇TYYADSVX₈GRFTISRDNSKNTLYLQMNSLRAEDTAVYYCAKAPGDYT AYFDYWGQGTLVTVSSASTKGPSVFPLAPCSRSTSESTAALGCLVKDYFPEPVTVS WNSGALTSGVHTFPAVLQSSGLYSLSSVVTVPSSSLGTKTYTCNVDHKPSNTKVD KRVESKYGPPCPPCPAPEFLGGPSVFLFPPKPKDTLMISRTPEVTCVVVDVSQEDPE VQFNWYVDGVEVHNAKTKPREEQFNSTYRVVSVLTVLHQDWLNGKEYKCKVSN KGLPSSIEKTISKAKGQPREPQVYTLPPSQEEMTKNQVSLTCLVKGFYPSDIAVEWE SNGQPENNYKTTPPVLDSDGSFFLYSRLTVDKSRWQEGNVFSCSVMHEALHNHYT QKSLSLSLGK (SEQ ID NO: 54). In some embodiments, the heavy chain consists of the sequence of SEQ ID NO: 54. It will be understood that SEQ ID NO: 54 can also include the signal peptide of the invention (SEQ ID NO: 36) or contain a different signal peptide.

In some embodiments, the antibody or antigen binding fragment thereof comprises a heavy chain comprising QVQLVQSGGGLVQPGGSLRLSCAASGFTFSSYAMSWVRQAPGKGLEWVSX₁IX₂X₃X₄X₅X₁₈X₇X₁₉YYADSVX₂₀GRFTISRDNSKNTLYLQMNSLRAEDTAVYYCAKAPGD YTAYFDYWGQGTLVTVSSASTKGPSVFPLAPCSRSTSESTAALGCLVKDYFPEPVT VSWNSGALTSGVHTFPAVLQSSGLYSLSSVVTVPSSSLGTKTYTCNVDHKPSNTKV DKRVESKYGPPCPPCPAPEFLGGPSVFLFPPKPKDTLMISRTPEVTCVVVDVSQEDP EVQFNWYVDGVEVHNAKTKPREEQFNSTYRVVSVLTVLHQDWLNGKEYKCKVS NKGLPSSIEKTISKAKGQPREPQVYTLPPSQEEMTKNQVSLTCLVKGFYPSDIAVE WESNGQPENNYKTTPPVLDSDGSFFLYSRLTVDKSRWQEGNVFSCSVMHEALHN HYTQKSLSLSLGK (SEQ ID NO: 55). In some embodiments, the heavy chain consists of the sequence of SEQ ID NO: 55. It will be understood that SEQ ID NO: 55 can also include the signal peptide of the invention (SEQ ID NO: 36) or contain a different signal peptide.

In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKAX₉X₁₀X₁₁X₁₂X₁₃X₁₄YX₁₅DYWGQGTLVTVSSASTKGPSVFPLAPCSRSTSESTAALGCLVKDYFPEPV TVSWNSGALTSGVHTFPAVLQSSGLYSLSSVVTVPSSSLGTKTYTCNVDHKPSNTK VDKRVESKYGPPCPPCPAPEFLGGPSVFLFPPKPKDTLMISRTPEVTCVVVDVSQED PEVQFNWYVDGVEVHNAKTKPREEQFNSTYRVVSVLTVLHQDWLNGKEYKCKV SNKGLPSSIEKTISKAKGQPREPQVYTLPPSQEEMTKNQVSLTCLVKGFYPSDIAVE WESNGQPENNYKTTPPVLDSDGSFFLYSRLTVDKSRWQEGNVFSCSVMHEALHN HYTQKSLSLSLGK (SEQ ID NO: 56). In some embodiments, the heavy chain consists of the sequence of SEQ ID NO: 56. It will be understood that SEQ ID NO: 56 can also include the signal peptide of the invention (SEQ ID NO: 36) or contain a different signal peptide.

In some embodiments, the antibody or antigen binding fragment thereof comprises a heavy chain comprising QVQLVQSGGGLVQPGGSLRLSCAASGFTFSSYAMSWVRQAPGKGLEWVSGINGN GDYTYYADSVKGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 86). It will be understood that SEQ ID NO: 86 can also include the signal peptide of the invention (SEQ ID NO: 36) producing SEQ ID NO: 39 or contain a different signal peptide. In some embodiments, the antibody or antigen binding fragment thereof comprises a heavy chain consisting of SEQ ID NO: 86 or 39. In some embodiments, the antibody or antigen binding fragment thereof comprises a heavy chain comprising QVQLVQSGGGLVQPGGSLRLSCAASGFTFSSYAMSWVRQAPGKGLEWVSAIGGS GSGTYYADSVKGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 87). It will be understood that SEQ ID NO: 87 can also include the signal peptide of the invention (SEQ ID NO: 36) producing SEQ ID NO: 40 or contain a different signal peptide. In some embodiments, the antibody or antigen binding fragment thereof comprises a heavy chain consisting of SEQ ID NO: 87 or 40. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 88). It will be understood that SEQ ID NO: 88 can also include the signal peptide of the invention (SEQ ID NO: 36) producing SEQ ID NO: 41 or contain a different signal peptide. In some embodiments, the antibody or antigen binding fragment thereof comprises a heavy chain consisting of SEQ ID NO: 88 or 41. In some embodiments, the antibody or antigen binding fragment thereof comprises a heavy chain comprising QVQLVQSGGGLVQPGGSLRLSCAASGFTFSSYAMSWVRQAPGKGLEWVSNINGP GNGTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 89). It will be understood that SEQ ID NO: 89 can also include the signal peptide of the invention (SEQ ID NO: 36) producing SEQ ID NO: 42 or contain a different signal peptide. In some embodiments, the antibody or antigen binding fragment thereof comprises a heavy chain consisting of SEQ ID NO: 89 or 42. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRTYYADSVKGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 90). It will be understood that SEQ ID NO: 90 can also include the signal peptide of the invention (SEQ ID NO: 36) producing SEQ ID NO: 52 or contain a different signal peptide. In some embodiments, the antibody or antigen binding fragment thereof comprises a heavy chain consisting of SEQ ID NO: 90 or 52. In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 91). It will be understood that SEQ ID NO: 91 can also include the signal peptide of the invention (SEQ ID NO: 36) producing SEQ ID NO: 53 or contain a different signal peptide. In some embodiments, the antibody or antigen binding fragment thereof comprises a heavy chain consisting of SEQ ID NO: 91 or 53. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PDRTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 92). It will be understood that SEQ ID NO: 92 can also include the signal peptide of the invention (SEQ ID NO: 36) producing SEQ ID NO: 75 or contain a different signal peptide. In some embodiments, the antibody or antigen binding fragment thereof comprises a heavy chain consisting of SEQ ID NO: 92 or 75. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PERTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 93). It will be understood that SEQ ID NO: 93 can also include the signal peptide of the invention (SEQ ID NO: 36) producing SEQ ID NO: 76 or contain a different signal peptide. In some embodiments, the antibody or antigen binding fragment thereof comprises a heavy chain consisting of SEQ ID NO: 93 or 76. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PGRTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 94). It will be understood that SEQ ID NO: 94 can also include the signal peptide of the invention (SEQ ID NO: 36) producing SEQ ID NO: 77 or contain a different signal peptide. In some embodiments, the antibody or antigen binding fragment thereof comprises a heavy chain consisting of SEQ ID NO: 94 or 77. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PQRTYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 95). It will be understood that SEQ ID NO: 95 can also include the signal peptide of the invention (SEQ ID NO: 36) producing SEQ ID NO: 78 or contain a different signal peptide. In some embodiments, the antibody or antigen binding fragment thereof comprises a heavy chain consisting of SEQ ID NO: 95 or 78. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PSRTYYADSVEGRFTISRDNSKNTLYLQMNSLRAEDTAVYYCAKAPGDYTAYFDY WGQGTLVTVSSASTKGPSVFPLAPCSRSTSESTAALGCLVKDYFPEPVTVSWNSGA LTSGVHTFPAVLQSSGLYSLSSVVTVPSSSLGTKTYTCNVDHKPSNTKVDKRVESK YGPPCPPCPAPEFLGGPSVFLFPPKPKDTLMISRTPEVTCVVVDVSQEDPEVQFNW YVDGVEVHNAKTKPREEQFNSTYRVVSVLTVLHQDWLNGKEYKCKVSNKGLPSS IEKTISKAKGQPREPQVYTLPPSQEEMTKNQVSLTCLVKGFYPSDIAVEWESNGQP ENNYKTTPPVLDSDGSFFLYSRLTVDKSRWQEGNVFSCSVMHEALHNHYTQKSLS LSLGK (SEQ ID NO: 96). It will be understood that SEQ ID NO: 96 can also include the signal peptide of the invention (SEQ ID NO: 36) producing SEQ ID NO: 79 or contain a different signal peptide. In some embodiments, the antibody or antigen binding fragment thereof comprises a heavy chain consisting of SEQ ID NO: 96 or 79. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRAYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 97). It will be understood that SEQ ID NO: 97 can also include the signal peptide of the invention (SEQ ID NO: 36) producing SEQ ID NO: 80 or contain a different signal peptide. In some embodiments, the antibody or antigen binding fragment thereof comprises a heavy chain consisting of SEQ ID NO: 97 or 80. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNREYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 98). It will be understood that SEQ ID NO: 98 can also include the signal peptide of the invention (SEQ ID NO: 36) producing SEQ ID NO: 81 or contain a different signal peptide. In some embodiments, the antibody or antigen binding fragment thereof comprises a heavy chain consisting of SEQ ID NO: 98 or 81. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRGYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 99). It will be understood that SEQ ID NO: 99 can also include the signal peptide of the invention (SEQ ID NO: 36) producing SEQ ID NO: 82 or contain a different signal peptide. In some embodiments, the antibody or antigen binding fragment thereof comprises a heavy chain consisting of SEQ ID NO: 99 or 82. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRNYYADSVEGRFTISRDNSKNTLYLQMNSLRAEDTAVYYCAKAPGDYTAYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 100). It will be understood that SEQ ID NO: 100 can also include the signal peptide of the invention (SEQ ID NO: 36) producing SEQ ID NO: 83 or contain a different signal peptide. In some embodiments, the antibody or antigen binding fragment thereof comprises a heavy chain consisting of SEQ ID NO: 100 or 83.

In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKASYRNYNYGD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 101). It will be understood that SEQ ID NO: 101 can also include the signal peptide of the invention (SEQ ID NO: 36) producing SEQ ID NO: 43 or contain a different signal peptide. In some embodiments, the antibody or antigen binding fragment thereof comprises a heavy chain consisting of SEQ ID NO: 101 or 43. In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKASYDPTNYYD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 102). It will be understood that SEQ ID NO: 102 can also include the signal peptide of the invention (SEQ ID NO: 36) producing SEQ ID NO: 44 or contain a different signal peptide. In some embodiments, the antibody or antigen binding fragment thereof comprises a heavy chain consisting of SEQ ID NO: 102 or 44. In some embodiments, the antibody or antigen binding fragment thereof comprises a heavy chain comprising QVQLVQSGGGLVQPGGSLRLSCAASGFTFSSYAMSWVRQAPGKGLEWVSAISGS GGSTYYADSVKGRFTISRDNSKNTLYLQMNSLRAEDTAVYYCAKASYRSTNYFD YWGQGTLVTVSSASTKGPSVFPLAPCSRSTSESTAALGCLVKDYFPEPVTVSWNSG ALTSGVHTFPAVLQSSGLYSLSSVVTVPSSSLGTKTYTCNVDHKPSNTKVDKRVES KYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 103). It will be understood that SEQ ID NO: 103 can also include the signal peptide of the invention (SEQ ID NO: 36) producing SEQ ID NO: 45 or contain a different signal peptide. In some embodiments, the antibody or antigen binding fragment thereof comprises a heavy chain consisting of SEQ ID NO: 103 or 45.

In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRX₂₂YYADSVEGRFTISRDNSKNTLYLQMNSLRAEDTAVYYCAKAPGDYTAYF DYWGQGTLVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKRVE SKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 108), wherein X₂₂ is any amino acid other than S and T. It will be understood that SEQ ID NO: 108 can also include the signal peptide of the invention (SEQ ID NO: 36) producing SEQ ID NO: 109 or contain a different signal peptide. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PNRX₂₃YYADSVEGRFTISRDNSKNTLYLQMNSLRAEDTAVYYCAKAPGDYTAYF DYWGQGTLVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKRVE SKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 110), wherein X₂₃ is any amino acid other than S, T and C. It will be understood that SEQ ID NO: 110 can also include the signal peptide of the invention (SEQ ID NO: 36) producing SEQ ID NO: 111 or contain a different signal peptide. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PX₂₄RTYYADSVEGRFTISRDNSKNTLYLQMNSLRAEDTAVYYCAKAPGDYTAYF DYWGQGTLVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKRVE SKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 115), wherein X₂₄ is any amino acid other than N. It will be understood that SEQ ID NO: 115 can also include the signal peptide of the invention (SEQ ID NO: 36) producing SEQ ID NO: 116 or contain a different signal peptide. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PX₂₅RTYYADSVEGRFTISRDNSKNTLYLQMNSLRAEDTAVYYCAKAPGDYTAYF DYWGQGTLVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKRVE SKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 117), wherein X₂₅ is any amino acid other than N and C. It will be understood that SEQ ID NO: 117 can also include the signal peptide of the invention (SEQ ID NO: 36) producing SEQ ID NO: 118 or contain a different signal peptide. In some embodiments, the antibody or antigen binding fragment thereof comprises a heavy chain comprising QVQLVQSGGGLVQPGGSLRLSCAASGFTFSSYAMSWVRQAPGKGLEWVSNIYSN PX₂₆RTYYADSVEGRFTISRDNSKNTLYLQMNSLRAEDTAVYYCAKAPGDYTAYF DYWGQGTLVTVSSASTKGPSVFPLAPCSRSTSESTAALGCLVKDYFPEPVTVSWNS GALTSGVHTFPAVLQSSGLYSLSSVVTVPSSSLGTKTYTCNVDHKPSNTKVDKRVE SKYGPPCPPCPAPEFLGGPSVFLFPPKPKDTLMISRTPEVTCVVVDVSQEDPEVQFN WYVDGVEVHNAKTKPREEQFNSTYRVVSVLTVLHQDWLNGKEYKCKVSNKGLP SSIEKTISKAKGQPREPQVYTLPPSQEEMTKNQVSLTCLVKGFYPSDIAVEWESNG QPENNYKTTPPVLDSDGSFFLYSRLTVDKSRWQEGNVFSCSVMHEALHNHYTQKS LSLSLGK (SEQ ID NO: 119), wherein X₂₆ is G, D, S, E, Q or N. It will be understood that SEQ ID NO: 119 can also include the signal peptide of the invention (SEQ ID NO: 36) producing SEQ ID NO: 120 or contain a different signal peptide. In some embodiments, the antibody or antigen binding fragment thereof comprises a heavy chain consisting of any one of SEQ ID NO: 108 to 111. In some embodiments, the antibody or antigen binding fragment thereof comprises a heavy chain consisting of any one of SEQ ID NO: 108 to 111. In some embodiments, the antibody or antigen binding fragment thereof comprises a heavy chain consisting of any one of SEQ ID NO: 115-120. In some embodiments, the antibody or antigen binding fragment thereof comprises a heavy chain consisting of any one of SEQ ID NO: 108-111 and 115-120.

In some embodiments, the antibody or antigen binding fragment comprises a heavy chain comprising a sequence selected from SEQ ID NO: 39-45, 52-53 and 75-83. In some embodiments, the antibody or antigen binding fragment comprises a heavy chain comprising a sequence selected from SEQ ID NO: 86-103. In some embodiments, the antibody or antigen binding fragment comprises a heavy chain comprising a sequence selected from SEQ ID NO: 9, 39-45, 52-53 and 75-83. In some embodiments, the antibody or antigen binding fragment comprises a heavy chain comprising a sequence selected from SEQ ID NO: 85-103. In some embodiments, the antibody or antigen binding fragment comprises a heavy chain consisting of a sequence selected from SEQ ID NO: 39-45, 52-53 and 75-83. In some embodiments, the antibody or antigen binding fragment comprises a heavy chain consisting of a sequence selected from SEQ ID NO: 86-103. In some embodiments, the antibody or antigen binding fragment comprises a heavy chain consisting of a sequence selected from SEQ ID NO: 9, 39-45, 52-53 and 75-83. In some embodiments, the antibody or antigen binding fragment comprises a heavy chain consisting of a sequence selected from SEQ ID NO: 85-103.

In some embodiments, the light chain comprises an N-terminal peptide comprising SEQ ID NO: 36. In some embodiments, the light chain comprises an N-terminal peptide consisting of SEQ ID NO: 36. In some embodiments, the light chain comprises an amino acid sequence of SEQ ID NO: 36. In some embodiments, the light chain comprises an amino acid sequence of SEQ ID NO: 36 N-terminal to any one of SEQ ID NO: 8 and SEQ ID NO: 35. In some embodiments, the N-terminal peptide is a signal peptide. In some embodiments, the light chain comprises a signal peptide. In some embodiments, the light chain is devoid of a signal peptide. In some embodiments, the light chain lacks a signal peptide. In some embodiments, the light chain comprises a signal peptide when first expressed in a cell and the signal peptide is cleaved and the secreted light chain lacks the signal peptide.

In some embodiments, the light chain comprises a C-terminal peptide comprising RTVAAPSVFIFPPSDEQLKSGTASVVCLLNNFYPREAKVQWKVDNALQSGNSQES VTEQDSKDSTYSLSSTLTLSKADYEKHKVYACEVTHQGLSSPVTKSFNRGEC (SEQ ID NO: 38). In some embodiments, the light chain comprises a C-terminal peptide consisting of SEQ ID NO: 38. In some embodiments, the C-terminal peptide is a light chain constant region. In some embodiments, the light chain comprises an amino acid sequence of SEQ ID NO: 38. In some embodiments, the light chain comprises an amino acid sequence of SEQ ID NO: 38 C-terminal to any one of SEQ ID NO: 8, and SEQ ID NO: 35. In some embodiments, the C-terminal peptide comprises a CL domain. In some embodiments, the CL is a kappa CL domain. In some embodiments, the CL is a lambda CL domain. It will be understood by a skilled artisan that the disulfide bridge that forms between the CH1 domain of the heavy chain and the CL domain of the light chain are sufficient for heavy chain-light chain dimerization and formation of a function antibody or antigen binding fragment. In some embodiments, the C-terminal peptide comprises a dimerization domain. In some embodiments, the dimerization domain is sufficient to induce dimerization between the heavy chain and the light chain.

In some embodiments, the antibody comprises a light chain comprising the sequence ELVLTQSPATLSLSPGERATLSCRASQSVSSYLAWYQQKPGQAPRLLIYGASSRAT GIPDRFSGSGSGTDFTLTISRLEPEDFAVYYCQQYGSSPPYTFGQGTKVEIKRTVAA PSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC (SEQ ID NO: 104). It will be understood that SEQ ID NO: 104 can also include the signal peptide of the invention (SEQ ID NO: 36) producing SEQ ID NO: 10 or contain a different signal peptide. In some embodiments, the antibody or antigen binding fragment thereof comprises a light chain comprising ELVLTQSPATLSLSPGERATLSCRASQSVSSYLAWYQQKPGQAPRLLIYGASSRAT GIPDRFSGSGSGTDFTLTISRLEPEDFAVYYCQQYGSYPPLTFGQGTKVEIKRTVAA PSVFIFPPSDEQLKSGTASVVCLLNNFYPREAKVQWKVDNALQSGNSQESVTEQDS KDSTYSLSSTLTLSKADYEKHKVYACEVTHQGLSSPVTKSFNRGEC (SEQ ID NO: 105). It will be understood that SEQ ID NO: 105 can also include the signal peptide of the invention (SEQ ID NO: 36) producing SEQ ID NO: 46 or contain a different signal peptide. In some embodiments, the antibody or antigen binding fragment thereof comprises a light chain consisting of SEQ ID NO: 104 or 10. In some embodiments, the antibody or antigen binding fragment thereof comprises a light chain consisting of SEQ ID NO: 105 or 46. In some embodiments, the antibody or antigen binding fragment comprises a light chain comprising a sequence selected from SEQ ID NO: 10 and SEQ ID NO: 46. In some embodiments, the antibody or antigen binding fragment comprises a light chain comprising a sequence selected from SEQ ID NO: 104 and SEQ ID NO: 105. In some embodiments, the antibody or antigen binding fragment comprises a light chain consisting of a sequence selected from SEQ ID NO: 10 and SEQ ID NO: 46. In some embodiments, the antibody or antigen binding fragment comprises a light chain consisting of a sequence selected from SEQ ID NO: 104 and SEQ ID NO: 105.

In some embodiments, the heavy chain and the light chain are joined by a linker. In some embodiments, the linker is an amino acid linker. In some embodiments, the linker is at most 1, 2, 3, 4, 5, 6, 7, 8, 9, 10, 12, 14, 15, 16, 18, 20, 22, 24 or 25 amino acids long. Each possibility represents a separate embodiment of the invention. In some embodiments, the linker is at least 1, 2, 3, 4, 5, 6, 7, 8, 9, 10, 12, 14, 15, 16, 18, 20, 22, 24 or 25 amino acids long. Each possibility represents a separate embodiment of the invention. In some embodiments, the linker is between 1-25, 5-25, 10-25, 15-25, 20-25, 1-20, 5-20, 10-20, 15-20, 1-15, 5-15, 10-15, 1-10, 5-10 or 1-5 amino acids long. Each possibility represents a separate embodiment of the invention.

In some embodiments, the antibody or antigen binding fragment thereof is selected from those provided in Table 1.

TABLE 1
Antibodies of the invention

| | | Heavy | Heavy | | Light | Light |
|---|---|---|---|---|---|---|
| | Heavy | chain | com- | Light | chian | com- |
| | variable | no SP | plete | variable | no | plete |
| | SEQ ID | SEQ | SEQ | SEQ ID | SP SEQ | SEQ ID |
| Name | NO: | ID NO: | ID NO: | NO: | ID NO: | NO: |
| Parental | 7 | 85 | 9 | 8 | 104 | 10 |
| H1 | 32 | 101 | 43 | 8 | 104 | 10 |
| H3 | 33 | 102 | 44 | 8 | 104 | 10 |
| H12 | 34 | 103 | 45 | 8 | 104 | 10 |
| H4 | 30 | 88 | 41 | 8 | 104 | 10 |
| H4-E64K | 50 | 90 | 52 | 8 | 104 | 10 |
| H4-N55D | 66 | 92 | 75 | 8 | 104 | 10 |
| H4-N55E | 67 | 93 | 76 | 8 | 104 | 10 |
| H4-N55G | 68 | 94 | 77 | 8 | 104 | 10 |
| H4-N55Q | 69 | 95 | 78 | 8 | 104 | 10 |
| H4-N55S | 70 | 96 | 79 | 8 | 104 | 10 |
| H4-T57A | 71 | 97 | 80 | 8 | 104 | 10 |
| H4-T57E | 72 | 98 | 81 | 8 | 104 | 10 |
| H4-T57G | 73 | 99 | 82 | 8 | 104 | 10 |
| H4-T57N | 74 | 100 | 83 | 8 | 104 | 10 |
| H5 | 31 | 89 | 42 | 8 | 104 | 10 |
| H6 | 28 | 86 | 39 | 8 | 104 | 10 |
| H8 | 29 | 87 | 40 | 8 | 104 | 10 |
| Par-K64E | 51 | 91 | 53 | 8 | 104 | 10 |
| L18 | 7 | 85 | 9 | 35 | 105 | 46 |
| L18-H4 | 30 | 88 | 41 | 35 | 105 | 46 |
| L18-H5 | 31 | 89 | 42 | 35 | 105 | 46 |

In some embodiments, the antibody comprises a heavy chain variable region comprising or consisting of SEQ ID NO: 32 and a light chain variable region comprising or consisting of SEQ ID NO: 8. In some embodiments, the antibody comprises a heavy chain comprising or consisting of SEQ ID NO: 101 and a light chain comprising or consisting of SEQ ID NO: 104. In some embodiments, the antibody comprises a heavy chain comprising or consisting of SEQ ID NO: 43 and a light chain comprising or consisting of SEQ ID NO: 10.

In some embodiments, the antibody comprises a heavy chain variable region comprising or consisting of SEQ ID NO: 33 and a light chain variable region comprising or consisting of SEQ ID NO: 8. In some embodiments, the antibody comprises a heavy chain comprising or consisting of SEQ ID NO: 102 and a light chain comprising or consisting of SEQ ID NO: 104. In some embodiments, the antibody comprises a heavy chain comprising or consisting of SEQ ID NO: 44 and a light chain comprising or consisting of SEQ ID NO: 10.

In some embodiments, the antibody comprises a heavy chain variable region comprising or consisting of SEQ ID NO: 34 and a light chain variable region comprising or consisting of SEQ ID NO: 8. In some embodiments, the antibody comprises a heavy chain comprising or consisting of SEQ ID NO: 103 and a light chain comprising or consisting of SEQ ID NO: 104. In some embodiments, the antibody comprises a heavy chain comprising or consisting of SEQ ID NO: 45 and a light chain comprising or consisting of SEQ ID NO: 10.

In some embodiments, the antibody comprises a heavy chain variable region comprising or consisting of SEQ ID NO: 30 and a light chain variable region comprising or consisting of SEQ ID NO: 8. In some embodiments, the antibody comprises a heavy chain comprising or consisting of SEQ ID NO: 88 and a light chain comprising or consisting of SEQ ID NO: 104. In some embodiments, the antibody comprises a heavy chain comprising or consisting of SEQ ID NO: 41 and a light chain comprising or consisting of SEQ ID NO: 10.

In some embodiments, the antibody comprises a heavy chain variable region comprising or consisting of SEQ ID NO: 50 and a light chain variable region comprising or consisting of SEQ ID NO: 8. In some embodiments, the antibody comprises a heavy chain comprising or consisting of SEQ ID NO: 90 and a light chain comprising or consisting of SEQ ID NO: 104. In some embodiments, the antibody comprises a heavy chain comprising or consisting of SEQ ID NO: 52 and a light chain comprising or consisting of SEQ ID NO: 10.

In some embodiments, the antibody comprises a heavy chain variable region comprising or consisting of SEQ ID NO: 66 and a light chain variable region comprising or consisting of SEQ ID NO: 8. In some embodiments, the antibody comprises a heavy chain comprising or consisting of SEQ ID NO: 92 and a light chain comprising or consisting of SEQ ID NO: 104. In some embodiments, the antibody comprises a heavy chain comprising or consisting of SEQ ID NO: 75 and a light chain comprising or consisting of SEQ ID NO: 10.

In some embodiments, the antibody comprises a heavy chain variable region comprising or consisting of SEQ ID NO: 67 and a light chain variable region comprising or consisting of SEQ ID NO: 8. In some embodiments, the antibody comprises a heavy chain comprising or consisting of SEQ ID NO: 93 and a light chain comprising or consisting of SEQ ID NO: 104. In some embodiments, the antibody comprises a heavy chain comprising or consisting of SEQ ID NO: 76 and a light chain comprising or consisting of SEQ ID NO: 10.

In some embodiments, the antibody comprises a heavy chain variable region comprising or consisting of SEQ ID NO: 68 and a light chain variable region comprising or consisting of SEQ ID NO: 8. In some embodiments, the antibody comprises a heavy chain comprising or consisting of SEQ ID NO: 94 and a light chain comprising or consisting of SEQ ID NO: 104. In some embodiments, the antibody comprises a heavy chain comprising or consisting of SEQ ID NO: 77 and a light chain comprising or consisting of SEQ ID NO: 10.

In some embodiments, the antibody comprises a heavy chain variable region comprising or consisting of SEQ ID NO: 69 and a light chain variable region comprising or consisting of SEQ ID NO: 8. In some embodiments, the antibody comprises a heavy chain comprising or consisting of SEQ ID NO: 95 and a light chain comprising or consisting of SEQ ID NO: 104. In some embodiments, the antibody comprises a heavy chain comprising or consisting of SEQ ID NO: 78 and a light chain comprising or consisting of SEQ ID NO: 10.

In some embodiments, the antibody comprises a heavy chain variable region comprising or consisting of SEQ ID NO: 70 and a light chain variable region comprising or consisting of SEQ ID NO: 8. In some embodiments, the antibody comprises a heavy chain comprising or consisting of SEQ ID NO: 96 and a light chain comprising or consisting of SEQ ID NO: 104. In some embodiments, the antibody comprises a heavy chain comprising or consisting of SEQ ID NO: 79 and a light chain comprising or consisting of SEQ ID NO: 10.

In some embodiments, the antibody comprises a heavy chain variable region comprising or consisting of SEQ ID NO: 71 and a light chain variable region comprising or consisting of SEQ ID NO: 8. In some embodiments, the antibody comprises a heavy chain comprising or consisting of SEQ ID NO: 97 and a light chain comprising or consisting of SEQ ID NO: 104. In some embodiments, the antibody comprises a heavy chain comprising or consisting of SEQ ID NO: 80 and a light chain comprising or consisting of SEQ ID NO: 10.

In some embodiments, the antibody comprises a heavy chain variable region comprising or consisting of SEQ ID NO: 72 and a light chain variable region comprising or consisting of SEQ ID NO: 8. In some embodiments, the antibody comprises a heavy chain comprising or consisting of SEQ ID NO: 98 and a light chain comprising or consisting of SEQ ID NO: 104. In some embodiments, the antibody comprises a heavy chain comprising or consisting of SEQ ID NO: 81 and a light chain comprising or consisting of SEQ ID NO: 10.

In some embodiments, the antibody comprises a heavy chain variable region comprising or consisting of SEQ ID NO: 73 and a light chain variable region comprising or consisting of SEQ ID NO: 8. In some embodiments, the antibody comprises a heavy chain comprising or consisting of SEQ ID NO: 99 and a light chain comprising or consisting of SEQ ID NO: 104. In some embodiments, the antibody comprises a heavy chain comprising or consisting of SEQ ID NO: 82 and a light chain comprising or consisting of SEQ ID NO: 10.

In some embodiments, the antibody comprises a heavy chain variable region comprising or consisting of SEQ ID NO: 74 and a light chain variable region comprising or consisting of SEQ ID NO: 8. In some embodiments, the antibody comprises a heavy chain comprising or consisting of SEQ ID NO: 100 and a light chain comprising or consisting of SEQ ID NO: 104. In some embodiments, the antibody comprises a heavy chain comprising or consisting of SEQ ID NO: 83 and a light chain comprising or consisting of SEQ ID NO: 10.

In some embodiments, the antibody comprises a heavy chain variable region comprising or consisting of SEQ ID NO: 31 and a light chain variable region comprising or consisting of SEQ ID NO: 8. In some embodiments, the antibody comprises a heavy chain comprising or consisting of SEQ ID NO: 89 and a light chain comprising or consisting of SEQ ID NO: 104. In some embodiments, the antibody comprises a heavy chain comprising or consisting of SEQ ID NO: 42 and a light chain comprising or consisting of SEQ ID NO: 10.

In some embodiments, the antibody comprises a heavy chain variable region comprising or consisting of SEQ ID NO: 28 and a light chain variable region comprising or consisting of SEQ ID NO: 8. In some embodiments, the antibody comprises a heavy chain comprising or consisting of SEQ ID NO: 86 and a light chain comprising or consisting of SEQ ID NO: 104. In some embodiments, the antibody comprises a heavy chain comprising or consisting of SEQ ID NO: 39 and a light chain comprising or consisting of SEQ ID NO: 10.

In some embodiments, the antibody comprises a heavy chain variable region comprising or consisting of SEQ ID NO: 29 and a light chain variable region comprising or consisting of SEQ ID NO: 8. In some embodiments, the antibody comprises a heavy chain comprising or consisting of SEQ ID NO: 87 and a light chain comprising or consisting of SEQ ID NO: 104. In some embodiments, the antibody comprises a heavy chain comprising or consisting of SEQ ID NO: 40 and a light chain comprising or consisting of SEQ ID NO: 10.

In some embodiments, the antibody comprises a heavy chain variable region comprising or consisting of SEQ ID NO: 51 and a light chain variable region comprising or consisting of SEQ ID NO: 8. In some embodiments, the antibody comprises a heavy chain comprising or consisting of SEQ ID NO: 91 and a light chain comprising or consisting of SEQ ID NO: 104. In some embodiments, the antibody comprises a heavy chain comprising or consisting of SEQ ID NO: 53 and a light chain comprising or consisting of SEQ ID NO: 10.

In some embodiments, the antibody comprises a heavy chain variable region comprising or consisting of SEQ ID NO: 7 and a light chain variable region comprising or consisting of SEQ ID NO: 35. In some embodiments, the antibody comprises a heavy chain comprising or consisting of SEQ ID NO: 85 and a light chain comprising or consisting of SEQ ID NO: 105. In some embodiments, the antibody comprises a heavy chain comprising or consisting of SEQ ID NO: 9 and a light chain comprising or consisting of SEQ ID NO: 46.

In some embodiments, the antibody comprises a heavy chain variable region comprising or consisting of SEQ ID NO: 30 and a light chain variable region comprising or consisting of SEQ ID NO: 35. In some embodiments, the antibody comprises a heavy chain comprising or consisting of SEQ ID NO: 88 and a light chain comprising or consisting of SEQ ID NO: 105. In some embodiments, the antibody comprises a heavy chain comprising or consisting of SEQ ID NO: 41 and a light chain comprising or consisting of SEQ ID NO: 46.

In some embodiments, the antibody comprises a heavy chain variable region comprising or consisting of SEQ ID NO: 31 and a light chain variable region comprising or consisting of SEQ ID NO: 35. In some embodiments, the antibody comprises a heavy chain comprising or consisting of SEQ ID NO: 89 and a light chain comprising or consisting of SEQ ID NO: 105. In some embodiments, the antibody comprises a heavy chain comprising or consisting of SEQ ID NO: 42 and a light chain comprising or consisting of SEQ ID NO: 46.

By another aspect, there is provided an antibody or antigen binding fragment comprising at least 70% amino acid identity to an antibody or antigen binding fragment provided in Table 1 and comprising the same CDRs as that antibody.

It will be understood by a skilled artisan that the CDRs define the binding specificity of the antibody/binding fragment and therefore, so long as the CDRs are retained the sequence around the CDRs can be modified/altered. In some embodiments, at least 70% is at least 75%, 80%, 85%, 90%, 92%, 95%, 97%, 99% or 100%. Each possibility represents a separate embodiment of the invention. In some embodiments, at least 70% is at least 80%. In some embodiments, at least 70% is at least 90%.

Engineered antibodies of the invention include those in which modifications have been made to framework residues within VH and/or VL, e.g., to improve the properties of the antibody. Typically, such framework modifications are made to decrease the immunogenicity of the antibody. For example, one approach is to “backmutate” one or more framework residues to the corresponding germline sequence. More specifically, an antibody that has undergone somatic mutation can contain framework residues that differ from the germline sequence from which the antibody is derived. Such residues can be identified by comparing the antibody framework sequences to the germline sequences from which the antibody is derived.

For example, in the 4C7 VH region a framework amino acid at position 25, 68 and 82a all differ from the germline and can be backmutated to the germline by substitutions H25S, S68T and T82aT respectively.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043.

In addition, or alternative to modifications made within the framework or CDR regions, antibodies of the invention can be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention can be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In a preferred embodiment, the antibody is an IgG4 isotype antibody comprising a Serine to Proline mutation at a position corresponding to position 228 (S228P; EU index) in the hinge region of the heavy chain constant region. This mutation has been reported to abolish the heterogeneity of inter-heavy chain disulfide bridges in the hinge region (Angal et al. supra; position 241 is based on the Kabat numbering system).

In one embodiment, the hinge region of CH1 is modified such that the number of cysteine residues in the hinge region is altered, e.g., increased or decreased. This approach is described further in U.S. Pat. No. 5,677,425. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745.

In another embodiment, the antibody is modified to increase its biological half-life. Various approaches are possible. For example, one or more of the following mutations can be introduced: T252L, T254S, T256F, as described in U.S. Pat. No. 6,277,375. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551.

In another example, one or more amino acid residues within amino acid positions 231 and 239 are altered to thereby alter the ability of the antibody to fix complement. This approach is described further in PCT Publication WO 94/29351.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072. Moreover, the binding sites on human IgG1 for FcγR1, FcγRII, FcγRIII and FcRn have been mapped and variants with improved binding have been described (see Shields et al. (2001) J. Biol. Chem. 276:6591-6604). Specific mutations at positions 256, 290, 298, 333, 334 and 339 were shown to improve binding to FcγRIII Additionally, the following combination mutants were shown to improve FcγRIII binding: T256A/S298A, S298A/E333A, S298A/K224A and S298A/E333A/K334A.

In still another embodiment, the glycosylation of an antibody is modified. For example, an aglycosylated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. See, e.g., U.S. Pat. Nos. 5,714,350 and 6,350,861.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies.

Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (α (1,6)-fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see U.S. Patent Publication No. 20040110704 and Yamane-Ohnuki et al. (2004) Biotechnol Bioeng 87:614-22). As another example, EP 1,176,195 describes a cell line with a functionally disrupted FUT8 gene, which encodes a fucosyl transferase, such that antibodies expressed in such a cell line exhibit hypofucosylation by reducing or eliminating the α-1,6 bond-related enzyme. EP 1,176,195 also describes cell lines which have a low enzyme activity for adding fucose to the N-acetylglucosamine that binds to the Fc region of the antibody or does not have the enzyme activity, for example the rat myeloma cell line YB2/0 (ATCC® CRL 1662). PCT Publication WO 03/035835 describes a variant CHO cell line, Lec13 cells, with reduced ability to attach fucose to Asn(297)-linked carbohydrates, also resulting in hypofucosylation of antibodies expressed in that host cell (see also Shields et al. (2002) J. Biol. Chem. 277:26733-26740). Antibodies with a modified glycosylation profile can also be produced in chicken eggs, as described in PCT Publication WO 06/089231. Alternatively, antibodies with a modified glycosylation profile can be produced in plant cells, such as Lemna (U.S. Pat. No. 7,632,983). Methods for production of antibodies in a plant system are disclosed in the U.S. Pat. Nos. 6,998,267 and 7,388,081. PCT Publication WO 99/54342 describes cell lines engineered to express glycoprotein-modifying glycosyl transferases (e.g., β(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17:176-180). Alternatively, the fucose residues of the antibody can be cleaved off using a fucosidase enzyme; e.g., the fucosidase α-L-fucosidase removes fucosyl residues from antibodies (Tarentino et al. (1975) Biochem. 14:5516-23).

Another modification of the antibodies herein that is contemplated by this disclosure is pegylation. An antibody can be pegylated to, for example, increase the biological (e.g., serum) half-life of the antibody. To pegylate an antibody, the antibody, or fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See, e.g., EP 0154316 and EP 0401384.

By another aspect, there is provided a pharmaceutical composition comprising an antibody or antigen binding fragment thereof of the invention.

In some embodiments, pharmaceutical composition further comprises a pharmaceutically acceptable carrier, excipient or adjuvant. In some embodiments, pharmaceutical composition a therapeutically effective amount of the antibody or antigen binding fragment thereof of the invention.

As used herein, the term “carrier,” “excipient,” or “adjuvant” refers to any component of a pharmaceutical composition that is not the active antibody or antigen binding fragment thereof. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering antibodies or antigen binding fragments thereof such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting antibodies or antigen binding fragments thereof and lubricants such as sodium lauryl sulfate, as well as coloring antibodies or antigen binding fragments thereof, flavoring antibodies or antigen binding fragments thereof, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.10% to about 99.99999% by weight of the pharmaceutical compositions presented herein.

The term “therapeutically effective amount” refers to an amount of a drug effective to treat a disease or disorder in a mammal. In some embodiments, a therapeutically effective amount is an amount effective, at dosages and for periods of time necessary, to achieve the desired therapeutic or prophylactic result. The exact dosage form and regimen would be determined by the physician according to the patient's condition. In some embodiments, the pharmaceutical composition comprises a therapeutically effective amount of the antibody or antigen binding fragment of the invention. In some embodiments, the pharmaceutical composition is a therapeutic composition.

In some embodiments, the antibody or antigen binding fragment thereof is for use in treating a disease or condition. In some embodiments, the pharmaceutical composition is for use in treating a disease or condition. In some embodiments, the disease or condition is characterized by cells expressing HVEM. In some embodiments, the cell are disease cells characterized by expressing HVEM. In some embodiments, the cells are characterized by overexpressing HVEM. In some embodiments, overexpression is as compared to a healthy cell. In some embodiments, a healthy cell is a non-diseased cell. In some embodiments, overexpression is an increased expression. In some embodiments, the disease or condition is an HVEM positive disease or condition. In some embodiments, the disease or condition is a BTLA positive disease or condition.

In some embodiments, the disease or condition is an HVEM positive cancer. In some embodiments, the disease or condition is a cancer comprising BTLA positive resident cells. In some embodiments, the disease or condition is an HVEM positive precancerous lesion. In some embodiments, the cancer is melanoma. In some embodiments, the cancer is renal cell carcinoma. In some embodiments, the cancer is selected from melanoma, renal cancer, cervical cancer, prostate cancer, pancreatic cancer, lung cancer, ovarian cancer, colon cancer, breast cancer, hepatocellular cancer, cholangiocarcinoma, thymoma and head and neck cancer. In some embodiments, the renal cancer is renal cell carcinoma. A skilled artisan will appreciate that any cancer that expresses HVEM may be a therapeutic target. In some embodiments, the disease is an infectious disease. In some embodiments, the cells of the infection comprise HVEM expression. In some embodiments, an infected cell comprises HVEM expression. In some embodiments, the infectious disease is a bacterium, and a bacterial cell comprises HVEM expression. In some embodiments, the infectious disease is a fungal infection, and a fungal cell comprises HVEM expression. In some embodiments, the infectious disease is a virus, and a virally infected subject cell comprises HVEM expression. In some embodiments, the virus is Herpes virus. In some embodiments, HVEM expression is HVEM overexpression. In some embodiments, HVEM expression is increased HVEM expression.

By another aspect, there is provided a method of treating an HVEM positive disease or condition in a subject in need thereof, the method comprising administering to the subject an antibody or antigen binding fragment thereof of the invention, thereby treating the subject.

By another aspect, there is provided a method of treating an HVEM positive disease or condition in a subject in need thereof, the method comprising administering to the subject a pharmaceutical composition of the invention, thereby treating the subject.

In some embodiments, the method further comprises not inhibiting HVEM-TNFSF14 interaction. In some embodiments, the method further comprises not substantially inhibiting HVEM-TNFSF14 interaction. In some embodiments, the method further comprises not fully inhibiting HVEM-TNFSF14 interaction.

In some embodiments, the method further comprises immune checkpoint inhibition of a non-HVEM checkpoint protein. In some embodiments, the non-HVEM checkpoint protein is the PD-1. In some embodiments, the method further comprises immune checkpoint blockade of a non-HVEM checkpoint protein. In some embodiments, the method further comprises inhibiting interaction between PD-1 and one of its ligands. In some embodiments, a PD-1 ligand is selected from PD-L1 and PD-L2. In some embodiments, the method further comprises inhibiting PD-1, PD-L1 or PD-L2. In some embodiments, the method further comprises inhibiting PD-1 to PD-L1 interaction. In some embodiments, the method further comprises inhibiting PD-1 to PD-L2 interaction. In some embodiments, the method further comprises inhibiting PD-1 to PD-L1 or PD-L2 interaction. In some embodiments, inhibiting interaction comprises administering an antibody or antigen binding fragment thereof that inhibits PD-1 to PD-L1 interaction or PD-1 to PD-L2 interaction. In some embodiments, inhibiting interaction comprises administering an antibody or antigen binding fragment thereof that inhibits interaction between PD-1 and at least one of its ligands. In some embodiments, at least one ligand is two ligands. In some embodiments, inhibiting interaction comprises PD-1/PD-L1 blockade. In some embodiments, inhibiting interaction comprises PD-1/PD-L1 or PD-L2 blockade. In some embodiments, inhibiting interaction comprises anti-PD-1/PD-L1 therapy. In some embodiments, inhibiting interaction comprises anti-PD-1/PD-L1 or PD-L2 therapy. In some embodiments, therapy is immunotherapy. In some embodiments, an antibody or antigen binding fragment thereof that inhibits interaction is an anti-PD-1 or anti-PD-L1 antibody. In some embodiments, an antibody or antigen binding fragment thereof that inhibits interaction is an anti-PD-1, anti-PD-L1 or anti-PD-L2 antibody. In some embodiments, an antibody or antigen binding fragment thereof that inhibits interaction is a PD-1/PD-L1 inhibitor. In some embodiments, an antibody or antigen binding fragment thereof that inhibits interaction is a PD-1/PD-L1/PD-L2 inhibitor. In some embodiments, the antibody is a blocking antibody. PD-L1/L2 and PD-1 therapies are well known in the art and include but are not limited to nivolumab (Opdivo), pembrolizumab (Keytruda), atezolizumab, avelumab, durvalumab, cemiplimab (Libtayo), pidilizumab, AMP-224, AMP-514 and PDR001.

In some embodiments, the method of treatment further comprises administering another therapeutic against the HVEM-expressing cell. In some embodiments, the other therapeutic is an anti-cancer therapeutic. In some embodiments, the therapeutic is a non-autologous immune cell. In some embodiments, the other therapeutic is an autologous immune cell. In some embodiments, the immune cell is a CAR expression immune cell. In some embodiments, the CAR is a CAR-T. In some embodiments, the CAR is a CAR-NK.

In some embodiments, the autologous immune cell is TIL therapy. In some embodiments, the non-autologous immune cell is an adoptive cell therapy. In some embodiments, the autologous immune cell is an adoptive cell therapy. In some embodiments, the adoptive cell is a CAR cell. In some embodiments, the adoptive cell is a TIL.

In some embodiments, the composition of the invention and the another therapeutic are administering concomitantly. In some embodiments, the composition of the invention is administered before, after or at the same time as the another therapeutic.

In some embodiments, the subject is a human. In some embodiments, the subject is a subject suffering from a HVEM positive disease or condition. In some embodiments, the subject is suffering from a disease characterized by HVEM positive cells. In some embodiments, the subject is suffering from cancer. In some embodiments, the cancer is HVEM positive cancer. In some embodiments, the subject is naïve to therapy. In some embodiments, the subject is naïve to anti-HVEM therapy. In some embodiments, the subject is naïve to immunotherapy.

As used herein, the terms “administering,” “administration,” and like terms refer to any method which, in sound medical practice, delivers a composition containing an active agent to a subject in such a manner as to provide a therapeutic effect. One aspect of the present subject matter provides for intravenous administration of a therapeutically effective amount of a composition of the present subject matter to a patient in need thereof. Other suitable routes of administration can include parenteral, subcutaneous, oral, intramuscular, intrathecal, or intraperitoneal.

The dosage administered will be dependent upon the age, health, and weight of the recipient, kind of concurrent treatment, if any, frequency of treatment, and the nature of the effect desired.

By another aspect, there is provided a method of determining suitability of a subject to be treated by a method of the invention, the method comprising obtaining a sample from the subject and determining HVEM levels in the sample, wherein positive expression of HVEM indicates the subject is suitable for a method of treatment of the invention.

By another aspect, there is provide a method of detecting HVEM in a sample, the method comprising contacting the sample with an antibody or antigen binding fragment thereof of the invention, or an antibody or antigen binding fragment thereof of the invention, thereby detecting HVEM.

In some embodiments, determining HVEM levels comprises detecting HVEM. In some embodiments, the detecting comprises quantitating the amount of HVEM. In some embodiments, the contacting is under conditions suitable for binding of the antibody or antigen binding fragment thereof or antibody or antigen binding fragment thereof to bind to HVEM. In some embodiments, the conditions are suitable for specific binding to HVEM. In some embodiments, binding is hybridizing. Conditions for antibody/antibody or antigen binding fragment thereof binding will depend on the sample. A skilled artisan will appreciate the conditions needed for binding to a tissue sample (dependent on the method/type of fixation) are different than those of binding in a liquid solution, such as a whole cell lysate. Such binding conditions are well known in the art and a skilled artisan can select the proper conditions for a given sample.

In some embodiments, the sample is a tissue sample. In some embodiments, the sample is a paraffin embedded sample. In some embodiments, the sample is a perfused sample. In some embodiments, the sample is from a subject. In some embodiments, the sample is a healthy sample. In some embodiments, the sample is a diseased sample. In some embodiments, the sample is a diagnostic sample.

In some embodiments, the method further comprises detecting the antibody or antigen binding fragment thereof of the invention, or an antibody or antigen binding fragment thereof of the invention. In some embodiments, the detection is immunohistochemical detection. In some embodiments, the detection is immunofluorescent detection. In some embodiments, the detection is FACS detection. In some embodiments, the detection further comprises contacting the sample with a secondary antibody that recognized the antibody or antigen binding fragment thereof or antibody of the invention. In some embodiments, the detection comprises detecting the secondary antibody. In some embodiments, the detection comprises microscopy.

In some embodiments, the subject suffers from a disease or condition. In some embodiments, the subject is suspected to suffer from a disease or condition. In some embodiments, the subject is at risk of developing a disease or condition. In some embodiments, the subject suffers from a disease or condition which may comprise increased HVEM expression. In some embodiments, the subject suffers from a disease or condition which may be characterized by increased HVEM expression. In some embodiments, the subject suffers from cancer. In some embodiments, the cancer is a cancer that did not respond to first line therapy. In some embodiments, the cancer is a cancer relapse. In some embodiments, the cancer is a cancer that did not respond to PD-1/PD-L1 therapy.

In some embodiments, the sample is a disease sample. In some embodiments, the sample is a biological fluid. In some embodiments, the biological fluid is selected from blood, serum, plasma, urine, feces, bile, semen, tumor fluid, and cerebral spinal fluid. In some embodiments, the sample is a blood sample. In some embodiments, the sample is a cell sample. In some embodiments, the sample comprises cells. In some embodiments, the sample comprises disease cells. In some embodiments, the sample is a tumor sample. In some embodiments, the sample is a biopsy.

In some embodiments, positive expression of HVEM comprises HVEM expression.

In some embodiments, positive expression of HVEM comprises elevated HVEM levels. In some embodiments, positive expression of HVEM comprises increased HVEM levels. In some embodiments, positive expression of HVEM comprises overexpression of HVEM. In some embodiments, positive expression of HVEM is as compared to healthy sample. In some embodiments, the healthy sample is from healthy tissue and/or cells adjacent to the disease tissue and/or cells. In some embodiments, a healthy sample comprises non-infected cells. In some embodiments, a healthy sample is for a healthy donor. In some embodiments, positive expression of HVEM is as compared to a predetermined threshold.

By another aspect, there is provided a kit comprising a pharmaceutical composition of the invention, or an antibody or antigen binding fragment thereof of the invention.

In some embodiments, the kit further comprises a PD-1 based therapy. In some embodiments, the kit further comprises a PD-L1 based therapy. In some embodiments, the therapy is an immunotherapy. In some embodiments, the therapy is an anti-PD-1 therapy. In some embodiments, the therapy is an anti-PD-L1 therapy. In some embodiments, therapy is a PD-1/PD-L1 blockade therapy. In some embodiments, the therapy is a blocking antibody.

In some embodiments, the therapy is an anti-PD-1 antibody. In some embodiments, the therapy is an anti-PD-L1 antibody. In some embodiments, the antibody is a blocking antibody.

In some embodiments, the kit further comprises a label stating the pharmaceutical composition of the invention is for use with a PD-1 and/or PD-L1 based therapy. In some embodiments, the kit further comprises a label stating the antibody or antigen binding fragment thereof of the invention is for use with a PD-1 and/or PD-L1 based therapy.

In some embodiments, the kit further comprises a secondary detection molecule. In some embodiments, the detection molecule is for detection an antibody or antigen binding fragment thereof of the invention. In some embodiments, the secondary detection molecule is an antibody that binds to an antibody or antigen binding fragment thereof of the invention. In some embodiments, the detection molecule comprises a detectable moiety. Examples of detectable moieties include, but are not limited to a fluorescent moiety, a tag, a radiolabel, a dye and a chemiluminescent moiety. In some embodiments, the detectable moiety is a fluorescent moiety. Examples of fluorescent moieties include, but are not limited to GFP, RFP, YFP, APC, CY5, CY7, and Pacific Blue. Secondary detection molecules are well known in the art and any such molecule that will detect an antibody or antigen binding fragment thereof of the invention may be used.

By another aspect, there is provided a nucleic acid molecule encoding an antibody or antigen binding fragment of the invention.

By another aspect, there is provided a nucleic acid molecule encoding a heavy chain of an antibody or antigen binding fragment of the invention and a nucleic acid molecule encoding a light chain of an antibody or antigen binding fragment of the invention.

In some embodiments, the nucleic acid molecule is configured for expression. In some embodiments, expression is expression in a cell. In some embodiments, expression is expression in an in vitro expression system. The term “expression” as used herein refers to the biosynthesis of a coding region, including the transcription and/or translation of the coding region. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, the coding region encodes the antibody or antigen binding fragment of the invention. In some embodiments, the coding region encodes the heavy chain. In some embodiments, the coding region encodes the light chain.

Expressing of a coding region within a cell is well known to one skilled in the art. It can be carried out by, among many methods, transfection, viral infection, or direct alteration of the cell's genome. In some embodiments, the coding region is in an expression vector such as plasmid or viral vector. In some embodiments, the nucleic acid molecule is a vector.

In some embodiments, the vector is an expression vector.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the coding region is operably linked to a regulatory element.

In some embodiments, the coding region is operably linked to a promoter. In some embodiments, the regulatory element is a protein. In some embodiments, the regulatory element is an enhancer. The term “operably linked” is intended to mean that the nucleotide sequence of interest is linked to the regulatory element or elements in a manner that allows for expression of the nucleotide sequence (e.g., in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, C T (1994); Mishell and Shiigi (eds), “Strategies for Protein Purification and Characterization—A Laboratory Course Manual” CSHL Press (1996); all of which are incorporated by reference. Other general references are provided throughout this document.

### Materials and Methods

Affinity maturation: To improve binding affinity, the parental antibody was affinity matured using phage display. Heavy and/or light chain CDRs of variable domain regions were semi-randomized using oligonucleotide mutagenesis. Selections were performed using separate heavy and light chain phage libraries and decreasing concentrations of biotinylated His-tagged HVEM target to select the highest affinity binders from each library. Following four rounds of selections individual clones were screened as scFv supematants in a binding ELISA to identify lead candidates with signals greater than the parental scFv expressed in the same assay. Identified hits were converted into IgG and cloned into mammalian expression vectors. Plasmids encoding the affinity improved variants paired with the appropriate parental heavy or light chain, as well as the parental antibody, were transiently transfected small scale into HEK EBNA adherent cells (LGC Standards, Teddington, UK). At five days post-transfection, the supernatants were harvested, quantified by Octet (serial no. FB-30203) and analysed by Biacore single cycle kinetics.

Single Cycle Kinetics: To assess the binding of the affinity matured IgGs, single cycle kinetic analysis was performed on supernatants using a Biacore T200 (serial no. 1909913). Kinetic experiments were performed at 25° C. running Biacore T200 Control software V2.0.1 and Evaluation software V3.0 (Cytiva, Marlborough, USA). Antibodies were diluted in HBS-P+(Cytiva, Marlborough, USA) containing 1 mg/ml BSA to a final concentration of 1.0 μg/ml. Antibodies were loaded onto Fc2, Fc3 and Fc4 of the Protein A capture Sensor chip (Cytiva, Marlborough, USA). IgGs were captured at a flow rate of 10 μl/min to give an immobilisation level (RL) of ˜50 RU. The surface was then allowed to stabilize. Single cycle kinetics data was obtained using His-tagged HVEM (Acro Biosystems, Newark, USA) as the analyte at a flow rate of 40 μl/min. A two-fold dilution range from 6.125 nM to 100 nM His-tagged antigen without regeneration between each concentration was used.

Multi-Cycle Kinetics: Multicycle kinetic analysis was performed on purified antibodies. Kinetic experiments were performed at 25° C. on a Biacore 8K. HBS-P+0.1% BSA (Cytiva, Marlborough, USA) was used as running buffer. Purified antibodies were diluted to 1.0 μg/mL in running buffer and at the start of each cycle, loaded onto Fc2, Fc3 and Fc4 of a Protein A sensor chip (Cytiva, Marlborough, USA). Antibodies were captured at a flow rate of 10 μl/min to give an immobilisation level (RL) of ˜ 100 RU. The surface was then allowed to stabilise. Multi-cycle kinetic data was obtained using His-tagged human HVEM as the analyte injected at a flow rate of 50 μl/min. A eight point, two-fold dilution range from 0.78 nM to 100 nM of antigen were prepared in running buffer. For each concentration, the association phases were monitored for 180 seconds and the dissociation phase was measured for 300 seconds. Regeneration of the sensor chip surface was conducted between cycles using two injections of 10 mM Glycine-HCl, pH 1.5.

Cytotoxic assay: Nuclear RFP-stained melanoma cells were seeded for overnight incubation to allow them to attach. Then, melanoma cells were pre-incubated with 20 μg/ml of the parental or affinity matured mAbs of the invention or hIgG4 isotype control antibody (Invivogen, France) and TILs were pre-incubated with or without 20 μg/ml of anti-PD1 mAb (Nivolumab, BMS) or hIgG4 isotype control antibody (Invivogen, France). Pre-incubated TILs or medium only with anti-PD1 or hIgG4 isotype control mAb were added to the melanoma cells together with CellEvent Caspase-3/7 Green Detection Reagent (Invitrogen, USA). Plates were placed in the Incucyte ZOOM system (ESSEN Bioscience, USA) and scanned every hour. Caspase 3/7 positive events, reflecting specific killing of melanoma, were counted.

T cell activation assay by Incucyte ZOOM system: Nuclear RFP-stained melanoma cells were seeded for overnight incubation to allow them to attach. After overnight incubation, melanoma cells were incubated for one-hour at 37° C. with 20 μg/ml mAb of the invention, parental mAb or hIgG4 isotype control. Then, TILs or medium were added to the melanoma cells together with conjugated CD107a antibody. Plates were placed in the Incucyte ZOOM system and scanned every hour.

### Example 1: Affinity Maturation of Anti-HVEM Antibody

The lead antibody of WO2020222235 consisted of a heavy chain as provided in SEQ ID NO: 9 and a light chain as provided in SEQ ID NO: 10. The CDRs were in a human backbone that included an IgG4 backbone bearing the S228P mutation. This antibody bound human HVEM, as well as mouse and cynomolgus HVEM and could bind the protein in solution and on the cell surface. The antibody inhibited binding of BTLA to HVEM but did not inhibit binding of LIGHT to HVEM. Further, the antibody itself had a low-level activating effect, inducing signaling through HVEM by its binding. Most importantly the antibody augmented immune cell cytotoxicity toward cancer and was able to enhance non-autologous immune cell cytotoxicity thus enhancing standard adoptive immune cell therapy.

To improve the function of this antibody affinity maturation procedures were performed on the CDR regions of the antibody. Affinity maturation is known to improve affinity, reduce immunogenicity, overcome liabilities in structure and any combination of the above.

Affinity maturation was performed in the VH and VL CDR regions. The clones produced were sequenced and assayed for their K_{D} to HVEM extracellular domain peptide. The Fold-improvements over the K_{D} observed with the parental antibody are presented in Table 2. The affinity matured clones showed enhanced binding as compared to the parental antibody, indicated their superiority. The various mutant CDRs are combined to make antibodies with combined improved CDRs. The binding affinity of these combination antibodies are assessed.

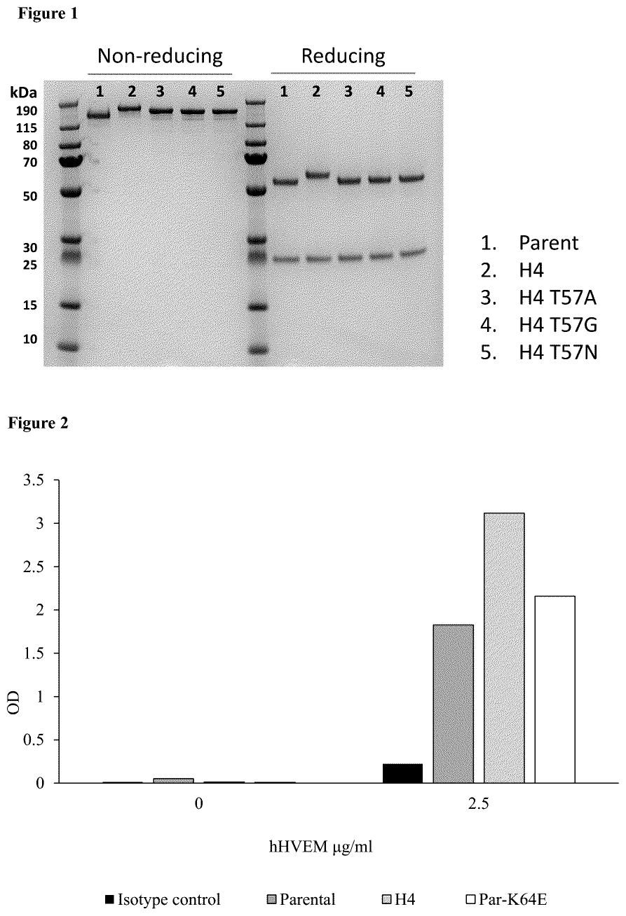

The H4 antibody was found to have the greatest improvement as compared to the parental antibody (fold increase of 9.57). However, this CDR2 contains a N-linked glycosylation site (NXS/T glycosylation site) that was found to be glycosylated in the final antibody (FIG. 1). Glycosylation creates variability in batch production of the antibody and can affect function. Therefore, several variants were generated specifically in which the asparagine at position 55 or threonine at position 57 was mutated to a different amino acid. As expected, these variants of H4 were no longer glycosylated (FIG. 1). Notably, two of these antibodies T57A and T57N had even better binding (fold increase of 20.66 and 23.51 as compared to parental) than the unmodified H4.

TABLE 2
K_{D} evaluation of antibody binding to HVEM

| | | | Fold |
|---|---|---|---|
| | | SEQ ID | improvement |
| Clone | CDR Sequence | NO: | vs parental |
| Parental-VH-CDR3 | APGDYTAYFDY | 3 | 1 |
| H1-CDR3 | ASYRNYNYGDY | 24 | 2.27 |
| H3-CDR3 | ASYDPTNYYDY | 25 | 1.54 |
| H12-CDR3 | ASYRSTNYFDY | 26 | 1.9 |
| Parental-VH-CDR2 | AISGSGGSTYYADSVKG | 2 | 1 |
| H4-CDR2 | NIYSNPNRTYYADSVEG | 22 | 9.57 |
| H4-E64K-CDR2 | NIYSNPNRTYYADSVKG | 47 | 2.15 |
| H4-N55D-CDR2 | NIYSNPDRTYYADSVEG | 57 | 3.93 |
| H4-N55E-CDR2 | NIYSNPERTYYADSVEG | 58 | 2.73 |
| H4-N55G-CDR2 | NIYSNPGRTYYADSVEG | 59 | 4.29 |
| H4-N55Q-CDR2 | NIYSNPQRTYYADSVEG | 60 | 2.59 |
| H4-N55S-CDR2 | NIYSNPSRTYYADSVEG | 61 | 2.67 |
| H4-T57A-CDR2 | NIYSNPNRAYYADSVEG | 62 | 20.66 |
| H4-T57E-CDR2 | NIYSNPNREYYADSVEG | 63 | 2.4 |
| H4-T57G-CDR2 | NIYSNPNRGYYADSVEG | 64 | 6.24 |
| H4-T57N-CDR2 | NIYSNPNRNYYADSVEG | 65 | 23.51 |
| H5-CDR2 | NINGPGNGTYYADSVEG | 23 | 5.05 |
| H6-CDR2 | GINGNGDYTYYADSVKG | 20 | 1.53 |
| H8-CDR2 | AIGGSGSGTYYADSVKG | 21 | 1.3 |
| Parental-K64E-VH-CDR2 | AISGSGGSTYYADSVEG | 48 | 3.14 |
| Parental-VL-CDR3 | QQYGSSPPYT | 6 | 1 |
| L18 (CDR3) | QQYGSYPPLT | 27 | 1 |
| L18 (CDR3)-H4 (CDR2) | | 27 + 22 | 2.57 |
| L18 (CDR3)-H5 (CDR2) | | 27 + 23 | 1.51 |

### Example 2: Further Characterization of the Affinity Matured Antibodies

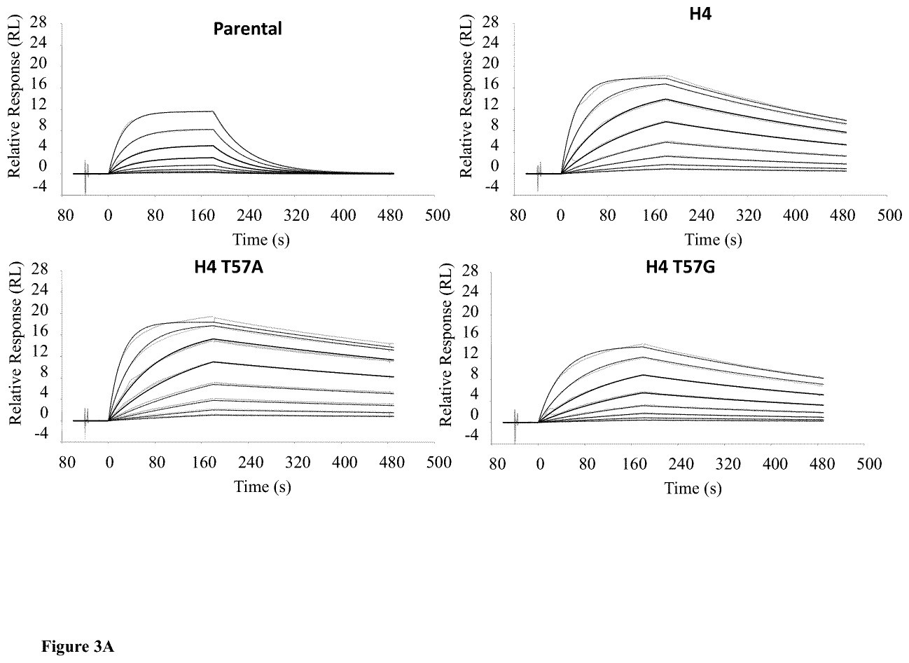

The newly generated antibodies were compared to the parental antibody to further asses their functionality. Plates were coated without protein or with 2.5 μg/ml human recombinant HVEM (hHVEM) diluted in PBS by incubation overnight at 4° C. The plates were washed, blocked with 2% BSA for an hour at room temperature and washed again. Next, four antibodies were separately tested for binding to the plates: isotype control, Parental, H4 and Par-K64E. The antibodies were diluted in blocking buffer to a concentration of 10 μg/ml and incubated for 1 hour at room temperature. The plates were then washed and incubated for 30 minutes at room temperature with peroxidase and anti-human Fc. After another wash TMB was added with stop solution. Absorbance was read at 450 nm and 570 nm. Both of the modified antibodies showed superior binding to the parental antibody (FIG. 2). Indeed, the H4 showed greatly enhanced binding and is clearly a superior option.

### Example 3: Characterization of the H4 and H4 Derivative Antibodies

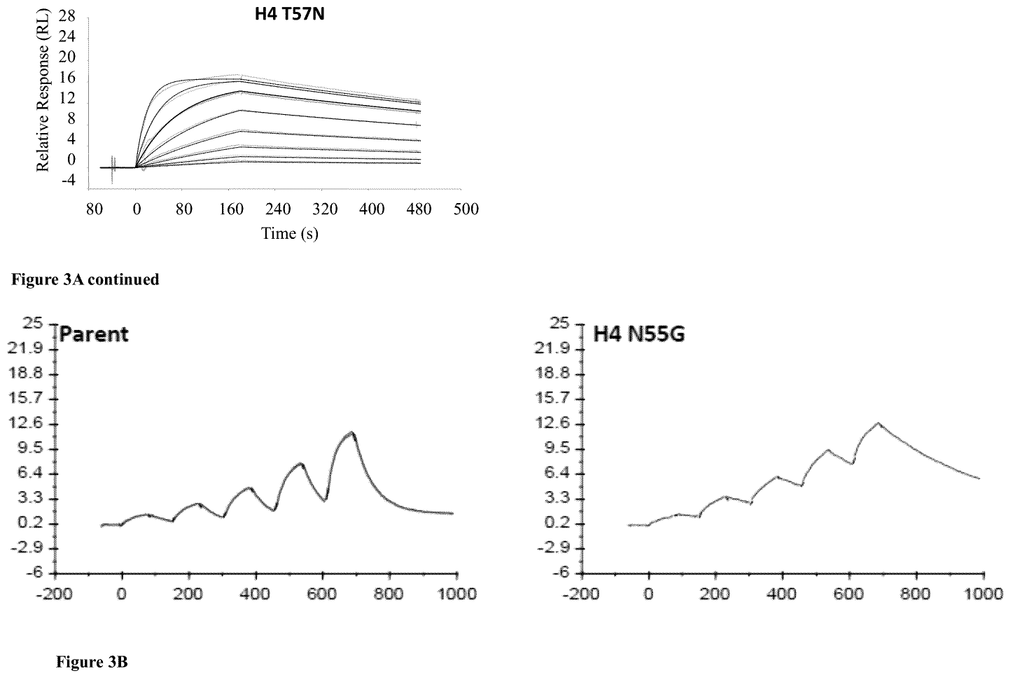

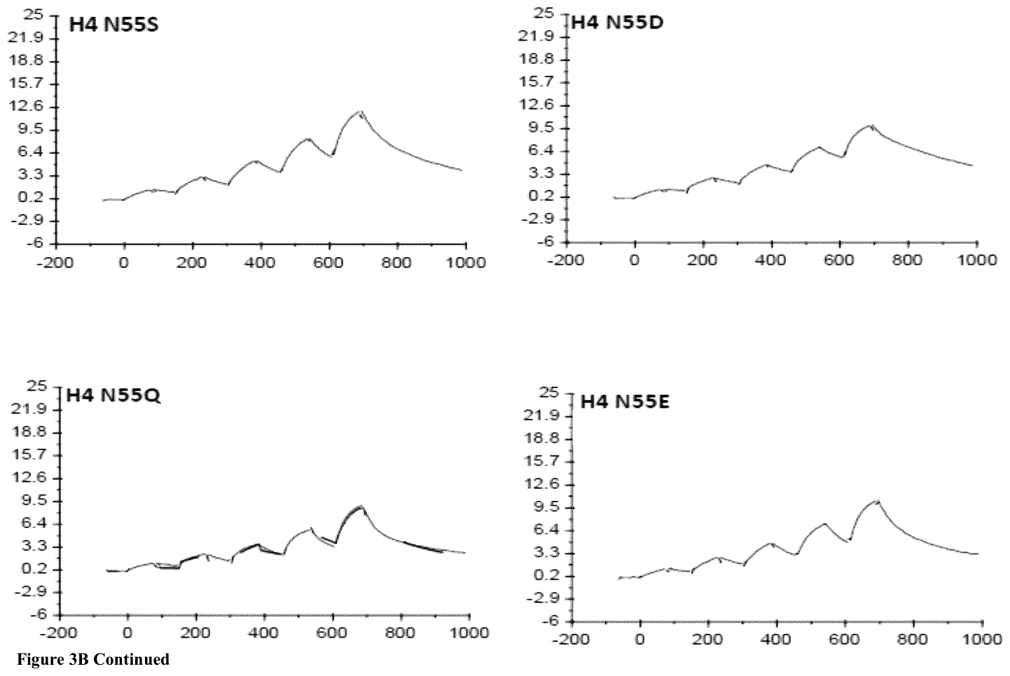

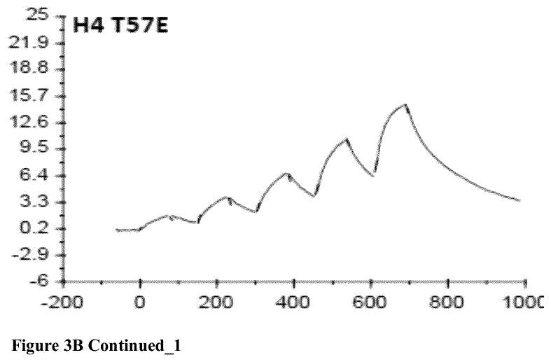

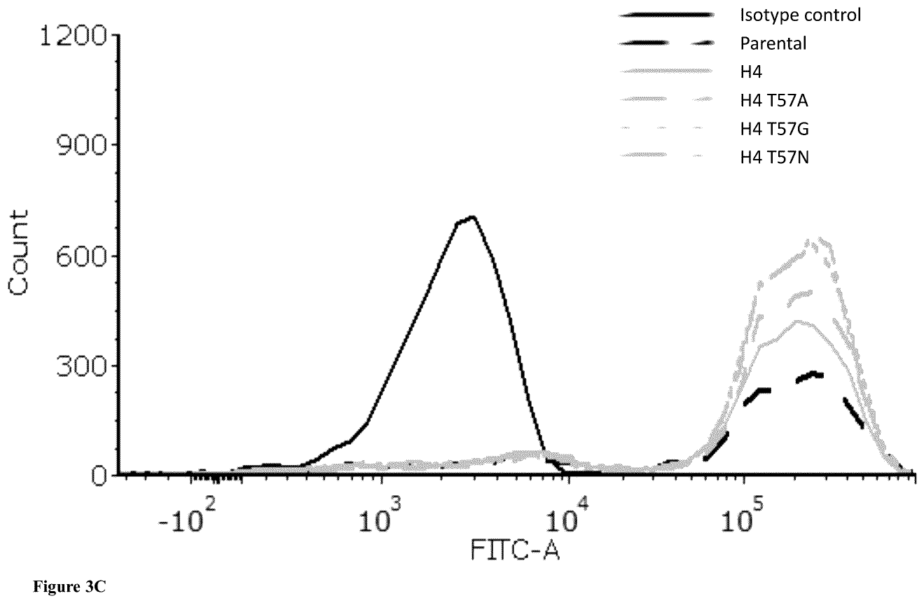

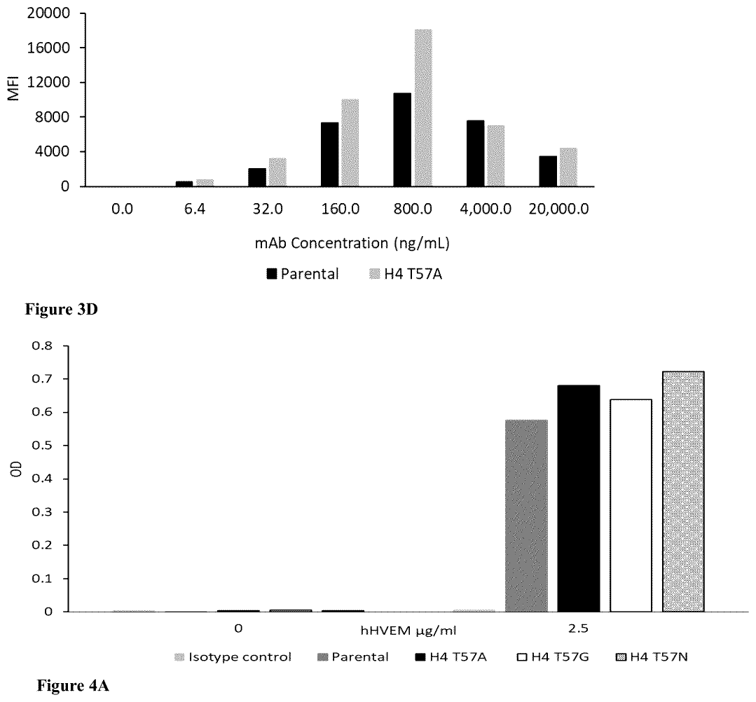

It was decided to proceed with the H4 antibody that showed the greatest HVEM binding and in particular the variants in which the a putative glycosylation site (NRT) was removed. First a multicycle kinetics Biacore affinity assay was performed to confirm the improved binding of the H4, T57A, T57G and T57N antibodies (FIG. 3A). Each purified antibody was captured to a Protein A sensor chip before increasing concentrations of antigen (hHVEM) were injected. The fold improvement in affinity was calculated by dividing the average K_{D} of the parent antibody by the average K_{D} for each variant antibody. K_{D} values and the fold improvement are summarized in Table 3. As can be seen, the H4 antibody is indeed superior to the parental antibody, and both the T57A and T57N variants are even better binders than the H4. A single-cycle kinetic Biacore assay was also performed with the H4 N55G, H4 N55S, H4 N55D, H4 N55Q, H4 N55E, and H4 T57E antibodies (FIG. 3B). K_{D} values and the fold improvement for these antibodies are summarized in Table 4. These antibodies were also found to be superior to the parental antibody.

TABLE 3
Multicycle kinetics Biacore binding assay

| Antibody | KD (M) | Fold improvement |
|---|---|---|
| Parental | 6.52E−08 | 1 |
| H4 | 4.65E−09 | 14.02 |
| H4 T57A | 2.17E−09 | 30.05 |
| H4 T57G | 7.27E−09 | 8.97 |
| H4 T57N | 1.94E−09 | 33.61 |

TABLE 4
Single-cycle kinetics Biacore binding assay

| Antibody | KD (M) | Fold improvement |
|---|---|---|
| Parental | 5.62E−08 | 1 |
| H4 N55G | 1.31E−08 | 4.29 |
| H4 N55S | 2.10E−08 | 2.67 |
| H4 N55D | 1.43E−08 | 3.93 |
| H4 N55Q | 2.17E−08 | 2.59 |
| H4 N55E | 2.06E−08 | 2.73 |
| H4 T57E | 2.34E−08 | 2.40 |

Next, the binding to HVEM on the surface of cells was examined. CHO-K1 cells were made to overexpress human HVEM (hHVEM). The cells were then incubated for an hour on ice with 20 μg/ml of the antibodies and then stained with anti-human IgG Fc biotin and anti-biotin FITC. Expression was analyzed by flow cytometry. All four antibodies were able to bind hHVEM and importantly detected surface expression (FIG. 3C). It was hypothesized that such a high concentration (20 μg/ml) was masking differences in binding between the parental antibody and the affinity matured antibodies and so binding was compared between the parental antibody and the T57A antibody at various antibody concentrations. The T57A antibody was indeed found to bind more strongly and at lower concentrations than the parental indicating that it is a superior binder of surface hHVEM (FIG. 3D).

Next, the ability of the antibodies to block interaction with BTLA was evaluated. To this end a binding ELISA system was set up. Plates were coated with recombinant hHVEM diluted in PBS at 2.5 μg/ml overnight at 4° C. Plates were washed and blocked with 2% BSA for an hour at room temperature. The various antibodies and an isotype control were diluted in blocking buffer to a concentration of 10 μg/ml. First, binding in this system was evaluated. The antibodies were incubated for 1 hour at room temperature, followed by a wash and a further incubation for 30 minutes at room temperature with peroxidase anti-human FC. Finally, following another wash, TMB was added followed by stop solution. Finally, absorbance was read at 450 nm and 570 nm.

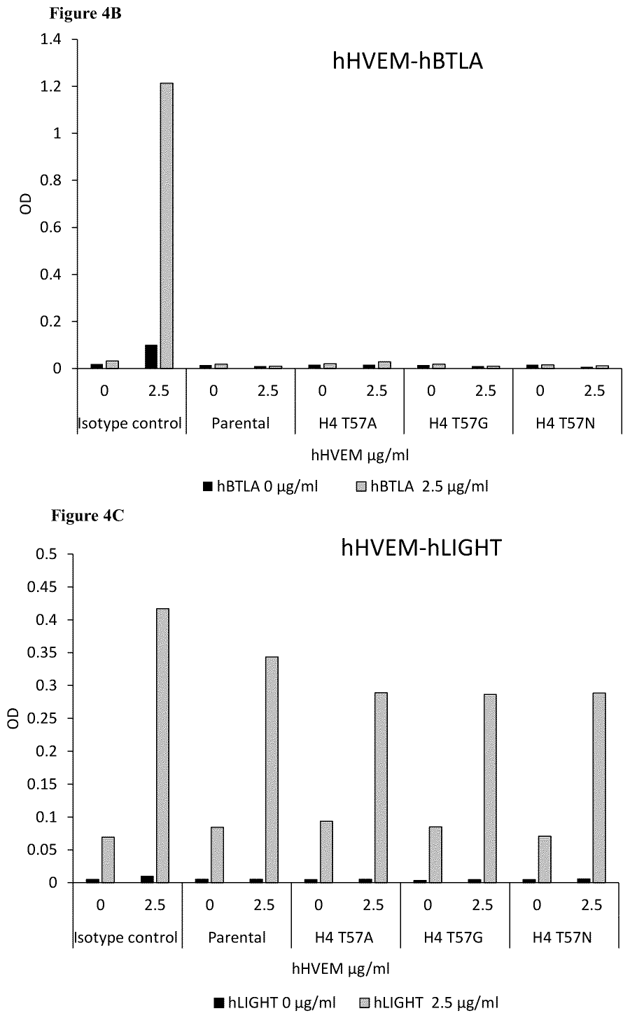

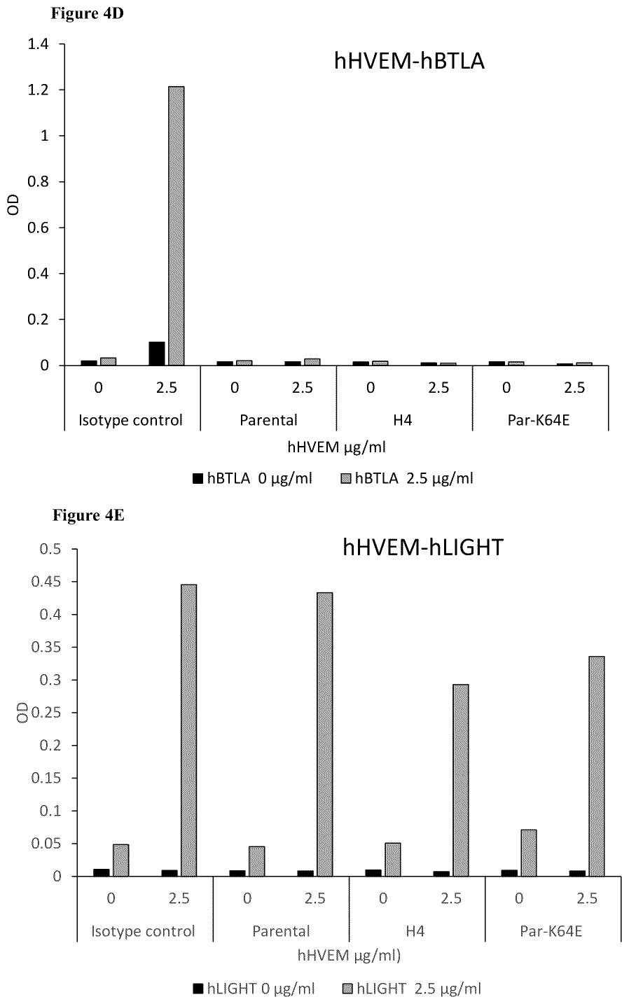

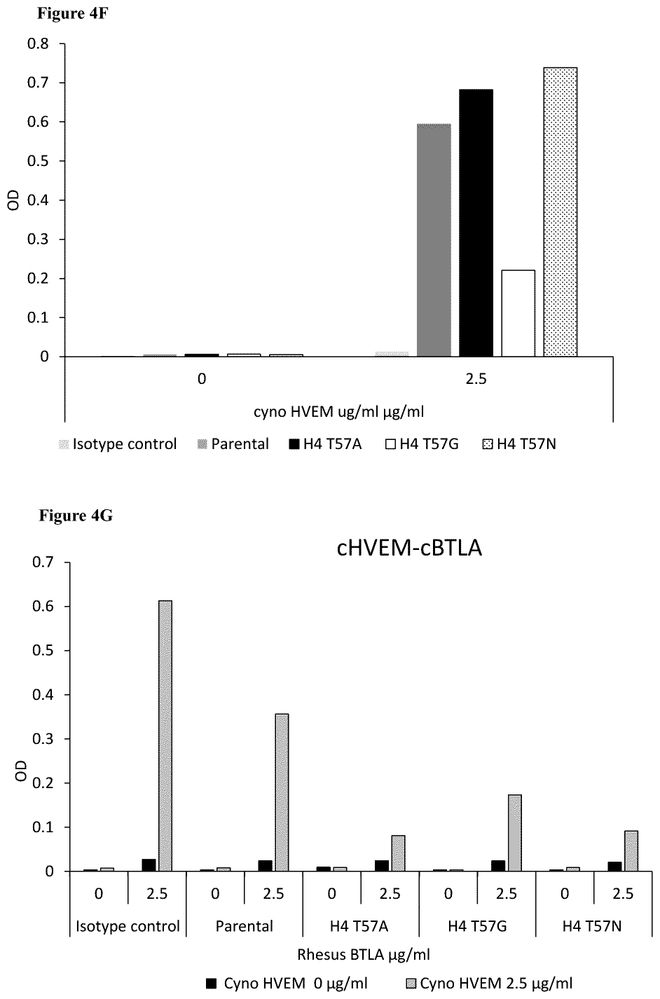

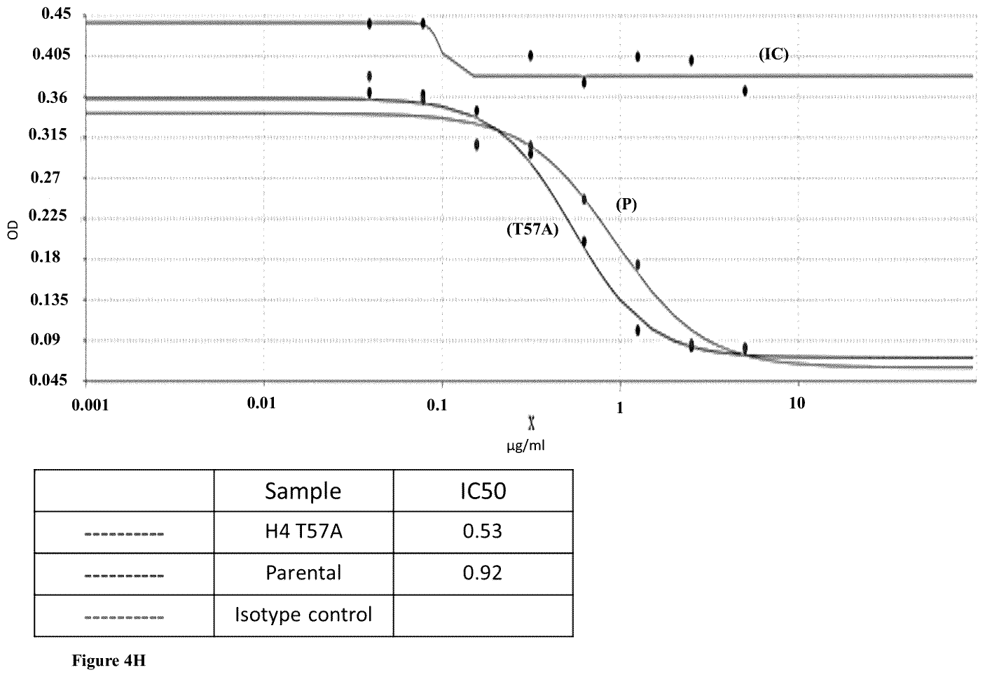

As can be seen in FIG. 4A, all 4 antibodies bind hHVEM in this setup. Additionally, all 4 antibodies were found to block hHVEM interaction with hBTLA (FIG. 4B) and did not substantially block interaction with hLIGHT (FIG. 4C) (assays performed with hBTLA-Fc and hLIGHT-His and anti-BTLA biotin or anti-LIGHT respectively). Similar results were found for the original H4 antibody and for the Par-K64E antibody (FIG. 4D-4E). Interestingly, when monkey HVEM (cHVEM) was evaluated all tested antibody showed binding to the recombinant protein (FIG. 4F), but while the parental antibody was poor at blocking cHVEM-cBTLA interaction all of the affinity matured antibodies showed better blocking (FIG. 4G).

It was hypothesized that the similarity in BTLA blocking between the parental and affinity matured antibodies was due to high antibody concentrations. As such, IC50 for blocking of hHVEM and hBTLA interaction was calculated by serial dilution of the parental antibody and the H4 T57A antibody. As can be seen in FIG. 4H, the affinity matured variant was in fact a superior blocker of hBTLA as compared to the parental. IC50 is calculated by serial dilution for the other variant antibodies and similar results are observed for the other affinity matured antibodies.

### Example 4: Functional Evaluation of the H4 and H4 Derivative Antibodies

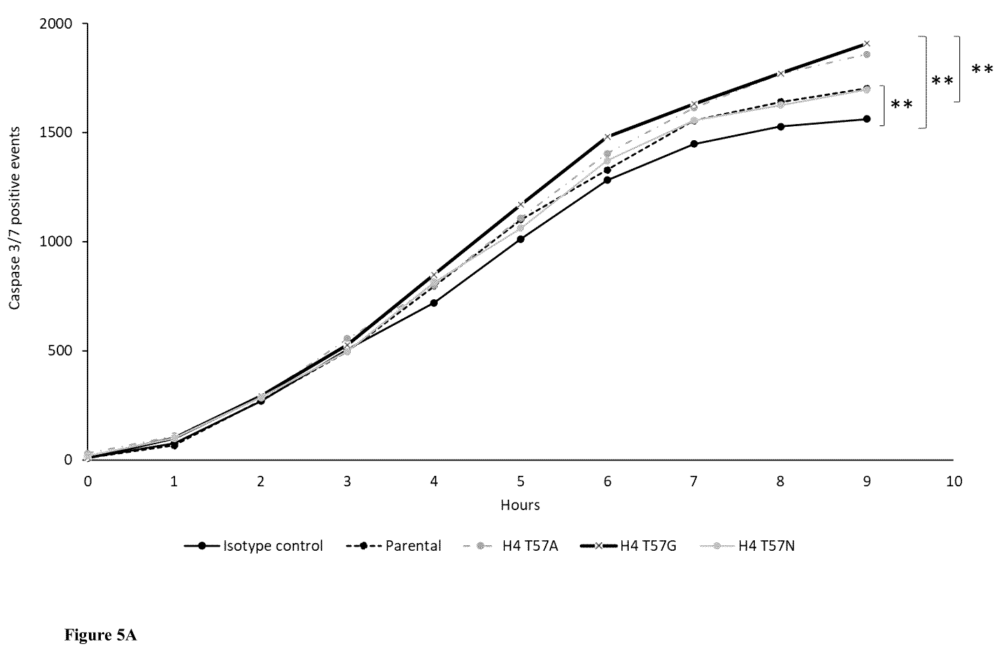

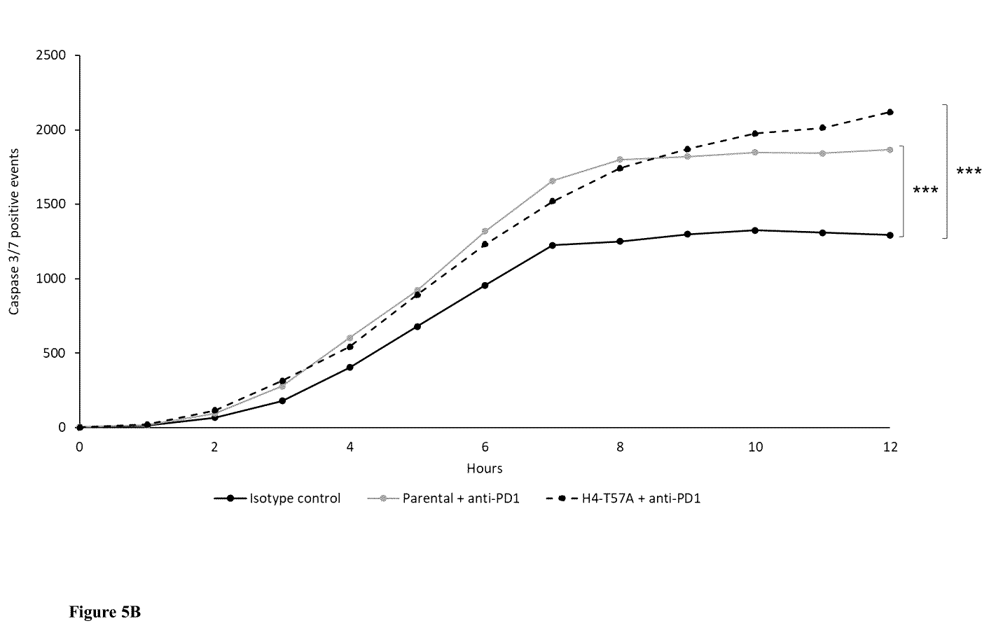

Alleviating the HVEM-BTLA T cell inhibiting axis promotes cancer cell killing by T cells. To test the cytotoxic effect of the affinity matured antibodies, RFP positive melanoma cells (HVEM positive) were cultured overnight and then incubated with the anti-HVEM antibodies (20 μg/ml) for one hour at 37° C. At the end of the hour autologous tumor-infiltrating lymphocytes (TILs) were added to the melanoma cells and cell dead events were counted using CellEvent Caspase 3/7 detection reagent and an Incucyte ZOOM system scanning every hour. After nine hours of coculture the parental antibody had produced a 9% increase in specific killing events in the cancer cells as compared to the isotype control (FIG. 5A). While the H4-T57N variant showed similar killing to the parental (9% increase), the T57A and T57G antibodies both showed a significantly greater number of killing events (19% increase, p<0.01) indicating a superior cytotoxic effect (FIG. 5A).

As the T57A variant lacks the glycosylation site, was found to bind hHVEM more than twice as strongly as H4 (also superior binding as compared to T57G) and was found to induce cell killing at an increased rate as compared to parental (also superior killing as compared to T57N) this variant antibody was selected for all further testing.

It is known that the parental antibody can enhance anti-PD-1 mediated T cell activation and thereby cancer cell killing. Therefore, the above-described killing assay was also performed in the presence of anti-PD-1 antibody. Once again, the H4 T57A antibody was found to be superior to the parental antibody, increasing specific cell killing by 64% as compared to isotype control as compared to only a 44% increase for the parental (FIG. 5B). Specific killing in the presence of anti-PD-1 antibody is also measured for the other affinity matured antibodies and similar superiority is observed.

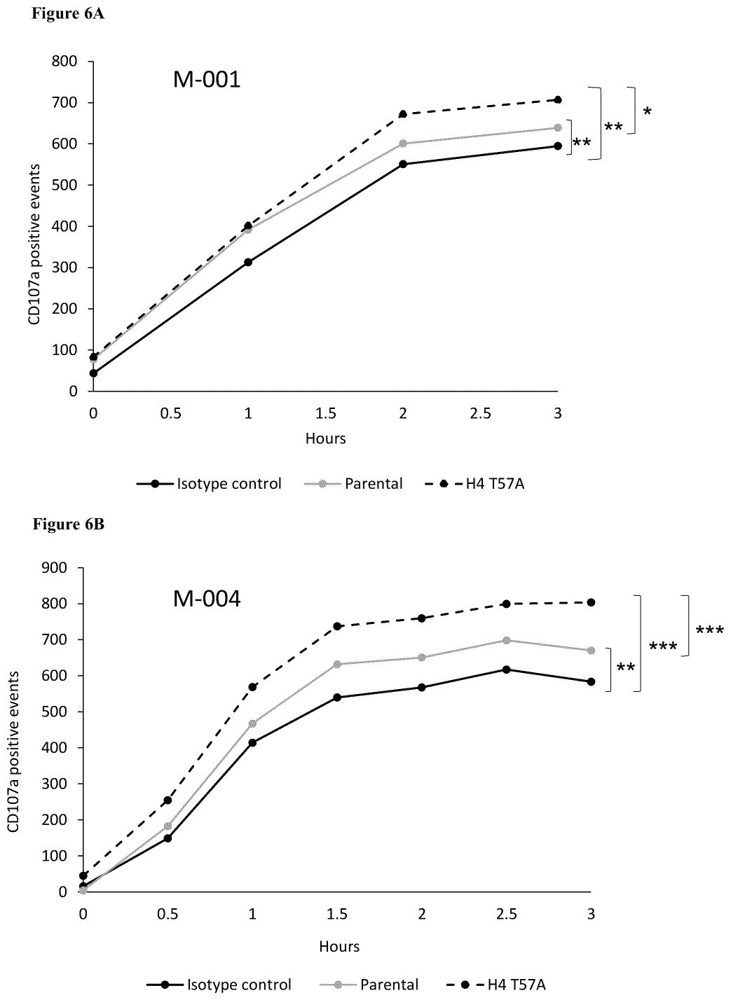

In order to better quantify the activation in the TILs caused by the anti-HVEM antibodies, the activation marker CD107a was measured in these cells when cocultured with melanoma cells in the presence of isotype control, the parental anti-HVEM antibody or the H4 T57A variant antibody. Both anti-HVEM antibodies produced a greater number of CD107a positive cells as compared to the isotype control, but the affinity matured antibody produced a significantly greater number of activated cells (FIG. 6A). When a second melanoma cell line was tested, the same trend, but with an even greater difference between the variant antibody and the parental, was observed (FIG. 6B). Activation as assessed by CD107a levels is also measured in the presence of the other affinity matured antibodies and similar superiority is observed.

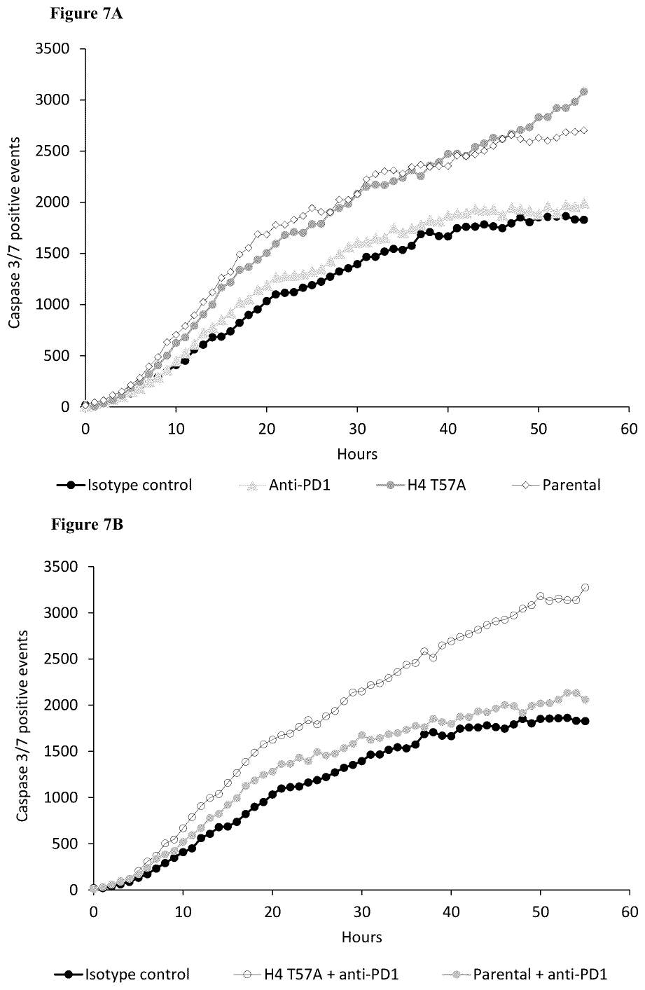

Finally, an ex vivo killing assay was also performed with primary cancer cells in a similar manner as was performed on the cell lines. Freshly isolated cells from an ovary cancer were cocultured with autologous PBMCs in the presence of isotype control, anti-PD1 antibody, the parental mAb or the H4 T57A variant antibody. Specific cancer cell killing was monitored over 55 hours. Anti-PD-1 antibody produced a very minor improvement as compared to the isotype control (9% improvement at 55 hours), whereas the parental antibody produced a 48% improvement and the H4 T57A antibody produced a 68% improvement (FIG. 7A). This again demonstrates the superiority of the affinity matured antibody. While cell killing had nearly plateaued in the coculture with the parental antibody a steady and continuous increase was observed with the variant antibody. The same assay was performed but with the anti-PD-1 combined with the parental and variant antibodies. As before, the inclusion of anti-PD-1 heightened the differences between the parental antibody and the variant. The combination of anti-PD-1 and parental antibody produced only a mild increase in specific killing as compared to anti-PD-1 and the isotype control (13% increase at 55 hours), whereas the combination of anti-PD-1 and H4 T57A antibody produced a robust 79% increase (FIG. 7B). It is thus clear that the variant antibody has a superior therapeutic effect, both alone and in combination with anti-PD-1, as compared to the parental antibody.

The other affinity matured antibodies of the invention are also tested in a similar manner to the above. Similar results, showing improved binding and efficacy as compared to the parental antibody are observed.

Additionally, murine cancer cells expressing human HVEM are implanted in a transgenic immune competent mouse expressing human BTLA and allowed to grow to form tumors. An affinity matured antibody, an anti-PD-1 antibody, combinations thereof or an IgG isotype control are administered. Tumor growth inhibition is measured. The new antibodies are found superior to the parental and at least comparable to PD-1 alone at inhibiting cancer growth. Further, the new antibodies produced an enhanced synergistic effect with anti-PD-1.

### Example 5: Diagnostic and Laboratory Use of the Variant Antibodies

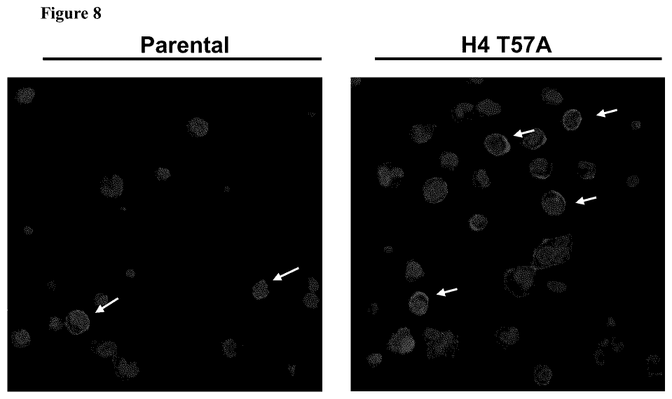

As the H4 antibody and its derivative were found to be superior binders to recombinant HVEM and bound HVEM on the cell surface it was hypothesized that the affinity matured antibodies could be used for clinical evaluation as well as laboratory staining/detection of HVEM. A formalin fixed paraffin embedded (FFPE) cell block of CHO cells overexpressing hHVEM was sliced and mounted on a slide for immunofluorescent detection. The parental antibody and the H4 T57A antibody were used along with a Cy3 (red signal) secondary antibody. Significantly more positively stained cells and with more intense staining were observed when the variant antibody was used, indicating it is a superior antibody also for diagnostic/laboratory purposes (FIG. 8).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. An antibody or antigen binding fragment thereof, comprises three heavy chain CDRs (CDR-H) and three light chain CDRs (CDR-L), wherein: CDR-H1 comprises the amino acid sequence set forth in SEQ ID NO: 1 (SYAMS), CDR-H2 comprises the amino acid sequence as set forth in SEQ ID NO: 49 (X₁IX₂X₃X₄X₅X₁₈X₇X₁₉YYADSVX₂₀G) wherein X₁ is A, G or N, X₂ is S, N, G or Y, X₃ is G or S, X₄ is S, N or P, X₅ is G or P, X₁₈ is any amino acid other than C, X₇ is S, Y, G or R, X₁₉ is any amino acid other than S or C, and X₂₀ is any amino acid, and CDR-H3 comprises the amino acid sequence as set forth in SEQ ID NO: 18 (AX₉X₁₀X₁₁X₁₂X₁₃X₁₄YX₁₅DY) wherein X₉ is P or S, X₁₀ is G or Y, X₁₁ is D or R, X₁₂ is Y, N, P or S, X₁₃ is T or Y, X₁₄ is A or N and X₁₅ is F, G or Y, CDR-L1 comprises the amino acid sequence as set forth in SEQ ID NO: 4 (RASQSVSSYLA), CDR-L2 comprises the amino acid sequence as set forth in SEQ ID NO: 5 (GASSRAT), and CDR-L3 comprises the amino acid sequence as set forth in SEQ ID NO: 19 (QQYGSX₁₆PPX₁₇T) wherein X₁₆ is S or Y and X₁₇ is Y or L; and wherein said antibody or antigen binding fragment thereof does not comprise all of: a CDR-H2 comprising the amino acid sequence as set forth in SEQ ID NO: 2 (AISGSGGSTYYADSVKG), a CDR-H3 comprising the amino acid sequence as set forth in SEQ ID NO: 3 (APGDYTAYFDY) and a CDR-L3 comprising the amino acid sequence as set forth in SEQ ID NO: 6 (QQYGSSPPYT).

2. The antibody or antigen binding fragment of claim 1, wherein: CDR-H2 comprises the amino acid sequence as set forth in SEQ ID NO: 17 (X₁IX₂X₃X₄X₅X₆X₇X₂₁YYADSVX₈G) wherein X₁ is A, G or N, X₂ is S, N, G or Y, X₃ is G or S, X₄ is S, N or P, X₅ is G or P, X₆ is G, D, S, E, Q or N, X₇ is S, Y, G or R, X₂₁ is T, A, E, G or N and X₈ is E or K; or NIYSNPNRX₂YYADSVEG (SEQ ID NO: 107) wherein X₂₃ is any amino acid other than S, T and C.

3. The antibody or antigen binding fragment thereof of claim 1, wherein X₂₀ is at least one of:
a. any amino acid other than C or S;
b. any non-positively charged amino acid; and
c. K or E.

4. (canceled)

5. (canceled)

6. (canceled)

7. The antibody or antigen binding fragment thereof of claim 1, wherein CDR-H2 comprises an amino acid sequence selected from: SEQ ID NO: 2, SEQ ID NO: 20 (GINGNGDYTYYADSVKG), SEQ ID NO: 21 (AIGGSGSGTYYADSVKG), SEQ ID NO: 22 (NIYSNPNRTYYADSVEG), SEQ ID NO: 23 (NINGPGNGTYYADSVEG), SEQ ID NO: 47 (NIYSNPNRTYYADSVKG), SEQ ID NO: 48: (AISGSGGSTYYADSVEG), SEQ ID NO: 57 (NIYSNPDRTYYADSVEG), SEQ ID NO: 58 (NIYSNPERTYYADSVEG), SEQ ID NO: 59 (NIYSNPGRTYYADSVEG), SEQ ID NO: 60 (NIYSNPQRTYYADSVEG), SEQ ID NO: 61 (NIYSNPSRTYYADSVEG), SEQ ID NO: 62 (NIYSNPNRAYYADSVEG), SEQ ID NO: 63 (NIYSNPNREYYADSVEG), SEQ ID NO: 64 (NIYSNPNRGYYADSVEG) and SEQ ID NO: 65 (NIYSNPNRNYYADSVEG).

8. The antibody or antigen binding fragment thereof of claim 1, wherein CDR-H3 comprises an amino acid sequence selected from: SEQ ID NO: 3, SEQ ID NO: 24 (ASYRNYNYGDY), SEQ ID NO: 25 (ASYDPTNYYDY) and SEQ ID NO: 26 (ASYRSTNYFDY).

9. The antibody or antigen binding fragment thereof of claim 1, wherein CDR-L3 comprises an amino acid sequence selected from: SEQ ID NO: 6 and SEQ ID NO: 27 (QQYGSYPPLT).

10. The antibody or antigen binding fragment thereof of claim 1, wherein: CDR-H1 comprises the amino acid sequence set forth in SEQ ID NO: 1, CDR-H2 comprises an amino acid sequence selected from SEQ ID NO: 20, SEQ ID NO: 21, SEQ ID NO: 22, SEQ ID NO: 23, SEQ ID NO: 47, SEQ ID NO: 48, SEQ ID NO: 57, SEQ ID NO: 58, SEQ ID NO: 59, SEQ ID NO: 60, SEQ ID NO: 61, SEQ ID NO: 62, SEQ ID NO: 63, SEQ ID NO: 64 and SEQ ID NO: 65, CDR-H3 comprises the amino acid sequence as set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6.

11. The antibody or antigen binding fragment thereof of claim 1, wherein: CDR-H1 comprises the amino acid sequence set forth in SEQ ID NO: 1, CDR-H2 comprises the amino acid sequence set forth in SEQ ID NO: 2, CDR-H3 comprises an amino acid sequence selected from SEQ ID NO: 24, SEQ ID NO: 25 and SEQ ID NO: 26, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 6.

12. The antibody or antigen binding fragment thereof of claim 1, wherein: CDR-H1 comprises the amino acid sequence set forth in SEQ ID NO: 1, CDR-H2 comprises the amino acid sequence set forth in SEQ ID NO: 2, CDR-H3 comprises the amino acid sequence set forth in SEQ ID NO: 3, CDR-L1 comprises the amino acid sequence as set forth in SEQ ID NO: 4, CDR-L2 comprises the amino acid sequence as set forth in SEQ ID NO: 5, and CDR-L3 comprises the amino acid sequence as set forth in SEQ ID NO: 27.

13. The antibody or antigen binding fragment thereof of claim 1, comprising a heavy chain comprising a sequence selected from
(SEQ ID NO: 7)
QVQLVQSGGGLVQPGGSLRLSCAASGFTFSSYAMSWVRQAPGKGLEWV
SAISGSGGSTYYADSVKGRFTISRDNSKNTLYLQMNSLRAEDTAVYYCAK
APGDYTAYFDYWGQGTLVTVSS,
(SEQ ID NO: 28)
QVQLVQSGGGLVQPGGSLRLSCAASGFTFSSYAMSWVRQAPGKGLEWV
SGINGNGDYTYYADSVKGRFTISRDNSKNTLYLQMNSLRAEDTAVYYCA
KAPGDYTAYFDYWGQGTLVTVSS,
(SEQ ID NO: 29)
QVQLVQSGGGLVQPGGSLRLSCAASGFTFSSYAMSWVRQAPGKGLEWV
SAIGGSGSGTYYADSVKGRFTISRDNSKNTLYLQMNSLRAEDTAVYYCA
KAPGDYTAYFDYWGQGTLVTVSS,
(SEQ ID NO: 30)
QVQLVQSGGGLVQPGGSLRLSCAASGFTFSSYAMSWVRQAPGKGLEWV
SNIYSNPNRTYYADSVEGRFTISRDNSKNTLYLQMNSLRAEDTAVYYCAK
APGDYTAYFDYWGQGTLVTVSS,
(SEQ ID NO: 31)
QVQLVQSGGGLVQPGGSLRLSCAASGFTFSSYAMSWVRQAPGKGLEWV
SNINGPGNGTYYADSVEGRFTISRDNSKNTLYLQMNSLRAEDTAVYYCA
KAPGDYTAYFDYWGQGTLVTVSS,
(SEQ ID NO: 50)
QVQLVQSGGGLVQPGGSLRLSCAASGFTFSSYAMSWVRQAPGKGLEWV
SNIYSNPNRTYYADSVKGRFTISRDNSKNTLYLQMNSLRAEDTAVYYCA
KAPGDYTAYFDYWGQGTLVTVSS,
(SEQ ID NO: 51)
QVQLVQSGGGLVQPGGSLRLSCAASGFTFSSYAMSWVRQAPGKGLEWV
SAISGSGGSTYYADSVEGRFTISRDNSKNTLYLQMNSLRAEDTAVYYCAK
APGDYTAYFDYWGQGTLVTVSS,
(SEQ ID NO: 66)
QVQLVQSGGGLVQPGGSLRLSCAASGFTFSSYAMSWVRQAPGKGLEWV
SNIYSNPDRTYYADSVEGRFTISRDNSKNTLYLQMNSLRAEDTAVYYCAK
APGDYTAYFDYWGQGTLVTVSS,
(SEQ ID NO: 67)
QVQLVQSGGGLVQPGGSLRLSCAASGFTFSSYAMSWVRQAPGKGLEWV
SNIYSNPERTYYADSVEGRFTISRDNSKNTLYLQMNSLRAEDTAVYYCAK
APGDYTAYFDYWGQGTLVTVSS,
(SEQ ID NO: 68)
QVQLVQSGGGLVQPGGSLRLSCAASGFTFSSYAMSWVRQAPGKGLEWV
SNIYSNPGRTYYADSVEGRFTISRDNSKNTLYLQMNSLRAEDTAVYYCAK
APGDYTAYFDYWGQGTLVTVSS,
(SEQ ID NO: 69)
QVQLVQSGGGLVQPGGSLRLSCAASGFTFSSYAMSWVRQAPGKGLEWV
SNIYSNPQRTYYADSVEGRFTISRDNSKNTLYLQMNSLRAEDTAVYYCAK
APGDYTAYFDYWGQGTLVTVSS,
(SEQ ID NO: 70)
QVQLVQSGGGLVQPGGSLRLSCAASGFTFSSYAMSWVRQAPGKGLEWV
SNIYSNPSRTYYADSVEGRFTISRDNSKNTLYLQMNSLRAEDTAVYYCAK
APGDYTAYFDYWGQGTLVTVSS,
(SEQ ID NO: 71)
QVQLVQSGGGLVQPGGSLRLSCAASGFTFSSYAMSWVRQAPGKGLEWV
SNIYSNPNRAYYADSVEGRFTISRDNSKNTLYLQMNSLRAEDTAVYYCA
KAPGDYTAYFDYWGQGTLVTVSS,
(SEQ ID NO: 72)
QVQLVQSGGGLVQPGGSLRLSCAASGFTFSSYAMSWVRQAPGKGLEWV
SNIYSNPNREYYADSVEGRFTISRDNSKNTLYLQMNSLRAEDTAVYYCAK
APGDYTAYFDYWGQGTLVTVSS,
(SEQ ID NO: 73)
QVQLVQSGGGLVQPGGSLRLSCAASGFTFSSYAMSWVRQAPGKGLEWV
SNIYSNPNRGYYADSVEGRFTISRDNSKNTLYLQMNSLRAEDTAVYYCA
KAPGDYTAYFDYWGQGTLVTVSS,
(SEQ ID NO: 74)
QVQLVQSGGGLVQPGGSLRLSCAASGFTFSSYAMSWVRQAPGKGLEWV
SNIYSNPNRNYYADSVEGRFTISRDNSKNTLYLQMNSLRAEDTAVYYCA
KAPGDYTAYFDYWGQGTLVTVSS,
(SEQ ID NO: 32)
QVQLVQSGGGLVQPGGSLRLSCAASGFTFSSYAMSWVRQAPGKGLEWV
SAISGSGGSTYYADSVKGRFTISRDNSKNTLYLQMNSLRAEDTAVYYCAK
ASYRNYNYGDYWGQGTLVTVSS,
(SEQ ID NO: 33)
QVQLVQSGGGLVQPGGSLRLSCAASGFTFSSYAMSWVRQAPGKGLEWV
SAISGSGGSTYYADSVKGRFTISRDNSKNTLYLQMNSLRAEDTAVYYCAK
ASYDPTNYYDYWGQGTLVTVSS,
and
(SEQ ID NO: 34)
QVQLVQSGGGLVQPGGSLRLSCAASGFTFSSYAMSWVRQAPGKGLEWV
SAISGSGGSTYYADSVKGRFTISRDNSKNTLYLQMNSLRAEDTAVYYCAK
ASYRSTNYFDYWGQGTLVTVSS.

14. The antibody or antigen binding fragment thereof of claim 1, comprising a light chain comprising a sequence selected from:
(SEQ ID NO: 8)
ELVLTQSPATLSLSPGERATLSCRASQSVSSYLAWYQQKPGQAPRLLIYG
ASSRATGIPDRFSGSGSGTDFTLTISRLEPEDFAVYYCQQYGSSPPYTFG
QGTKVEIK
and
(SEQ ID NO: 35)
ELVLTQSPATLSLSPGERATLSCRASQSVSSYLAWYQQKPGQAPRLLIYG
ASSRATGIPDRFSGSGSGTDFTLTISRLEPEDFAVYYCQQYGSYPPLTFG
QGTKVEIK.

15. The antibody or antigen binding fragment thereof of any-one claim 1, comprising a heavy chain comprising an amino acid sequence selected from SEQ ID NO: 85-103, a light chain comprising an amino acid sequence selected from SEQ ID NO: 104 and SEQ ID NO: 105 or both.

16. (canceled)

17. The antibody or antigen binding fragment thereof of claim 1, comprising a heavy chain comprising SEQ ID NO: 97 and a light chain comprising SEQ ID NO: 104.

18. A pharmaceutical composition comprising an antibody or antigen binding fragment thereof of claim 1 and a pharmaceutically acceptable carrier, excipient or adjuvant.

19. A method of treating a disease or condition characterized by HVEM positive cells, wherein said disease or condition is an HVEM positive cancer or precancerous lesion or an infectious disease and wherein said infected cells comprise HVEM expression, in a subject in need thereof, the method comprising administering to the subject the pharmaceutical composition of claim 18, thereby treating said disease or condition.

20. The method of claim 19, further comprising at least one of:
a. inhibition or blockade of a non-HVEM immune checkpoint protein;
b. administering an anti-PD-1/PD-L1 based immunotherapy;
c. administering adoptive cell therapy;
d. administering adoptive tumor infiltrating lymphocyte (TIL) therapy; and
e. administering an adoptive cell therapy comprising a chimeric antigen receptor (CAR) expressing immune cell, wherein said CAR targets a non-HVEM protein on a surface of said HVEM expressing cells.

21. (canceled)

22. (canceled)

23. (canceled)

24. (canceled)

25. (canceled)

26. (canceled)

27. A method of determining suitability of a subject to be treated by a method of claim 19, comprising obtaining a disease sample from said subject and determining HVEM levels in said sample, wherein positive expression of HVEM indicates the subject is suitable for a method of treatment of claim 19, and wherein positive expression comprises an elevated HVEM level as compared to a healthy sample or predetermined threshold, thereby determining suitability of a subject to be treated by a method of claim 19.

28. (canceled)

29. A method of detecting HVEM in a sample, the method comprising contacting said sample with an antibody or antigen binding fragment thereof of claim 1, thereby detecting HVEM.

30. A nucleic acid molecule encoding an antibody or antigen binding fragment thereof of claim 1.

31. A kit comprising, the pharmaceutical composition of claim 18 and at least one of:
a. an anti-PD-1/PD-L1 based immunotherapy;
b. a label stating the pharmaceutical composition of the invention is for use with an anti-PD-1/PD-L1 based immunotherapy; and
c. a secondary detection molecule for detecting said at least one antibody or antigen binding fragment thereof.


SEQUENCE LISTING (120 sequences)

SEQ ID NO: 1 - protein, 5 aa, synthetic construct
  REGION 1..5: note = Synthetic
SYAMS

SEQ ID NO: 2 - protein, 17 aa, synthetic construct
  REGION 1..17: note = Synthetic
AISGSGGSTYYADSVKG

SEQ ID NO: 3 - protein, 11 aa, synthetic construct
  REGION 1..11: note = Synthetic
APGDYTAYFDY

SEQ ID NO: 4 - protein, 11 aa, synthetic construct
  REGION 1..11: note = Synthetic
RASQSVSSYLA

SEQ ID NO: 5 - protein, 7 aa, synthetic construct
  REGION 1..7: note = Synthetic
GASSRAT

SEQ ID NO: 6 - protein, 10 aa, synthetic construct
  REGION 1..10: note = Synthetic
QQYGSSPPYT

SEQ ID NO: 7 - protein, 120 aa, synthetic construct
  REGION 1..120: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAPGDYTAYFDYWGQGTLVTVSS

SEQ ID NO: 8 - protein, 108 aa, synthetic construct
  REGION 1..108: note = Synthetic
ELVLTQSPATLSLSPGERATLSCRASQSVSSYLAWYQQKPGQAPRLLIYGASSRATGIPDRFSGSGSGTDFTLTISRLEPEDFAVYYCQQYGSSPPYTFGQGTKVEIK

SEQ ID NO: 9 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 234 aa, synthetic construct
  REGION 1..234: note = Synthetic
MGWSCIILFLVATATGVHSELVLTQSPATLSLSPGERATLSCRASQSVSSYLAWYQQKPGQAPRLLIYGASSRATGIPDRFSGSGSGTDFTLTISRLEPEDFAVYYCQQYGSSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, synthetic construct
  REGION 1..8: note = Synthetic
GFTFSSYA

SEQ ID NO: 12 - protein, 8 aa, synthetic construct
  REGION 1..8: note = Synthetic
ISGSGGST

SEQ ID NO: 13 - protein, 13 aa, synthetic construct
  REGION 1..13: note = Synthetic
AKAPGDYTAYFDY

SEQ ID NO: 14 - protein, 6 aa, synthetic construct
  REGION 1..6: note = Synthetic
QSVSSY

SEQ ID NO: 15 - skipped

SEQ ID NO: 16 - protein, 10 aa, synthetic construct
  REGION 1..10: note = Synthetic
QQYGSSPPYT

SEQ ID NO: 17 - protein, 17 aa, synthetic construct
  REGION 1..17: note = Synthetic
  VAR_SEQ 1: note = X1 - X1 is A, G or N
  VAR_SEQ 3: note = X2 - X2 is S, N, G or Y
  VAR_SEQ 4: note = X3 - X3 is G or S
  VAR_SEQ 5: note = X4 - X4 is S, N or P
  VAR_SEQ 6: note = X5 - X5 is G or P
  VAR_SEQ 7: note = X6 - X6 is G, D, S, E, Q or N
  VAR_SEQ 8: note = X7 - X7 is S, Y, G or R
  VAR_SEQ 9: note = X21 - X21 is T, A, E, G or N
  VAR_SEQ 16: note = X8 - X8 is E or K
XIXXXXXXXYYADSVXG

SEQ ID NO: 18 - protein, 11 aa, synthetic construct
  REGION 1..11: note = Synthetic
  VAR_SEQ 2: note = X9 - X9 is P or S
  VAR_SEQ 3: note = X10 - X10 is G or Y
  VAR_SEQ 4: note = X11 - X11 is D or R
  VAR_SEQ 5: note = X12 - X12 is Y, N, P or S
  VAR_SEQ 6: note = X13 - X13 is T or Y
  VAR_SEQ 7: note = X14 - X14 is A or N
  VAR_SEQ 9: note = X15 - X15 is F, G or Y
AXXXXXXYXDY

SEQ ID NO: 19 - protein, 10 aa, synthetic construct
  REGION 1..10: note = Synthetic
  SITE 6: note = X16 - X16 is S or Y
  SITE 9: note = X17 - X17 is Y or L
QQYGSXPPXT

SEQ ID NO: 20 - protein, 17 aa, synthetic construct
  REGION 1..17: note = Synthetic
GINGNGDYTYYADSVKG

SEQ ID NO: 21 - protein, 17 aa, synthetic construct
  REGION 1..17: note = Synthetic
AIGGSGSGTYYADSVKG

SEQ ID NO: 22 - protein, 17 aa, synthetic construct
  REGION 1..17: note = Synthetic
NIYSNPNRTYYADSVEG

SEQ ID NO: 23 - protein, 17 aa, synthetic construct
  REGION 1..17: note = Synthetic
NINGPGNGTYYADSVEG

SEQ ID NO: 24 - protein, 11 aa, synthetic construct
  REGION 1..11: note = Synthetic
ASYRNYNYGDY

SEQ ID NO: 25 - protein, 11 aa, synthetic construct
  REGION 1..11: note = Synthetic
ASYDPTNYYDY

SEQ ID NO: 26 - protein, 11 aa, synthetic construct
  REGION 1..11: note = Synthetic
ASYRSTNYFDY

SEQ ID NO: 27 - protein, 10 aa, synthetic construct
  REGION 1..10: note = Synthetic
QQYGSYPPLT

SEQ ID NO: 28 - protein, 120 aa, synthetic construct
  REGION 1..120: note = Synthetic
QVQLVQSGGGLVQPGGSLRLSCAASGFTFSSYAMSWVRQAPGKGLEWVSGINGNGDYTYYADSVKGRFTISRDNSKNTLYLQMNSLRAEDTAVYYCAKAPGDYTAYFDYWGQGTLVTVSS

SEQ ID NO: 29 - protein, 120 aa, synthetic construct
  REGION 1..120: note = Synthetic
QVQLVQSGGGLVQPGGSLRLSCAASGFTFSSYAMSWVRQAPGKGLEWVSAIGGSGSGTYYADSVKGRFTISRDNSKNTLYLQMNSLRAEDTAVYYCAKAPGDYTAYFDYWGQGTLVTVSS

SEQ ID NO: 30 - protein, 120 aa, synthetic construct
  REGION 1..120: note = Synthetic
QVQLVQSGGGLVQPGGSLRLSCAASGFTFSSYAMSWVRQAPGKGLEWVSNIYSNPNRTYYADSVEGRFTISRDNSKNTLYLQMNSLRAEDTAVYYCAKAPGDYTAYFDYWGQGTLVTVSS

SEQ ID NO: 31 - protein, 120 aa, synthetic construct
  REGION 1..120: note = Synthetic
QVQLVQSGGGLVQPGGSLRLSCAASGFTFSSYAMSWVRQAPGKGLEWVSNINGPGNGTYYADSVEGRFTISRDNSKNTLYLQMNSLRAEDTAVYYCAKAPGDYTAYFDYWGQGTLVTVSS

SEQ ID NO: 32 - protein, 120 aa, synthetic construct
  REGION 1..120: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYRNYNYGDYWGQGTLVTVSS

SEQ ID NO: 33 - protein, 120 aa, synthetic construct
  REGION 1..120: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYDPTNYYDYWGQGTLVTVSS

SEQ ID NO: 34 - protein, 120 aa, synthetic construct
  REGION 1..120: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYRSTNYFDYWGQGTLVTVSS

SEQ ID NO: 35 - protein, 108 aa, synthetic construct
  REGION 1..108: note = Synthetic
ELVLTQSPATLSLSPGERATLSCRASQSVSSYLAWYQQKPGQAPRLLIYGASSRATGIPDRFSGSGSGTDFTLTISRLEPEDFAVYYCQQYGSYPPLTFGQGTKVEIK

SEQ ID NO: 36 - protein, 19 aa, synthetic construct
  REGION 1..19: note = Synthetic
MGWSCIILFLVATATGVHS

SEQ ID NO: 37 - protein, 327 aa, synthetic construct
  REGION 1..327: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 38 - protein, 107 aa, synthetic construct [sequence omitted]

SEQ ID NO: 39 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSGINGNGDYTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 40 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAIGGSGSGTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPNRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNINGPGNGTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYRNYNYGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYDPTNYYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYRSTNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 234 aa, synthetic construct
  REGION 1..234: note = Synthetic
MGWSCIILFLVATATGVHSELVLTQSPATLSLSPGERATLSCRASQSVSSYLAWYQQKPGQAPRLLIYGASSRATGIPDRFSGSGSGTDFTLTISRLEPEDFAVYYCQQYGSYPPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 17 aa, synthetic construct
  REGION 1..17: note = Synthetic
NIYSNPNRTYYADSVKG

SEQ ID NO: 48 - protein, 17 aa, synthetic construct
  REGION 1..17: note = Synthetic
AISGSGGSTYYADSVEG

SEQ ID NO: 49 - protein, 17 aa, synthetic construct
  REGION 1..17: note = Synthetic
  VAR_SEQ 1: note = X1 - X1 is A, G or N
  VAR_SEQ 3: note = X2 - X2 is S, N, G or Y
  VAR_SEQ 4: note = X3 - X3 is G or S
  VAR_SEQ 5: note = X4 - X4 is S, N or P
  VAR_SEQ 6: note = X5 - X5 is G or P
  VAR_SEQ 7: note = X18 - X18 is any amino acid other than C
  VAR_SEQ 8: note = X7 - X7 is S, Y, G or R
  VAR_SEQ 9: note = X19 - X19 is any amino acid other than S and C
  VAR_SEQ 16: note = X20 - X20 is any amino acid
XIXXXXXXXYYADSVXG

SEQ ID NO: 50 - protein, 120 aa, synthetic construct
  REGION 1..120: note = Synthetic
QVQLVQSGGGLVQPGGSLRLSCAASGFTFSSYAMSWVRQAPGKGLEWVSNIYSNPNRTYYADSVKGRFTISRDNSKNTLYLQMNSLRAEDTAVYYCAKAPGDYTAYFDYWGQGTLVTVSS

SEQ ID NO: 51 - protein, 120 aa, synthetic construct
  REGION 1..120: note = Synthetic
QVQLVQSGGGLVQPGGSLRLSCAASGFTFSSYAMSWVRQAPGKGLEWVSAISGSGGSTYYADSVEGRFTISRDNSKNTLYLQMNSLRAEDTAVYYCAKAPGDYTAYFDYWGQGTLVTVSS

SEQ ID NO: 52 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPNRTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSAISGSGGSTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 447 aa, synthetic construct
  REGION 1..447: note = Synthetic
  VAR_SEQ 50: note = X1 - X1 is A, G or N
  VAR_SEQ 52: note = X2 - X2 is S, N, G or Y
  VAR_SEQ 53: note = X3 - X3 is G or S
  VAR_SEQ 54: note = X4 - X4 is S, N or P
  VAR_SEQ 55: note = X5 - X5 is G or P
  VAR_SEQ 56: note = X6 - X6 is G, D, S, or N
  VAR_SEQ 57: note = X7 - X7 is S, Y, G or R
  VAR_SEQ 65: note = X8 - X8 is E or K
QVQLVQSGGGLVQPGGSLRLSCAASGFTFSSYAMSWVRQAPGKGLEWVSXIXXXXXXTYYADSVXGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 55 - protein, 447 aa, synthetic construct
  REGION 1..447: note = Synthetic
  VAR_SEQ 50: note = X1 - X1 is A, G or N
  VAR_SEQ 52: note = X2-X2 is S, N, G or Y
  VAR_SEQ 53: note = X3 - X3 is G or S
  VAR_SEQ 54: note = X4 - X4 is S, N or P
  VAR_SEQ 55: note = X5-X5 is G or P
  VAR_SEQ 56: note = X18-X18 is any amino acid other than C
  VAR_SEQ 57: note = X7 - X7 is S, Y, G or R
  VAR_SEQ 58: note = X19 - X19 is any amino acid other than S and C
  VAR_SEQ 65: note = X20 - X20 is any amino acid
  VAR_SEQ 74: note = X5 - X5 is G or P
QVQLVQSGGGLVQPGGSLRLSCAASGFTFSSYAMSWVRQAPGKGLEWVSXIXXXXXXXYYADSVXGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 56 - protein, 447 aa, synthetic construct
  REGION 1..447: note = Synthetic
  VAR_SEQ 100: note = X9 - X9 is P or S
  VAR_SEQ 101: note = X10 - X10 is G or Y
  VAR_SEQ 102: note = X11 - X11 is D or R
  VAR_SEQ 103: note = X12 - X12 is Y, N, P or S
  VAR_SEQ 104: note = X13 - X13 is T or Y
  VAR_SEQ 105: note = X14 - X14 is A or N
  VAR_SEQ 107: note = X15 - X15 is F, G or Y
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAXXXXXXYXDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 17 aa, synthetic construct
  REGION 1..17: note = Synthetic
NIYSNPDRTYYADSVEG

SEQ ID NO: 58 - protein, 17 aa, synthetic construct
  REGION 1..17: note = Synthetic
NIYSNPERTYYADSVEG

SEQ ID NO: 59 - protein, 17 aa, synthetic construct
  REGION 1..17: note = Synthetic
NIYSNPGRTYYADSVEG

SEQ ID NO: 60 - protein, 17 aa, synthetic construct
  REGION 1..17: note = Synthetic
NIYSNPQRTYYADSVEG

SEQ ID NO: 61 - protein, 17 aa, synthetic construct
  REGION 1..17: note = Synthetic
NIYSNPSRTYYADSVEG

SEQ ID NO: 62 - protein, 17 aa, synthetic construct
  REGION 1..17: note = Synthetic
NIYSNPNRAYYADSVEG

SEQ ID NO: 63 - protein, 17 aa, synthetic construct
  REGION 1..17: note = Synthetic
NIYSNPNREYYADSVEG

SEQ ID NO: 64 - protein, 17 aa, synthetic construct
  REGION 1..17: note = Synthetic
NIYSNPNRGYYADSVEG

SEQ ID NO: 65 - protein, 17 aa, synthetic construct
  REGION 1..17: note = Synthetic
NIYSNPNRNYYADSVEG

SEQ ID NO: 66 - protein, 120 aa, synthetic construct
  REGION 1..120: note = Synthetic
QVQLVQSGGGLVQPGGSLRLSCAASGFTFSSYAMSWVRQAPGKGLEWVSNIYSNPDRTYYADSVEGRFTISRDNSKNTLYLQMNSLRAEDTAVYYCAKAPGDYTAYFDYWGQGTLVTVSS

SEQ ID NO: 67 - protein, 120 aa, synthetic construct
  REGION 1..120: note = Synthetic
QVQLVQSGGGLVQPGGSLRLSCAASGFTFSSYAMSWVRQAPGKGLEWVSNIYSNPERTYYADSVEGRFTISRDNSKNTLYLQMNSLRAEDTAVYYCAKAPGDYTAYFDYWGQGTLVTVSS

SEQ ID NO: 68 - protein, 120 aa, synthetic construct
  REGION 1..120: note = Synthetic
QVQLVQSGGGLVQPGGSLRLSCAASGFTFSSYAMSWVRQAPGKGLEWVSNIYSNPGRTYYADSVEGRFTISRDNSKNTLYLQMNSLRAEDTAVYYCAKAPGDYTAYFDYWGQGTLVTVSS

SEQ ID NO: 69 - protein, 120 aa, synthetic construct
  REGION 1..120: note = Synthetic
QVQLVQSGGGLVQPGGSLRLSCAASGFTFSSYAMSWVRQAPGKGLEWVSNIYSNPQRTYYADSVEGRFTISRDNSKNTLYLQMNSLRAEDTAVYYCAKAPGDYTAYFDYWGQGTLVTVSS

SEQ ID NO: 70 - protein, 120 aa, synthetic construct
  REGION 1..120: note = Synthetic
QVQLVQSGGGLVQPGGSLRLSCAASGFTFSSYAMSWVRQAPGKGLEWVSNIYSNPSRTYYADSVEGRFTISRDNSKNTLYLQMNSLRAEDTAVYYCAKAPGDYTAYFDYWGQGTLVTVSS

SEQ ID NO: 71 - protein, 120 aa, synthetic construct
  REGION 1..120: note = Synthetic
QVQLVQSGGGLVQPGGSLRLSCAASGFTFSSYAMSWVRQAPGKGLEWVSNIYSNPNRAYYADSVEGRFTISRDNSKNTLYLQMNSLRAEDTAVYYCAKAPGDYTAYFDYWGQGTLVTVSS

SEQ ID NO: 72 - protein, 120 aa, synthetic construct
  REGION 1..120: note = Synthetic
QVQLVQSGGGLVQPGGSLRLSCAASGFTFSSYAMSWVRQAPGKGLEWVSNIYSNPNREYYADSVEGRFTISRDNSKNTLYLQMNSLRAEDTAVYYCAKAPGDYTAYFDYWGQGTLVTVSS

SEQ ID NO: 73 - protein, 120 aa, synthetic construct
  REGION 1..120: note = Synthetic
QVQLVQSGGGLVQPGGSLRLSCAASGFTFSSYAMSWVRQAPGKGLEWVSNIYSNPNRGYYADSVEGRFTISRDNSKNTLYLQMNSLRAEDTAVYYCAKAPGDYTAYFDYWGQGTLVTVSS

SEQ ID NO: 74 - protein, 120 aa, synthetic construct
  REGION 1..120: note = Synthetic
QVQLVQSGGGLVQPGGSLRLSCAASGFTFSSYAMSWVRQAPGKGLEWVSNIYSNPNRNYYADSVEGRFTISRDNSKNTLYLQMNSLRAEDTAVYYCAKAPGDYTAYFDYWGQGTLVTVSS

SEQ ID NO: 75 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPDRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPERTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 77 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPGRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 78 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPQRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 79 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPSRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPNRAYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 81 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPNREYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPNRGYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 466 aa, synthetic construct
  REGION 1..466: note = Synthetic
MGWSCIILFLVATATGVHSQVQLVQSGGGLVQPGGSLRLSCAASGFTFSSYAMSWVRQAPGKGLEWVSNIYSNPNRNYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - skipped

SEQ ID NO: 85 - protein, 447 aa, synthetic construct
  REGION 1..447: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 447 aa, synthetic construct
  REGION 1..447: note = Synthetic
QVQLVQSGGGLVQPGGSLRLSCAASGFTFSSYAMSWVRQAPGKGLEWVSGINGNGDYTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 447 aa, synthetic construct
  REGION 1..447: note = Synthetic
QVQLVQSGGGLVQPGGSLRLSCAASGFTFSSYAMSWVRQAPGKGLEWVSAIGGSGSGTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 88 - protein, 447 aa, synthetic construct
  REGION 1..447: note = Synthetic
QVQLVQSGGGLVQPGGSLRLSCAASGFTFSSYAMSWVRQAPGKGLEWVSNIYSNPNRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 447 aa, synthetic construct
  REGION 1..447: note = Synthetic
QVQLVQSGGGLVQPGGSLRLSCAASGFTFSSYAMSWVRQAPGKGLEWVSNINGPGNGTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 90 - protein, 447 aa, synthetic construct
  REGION 1..447: note = Synthetic
QVQLVQSGGGLVQPGGSLRLSCAASGFTFSSYAMSWVRQAPGKGLEWVSNIYSNPNRTYYADSVKGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 91 - protein, 447 aa, synthetic construct
  REGION 1..447: note = Synthetic
QVQLVQSGGGLVQPGGSLRLSCAASGFTFSSYAMSWVRQAPGKGLEWVSAISGSGGSTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 92 - protein, 447 aa, synthetic construct
  REGION 1..447: note = Synthetic
QVQLVQSGGGLVQPGGSLRLSCAASGFTFSSYAMSWVRQAPGKGLEWVSNIYSNPDRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 93 - protein, 447 aa, synthetic construct
  REGION 1..447: note = Synthetic
QVQLVQSGGGLVQPGGSLRLSCAASGFTFSSYAMSWVRQAPGKGLEWVSNIYSNPERTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 94 - protein, 447 aa, synthetic construct
  REGION 1..447: note = Synthetic
QVQLVQSGGGLVQPGGSLRLSCAASGFTFSSYAMSWVRQAPGKGLEWVSNIYSNPGRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 95 - protein, 447 aa, synthetic construct
  REGION 1..447: note = Synthetic
QVQLVQSGGGLVQPGGSLRLSCAASGFTFSSYAMSWVRQAPGKGLEWVSNIYSNPQRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 96 - protein, 447 aa, synthetic construct
  REGION 1..447: note = Synthetic
QVQLVQSGGGLVQPGGSLRLSCAASGFTFSSYAMSWVRQAPGKGLEWVSNIYSNPSRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 97 - protein, 447 aa, synthetic construct
  REGION 1..447: note = Synthetic
QVQLVQSGGGLVQPGGSLRLSCAASGFTFSSYAMSWVRQAPGKGLEWVSNIYSNPNRAYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 98 - protein, 447 aa, synthetic construct
  REGION 1..447: note = Synthetic
QVQLVQSGGGLVQPGGSLRLSCAASGFTFSSYAMSWVRQAPGKGLEWVSNIYSNPNREYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 447 aa, synthetic construct
  REGION 1..447: note = Synthetic
QVQLVQSGGGLVQPGGSLRLSCAASGFTFSSYAMSWVRQAPGKGLEWVSNIYSNPNRGYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 100 - protein, 447 aa, synthetic construct
  REGION 1..447: note = Synthetic
QVQLVQSGGGLVQPGGSLRLSCAASGFTFSSYAMSWVRQAPGKGLEWVSNIYSNPNRNYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 101 - protein, 447 aa, synthetic construct
  REGION 1..447: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYRNYNYGDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 102 - protein, 447 aa, synthetic construct
  REGION 1..447: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYDPTNYYDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 103 - protein, 447 aa, synthetic construct
  REGION 1..447: note = Synthetic
QVQLVQSGGGLVQPGGSLRLSCAASGFTFSSYAMSWVRQAPGKGLEWVSAISGSGGSTYYADSVKGRFTISRDNSKNTLYLQMNSLRAEDTAVYYCAKASYRSTNYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 104 - protein, 215 aa, synthetic construct
  REGION 1..215: note = Synthetic
ELVLTQSPATLSLSPGERATLSCRASQSVSSYLAWYQQKPGQAPRLLIYGASSRATGIPDRFSGSGSGTDFTLTISRLEPEDFAVYYCQQYGSSPPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 214 aa, synthetic construct
  REGION 1..214: note = Synthetic
ELVLTQSPATLSLSPGERATLSCRASQSVSSYLAWYQQKPGQAPRLLIYGASSRATGIPDRFSGSGSGTDFTLTISRLEPEDFAVYYCQQYGSYPPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 106 - protein, 17 aa, synthetic construct
  REGION 1..17: note = Synthetic
  VAR_SEQ 9: note = X - X is any amino acids other than S and T.
NIYSNPNRXYYADSVEG

SEQ ID NO: 107 - protein, 17 aa, synthetic construct
  REGION 1..17: note = Synthetic
  VAR_SEQ 9: note = X - X is any amino acid other than S, T and C.
NIYSNPNRXYYADSVEG

SEQ ID NO: 108 - protein, 447 aa, synthetic construct
  REGION 1..447: note = Synthetic
  VAR_SEQ 58: note = X - X is any amino acid other than S and T
QVQLVQSGGGLVQPGGSLRLSCAASGFTFSSYAMSWVRQAPGKGLEWVSNIYSNPNRXYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 109 - protein, 466 aa, synthetic construct
  REGION 1..466: note = Synthetic
  VAR_SEQ 77: note = X - X is any amino acid other than T and S
MGWSCIILFLVATATGVHSQVQLVQSGGGLVQPGGSLRLSCAASGFTFSSYAMSWVRQAPGKGLEWVSNIYSNPNRXYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 110 - protein, 447 aa, synthetic construct; identical to SEQ ID NO: 108
  REGION 1..447: note = Synthetic
  VAR_SEQ 58: note = X - X is any amino acid other than S, T and C

SEQ ID NO: 111 - protein, 466 aa, synthetic construct; identical to SEQ ID NO: 109
  REGION 1..466: note = Synthetic
  VAR_SEQ 77: note = X - X is any amino acid other than T, S and C

SEQ ID NO: 112 - protein, 17 aa, synthetic construct
  REGION 1..17: note = Synthetic
  VAR_SEQ 7: note = X - X is any amino acid other than N
NIYSNPXRTYYADSVEG

SEQ ID NO: 113 - protein, 17 aa, synthetic construct
  REGION 1..17: note = Synthetic
  VAR_SEQ 7: note = X - X is any amino acid other than N and C
NIYSNPXRTYYADSVEG

SEQ ID NO: 114 - protein, 17 aa, synthetic construct
  REGION 1..17: note = Synthetic
  VAR_SEQ 7: note = X - X is G, D, S, E, Q or N
NIYSNPXRTYYADSVEG

SEQ ID NO: 115 - protein, 447 aa, synthetic construct
  REGION 1..447: note = Synthetic
  VAR_SEQ 56: note = X - X is any amino acid other than N
QVQLVQSGGGLVQPGGSLRLSCAASGFTFSSYAMSWVRQAPGKGLEWVSNIYSNPXRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 116 - protein, 466 aa, synthetic construct
  REGION 1..466: note = Synthetic
  VAR_SEQ 75: note = X - X is any amino acid other than N
MGWSCIILFLVATATGVHSQVQLVQSGGGLVQPGGSLRLSCAASGFTFSSYAMSWVRQAPGKGLEWVSNIYSNPXRTYYADSVEGRFTISRDNSKNTLYLQMNSLRAEDTAVYYCAKAPGDYTAYFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 117 - protein, 447 aa, synthetic construct; identical to SEQ ID NO: 115
  REGION 1..447: note = Synthetic
  VAR_SEQ 56: note = X - X is any amino acid other than N and C

SEQ ID NO: 118 - protein, 466 aa, synthetic construct; identical to SEQ ID NO: 116
  REGION 1..466: note = Synthetic
  VAR_SEQ 75: note = X - X is any amino acid other than N and C

SEQ ID NO: 119 - protein, 447 aa, synthetic construct; identical to SEQ ID NO: 115
  REGION 1..447: note = Synthetic
  VAR_SEQ 56: note = X - X is is G, D, S, E, Q or N

SEQ ID NO: 120 - protein, 466 aa, synthetic construct; identical to SEQ ID NO: 116
  REGION 1..466: note = Synthetic
  VAR_SEQ 75: note = X - X is G, D, S, E, Q or N